(19) United States
(12) Patent Application Publication
(10) Pub. No.: US 2018/0057507 A1
(43) Pub. Date: Mar. 1, 2018

(54) INHIBITORS OF DUAL LEUCINE ZIPPER (DLK) KINASE FOR THE TREATMENT OF DISEASE

(71) Applicant: Board of Regents, The University of Texas System, Austin, TX (US)

(72) Inventors: Michael J. SOTH, Sugar Land, TX (US); Philip JONES, Houston, TX (US); James RAY, Houston, TX (US); Gang LIU, Sugar Land, TX (US); Kang LE, Sugar Land, TX (US); Jason CROSS, Pearland, TX (US)

(21) Appl. No.: 15/688,554

(22) Filed: Aug. 28, 2017

Related U.S. Application Data
(60) Provisional application No. 62/380,822, filed on Aug. 29, 2016.

(51) Int. Cl. C07D 498/04; C07D 405/14; A61K 31/4439; C07D 401/04; A61K 31/5377; C07D 487/04; A61K 31/4985; A61K 31/5383; A61K 31/553; C07D 401/14; A61K 31/496; C07D 491/048; C07D 413/14

(52) U.S. Cl. CPC C07D 498/04; C07D 405/14; A61K 31/4439; C07D 401/04; A61K 31/5377; C07D 487/04; C07D 413/14; A61K 31/5383; A61K 31/553; C07D 401/14; A61K 31/496; C07D 491/048; A61K 31/4985

(57) ABSTRACT

Disclosed herein are compounds which inhibit the kinase activity of dual leucine zipper (DLK) kinase (MAP3K12), pharmaceutical compositions, and methods of treatment of DLK-mediated diseases, such as neurological diseases that result from traumatic injury to central nervous system and peripheral nervous system neurons (e.g. stroke, traumatic brain injury, spinal cord injury), or that result from a chronic neurodegenerative condition (e.g. Alzheimer's disease, frontotemporal dementia, Parkinson's disease, Huntington's disease, amyotrophic lateral sclerosis, spinocerebellar ataxia, progressive supranuclear palsy, Lewy body disease, Kennedy's disease, and other related conditions), from neuropathies resulting from neurological damage (chemotherapy-induced peripheral neuropathy, diabetic neuropathy, and related conditions) and from cognitive disorders caused by pharmacological intervention (e.g. chemotherapy induced cognitive disorder, also known as chemobrain).

This application claims the benefit of U.S. Provisional Application No. 62/380,822, filed Aug. 29, 2016, the entirety of which is hereby incorporated by reference as if written herein in its entirety.

Disclosed herein are new substituted imidazole substituted aminopyridines and compositions and their application as pharmaceuticals for the treatment of disease. Methods of inhibition of the kinase activity of dual leucine zipper in a human or animal subject are also provided for the treatment of diseases such as neurological diseases that result from traumatic injury to central nervous system and peripheral nervous system neurons, neurodegenerative conditions, neuropathies resulting from neurological damage, and treatment of pain and cognitive disorders caused by pharmacological intervention.

Dual leucine zipper kinase (DLK) is a member of the mixed lineage kinase (MLK) family that is required for stress-induced neuronal activation of c-Jun N-terminal kinases (JNK). In turn, JNK is implicated in pathways important to cellular regulation including apoptosis and cell proliferation. JNK has been implicated in both naturally occurring cell death and pathological death of neurons. For this reason, compounds that inhibit DLK, and therefore modulate the activity of JNK, are attractive candidates for use both in neuroprotection and to prevent neurodegeneration.

Novel compounds and pharmaceutical compositions, certain of which have been found to inhibit the kinase activity of DLK have been discovered, together with methods of synthesizing and using the compounds including methods for the treatment of DLK-mediated diseases in a patient by administering the compounds.

### DETAILED DESCRIPTION OF THE INVENTION

In certain embodiments of the present invention, compounds have structural Formula I:

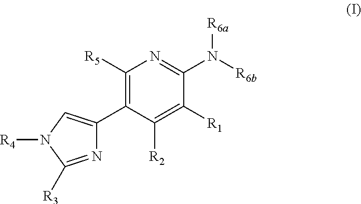

or a salt or ester thereof, wherein:
R₁ is selected from H, halo, alkyl, cycloalkyl, haloalkyl, halocycloalkyl, alkoxy, cycloalkoxy, haloalkoxy, and halocycloalkoxy;
R₂ is selected from H, halo, C₁₋₄ alkyl, and C₁₋₄ alkoxy;
R₃ and R₄ are independently selected from H, alkyl, heteroalkyl, cycloalkyl, heterocycloalkyl, and haloalkyl, any of which is optionally substituted with one to three R₇ groups; or R₃ and R₄ together, in combination with the intervening atoms, form a ring containing atoms selected from C, N, and O, said ring being optionally substituted with one to three R₇ groups;
R₅ is selected from H, halo, C₁₋₄ alkyl, and C₁₋₄ alkoxy;
R₆ₐ and R_{6b} are independently selected from H and C₁₋₄ alkyl;
R₇ is selected from acyl, alkoxy, alkyl, amino, cyano, halo, haloalkyl, haloalkoxy, hydroxyl, sulfonylalkyl, sulfonamidoalkyl, carboxyl, cycloalkyl, heterocycloalkyl, aryl, arylalkyl, and heteroaryl, any of which is optionally substituted with one to three R₈ groups; and
R₈ is selected from C₁₋₄ alkyl, C₁₋₄ alkoxy, halo, hydroxy, oxo, hydroxyalkyl, amino, carboxyl, cyano, C₃₋₆ cycloalkyl, heterocycloalkyl, C₁₋₄ haloalkyl, C₁₋₄ haloalkoxy, aryl, and heteroaryl; or two R₈, in combination with the intervening atoms, form a 4-7 membered ring consisting of atoms selected from C, N, and O, said ring being optionally substituted with one to three groups selected from amino, halo, and hydroxy.

Certain compounds disclosed herein possess useful DLK inhibiting activity, and may be used in the treatment or prophylaxis of a disease or condition in which DLK plays an active role. Thus, in broad aspect, certain embodiments also provide pharmaceutical compositions comprising one or more compounds disclosed herein together with a pharmaceutically acceptable carrier, as well as methods of making and using the compounds and compositions. Certain embodiments provide methods for inhibiting DLK. Other embodiments provide methods for treating a DLK-mediated disorder in a patient in need of such treatment, comprising administering to said patient a therapeutically effective amount of a compound or composition as disclosed herein. Also provided is the use of certain compounds disclosed herein for use in the manufacture of a medicament for the treatment of a disease or condition ameliorated by the inhibition of DLK.

In certain embodiments, R₃ and R₄ are independently selected from H, alkyl, heteroalkyl, cycloalkyl, heterocycloalkyl, and haloalkyl, any of which is optionally substituted with one to three R₇ groups.

In certain embodiments, at least one of R₃ and R₄ is selected from alkyl, cycloalkyl, and alkyl substituted with cycloalkyl.

In certain embodiments, at least one of R₃ and R₄ is bicyclo[3.1.0]hexan-6-yl, and is optionally substituted with one to three R₇ groups. In certain further embodiments, the bicyclo[3.1.0]hexan-6-yl group has exo stereochemistry.

In certain embodiments, at least one of R₃ and R₄ is 3-azabicyclo[3.1.0]hexan-6-yl, and is optionally substituted with one or more R₇ groups. In certain further embodiments, the 7-azabicyclo[3.1.0]hexan-6-yl group has exo stereochemistry.

In certain embodiments, R₁ is haloalkyl.

In certain embodiments, R₁ is trifluoromethyl.

In certain embodiments, at least one of R₂ and R₅ is H.

In certain embodiments, R₂ and R₅ are H.

In certain embodiments, at least one of R₆ₐ and R_{6b} is H.

In certain embodiments, R₆ₐ and R_{6b} are H.

In certain embodiments, compounds have structural Formula II:

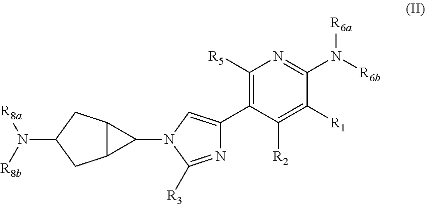

or a salt or ester thereof, wherein:
R₁ is selected from H, halo, alkyl, cycloalkyl, haloalkyl, halocycloalkyl, alkoxy, cycloalkoxy, haloalkoxy, and halocycloalkoxy;
R₂ is selected from H, halo, C₁₋₄ alkyl, and C₁₋₄ alkoxy;
R₃ is selected from H, alkyl, heteroalkyl, cycloalkyl, heterocycloalkyl, and haloalkyl, any of which is optionally substituted with one to three R₇ groups;
R₅ is selected from H, halo, C₁₋₄ alkyl, and C₁₋₄ alkoxy;
R₆ₐ and R_{6b} are independently selected from H and C₁₋₄ alkyl;
R₇ is selected from acyl, alkoxy, alkyl, amino, halo, hydroxyl, sulfonylalkyl, sulfonamidoalkyl, carboxyl, cycloalkyl, heterocycloalkyl, aryl, arylalkyl, and heteroaryl, any of which is optionally substituted with one to three R₈ groups; and
R₈ₐ and R_{8b} are independently selected from H, C₁₋₄ alkyl, C₁₋₄ alkoxy, halo, hydroxy, oxo, hydroxyalkyl, amino, carboxyl, cyano, C₃₋₆ cycloalkyl, heterocycloalkyl, C₁₋₄ haloalkyl, C₁₋₄haloalkoxy, aryl, and heteroaryl; or R₈ₐ and R_{8b}, in combination with the intervening atoms, form a 4-7 membered ring consisting of atoms selected from C, N, and O, said ring being optionally substituted with one to three groups selected from amino, halo, and hydroxy.

In certain embodiments, compounds have structural Formula III:

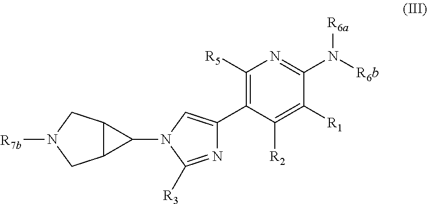

or a salt or ester thereof, wherein:
R₁ is selected from H, halo, alkyl, cycloalkyl, haloalkyl, halocycloalkyl, alkoxy, cycloalkoxy, haloalkoxy, and halocycloalkoxy;
R₂ is selected from H, halo, C₁₋₄ alkyl, and C₁₋₄ alkoxy;
R₃ is selected from H, alkyl, heteroalkyl, cycloalkyl, heterocycloalkyl, and haloalkyl, any of which is optionally substituted with one to three R₇ₐ groups;
R₅ is selected from H, halo, C₁₋₄ alkyl, and C₁₋₄ alkoxy;
R₆ₐ and R_{6b} are independently selected from H and C₁₋₄ alkyl;
R₇ₐ is selected from acyl, alkoxy, alkyl, amino, cyano, halo, haloalkyl, haloalkoxy, hydroxyl, sulfonylalkyl, sulfonamidoalkyl, carboxyl, cycloalkyl, heterocycloalkyl, aryl, arylalkyl, and heteroaryl, any of which is optionally substituted with one to three R₈ groups; and
R_{7b} is selected from H, acyl, alkyl, sulfonylalkyl, sulfonamidoalkyl, carboxyl, cycloalkyl, heterocycloalkyl, aryl, arylalkyl, and heteroaryl, any of which is optionally substituted with one to three R₈ groups; and
R₈ is selected from C₁₋₄ alkyl, C₁₋₄ alkoxy, halo, hydroxy, oxo, hydroxyalkyl, amino, carboxyl, cyano, C₃₋₆ cycloalkyl, heterocycloalkyl, C₁₋₄ haloalkyl, C₁₋₄ haloalkoxy, aryl, and heteroaryl; or two R₈, in combination with the intervening atoms, form a 4-7 membered ring consisting of atoms selected from C, N, and O, said ring being optionally substituted with one to three groups selected from amino, halo, and hydroxy.

In certain embodiments, the 7-azabicyclo[3.1.0]heptane ring has exo stereochemistry.

In certain embodiments, compounds have structural Formula IV:

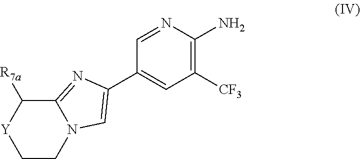

or a salt or ester thereof, wherein:
Y is selected from O, N(R_{7b}), and CH(R_{7b});
R₇ₐ is selected from H, acyl, alkoxy, alkyl, amino, cyano, halo, haloalkyl, haloalkoxy, hydroxyl, sulfonylalkyl, sulfonamidoalkyl, carboxyl, cycloalkyl, heterocycloalkyl, aryl, arylalkyl, and heteroaryl, any of which is optionally substituted with one to three R₈ groups; and
R_{7b} is selected from H, acyl, alkyl, sulfonylalkyl, sulfonamidoalkyl, carboxyl, cycloalkyl, heterocycloalkyl, aryl, arylalkyl, and heteroaryl, any of which is optionally substituted with one to three R₈ groups; and
R₈ is selected from C₁₋₄ alkyl, C₁₋₄ alkoxy, halo, hydroxy, oxo, alkoxy, hydroxyalkyl, amino, carboxyl, cyano, C₃₋₆ cycloalkyl, heterocycloalkyl, C₁₋₄ haloalkyl, C₁₋₄ haloalkoxy, aryl, and heteroaryl; or two R₈, in combination with the intervening atoms, form a 4-7 membered ring consisting of atoms selected from C, N, and O, said ring being optionally substituted with one to three groups selected from amino, halo, and hydroxy.

In certain embodiments, Y is O.

In certain embodiments, Y is N(R_{7b}).

In certain embodiments, Y is CH(R_{7b}).

In certain embodiments, compounds have structural Formula V:

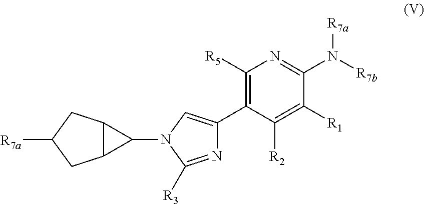

or a salt or ester thereof, wherein:
R₁ is selected from H, halo, alkyl, cycloalkyl, haloalkyl, halocycloalkyl, alkoxy, cycloalkoxy, haloalkoxy, and halocycloalkoxy;
R₂ is selected from H, halo, C₁₋₄ alkyl, and C₁₋₄ alkoxy;
R₃ is selected from H, alkyl, heteroalkyl, cycloalkyl, heterocycloalkyl, and haloalkyl, any of which is optionally substituted with one to three R_{7b} groups;
R₅ is selected from H, halo, C₁₋₄ alkyl, and C₁₋₄ alkoxy;
R₆ₐ and R_{6b} are independently selected from H and C₁₋₄ alkyl;
R₇ₐ and R_{7b} are independently selected from acyl, alkoxy, alkyl, amino, halo, hydroxyl, sulfonylalkyl, sulfonamidoalkyl, carboxyl, cycloalkyl, heterocycloalkyl, aryl, arylalkyl, and heteroaryl, any of which is optionally substituted with one to three R₈ groups; and
R₈ is selected from C₁₋₄ alkyl, C₁₋₄ alkoxy, halo, hydroxy, oxo, hydroxyalkyl, amino, carboxyl, cyano, C₃₋₆ cycloalkyl, heterocycloalkyl, C₁₋₄ haloalkyl, C₁₋₄ haloalkoxy, aryl, and heteroaryl; or two R₈, in combination with the intervening atoms, form a 4-7 membered ring consisting of atoms selected from C, N, and O, said ring being optionally substituted with one to three groups selected from amino, halo, and hydroxy.

In certain embodiments, the bicyclo[3.1.0]heptane ring has exo stereochemistry.

In certain embodiments, R₇ₐ is selected from alkyl, cycloalkyl, and heterocycloalkyl, and is optionally substituted with one to three R₈ groups.

In certain embodiments, R₇ₐ is heterocycloalkyl, and is optionally substituted with one to three R₈ groups.

In certain embodiments, R₇ₐ is selected from piperazin-1-yl, morpholin-1-yl, 1,4-diazepan-1-yl, and 1,4-oxazepan-4-yl, and is optionally substituted with one or two R₈ groups.

In certain embodiments, R₇ₐ is selected from

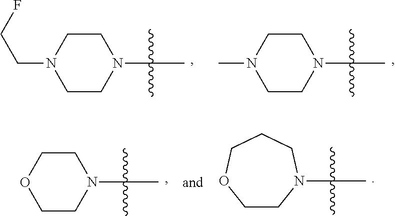

In certain embodiments, R₈ is selected from C₁₋₄ alkyl, C₁₋₄ alkoxy, halo, hydroxy, oxo, hydroxyalkyl, amino, carboxyl, cyano, C₃₋₆ cycloalkyl, heterocycloalkyl, C₁₋₄ haloalkyl, C₁₋₄haloalkoxy, aryl, and heteroaryl.

In certain embodiments, R₈ is selected from C₁₋₄ alkyl, C₁₋₄ alkoxy, halo, hydroxy, oxo, hydroxyalkyl, C₃₋₆ cycloalkyl, heterocycloalkyl, C₁₋₄ haloalkyl, and C₁₋₄ haloalkoxy.

In certain embodiments, R₈ is selected from C₁₋₄ alkyl, hydroxyalkyl, and C₁₋₄haloalkyl.

In certain embodiments, R₈ is selected from C₁₋₄ alkyl and C₁₋₄ haloalkyl.

In certain embodiments, R₈ is C₁₋₄ fluoroalkyl.

In certain embodiments, R₈ is 2-fluoroethyl.

In certain embodiments, R₈ is C₁₋₄ alkyl.

In certain embodiments, R₈ is methyl.

In certain embodiments, the compound has the structural formula chosen from:

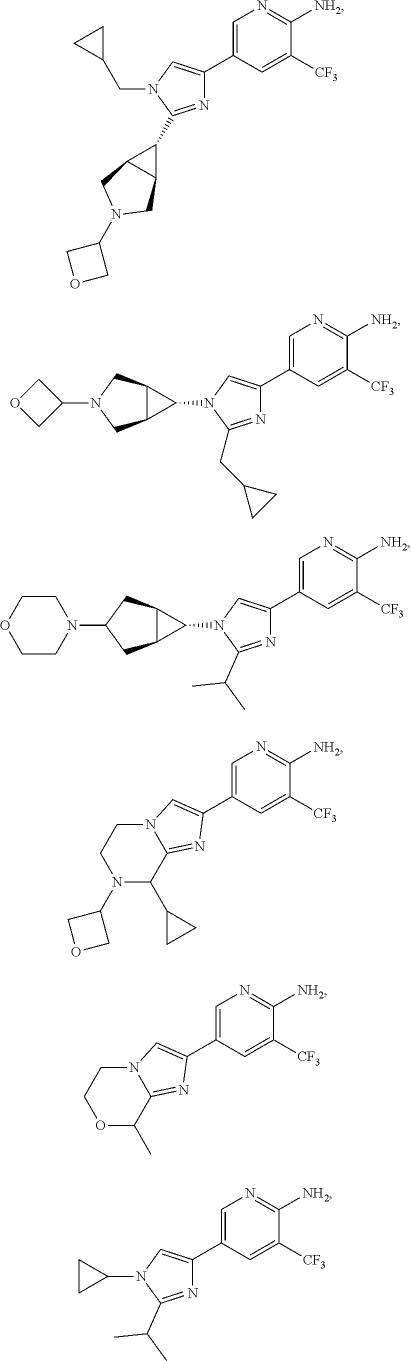

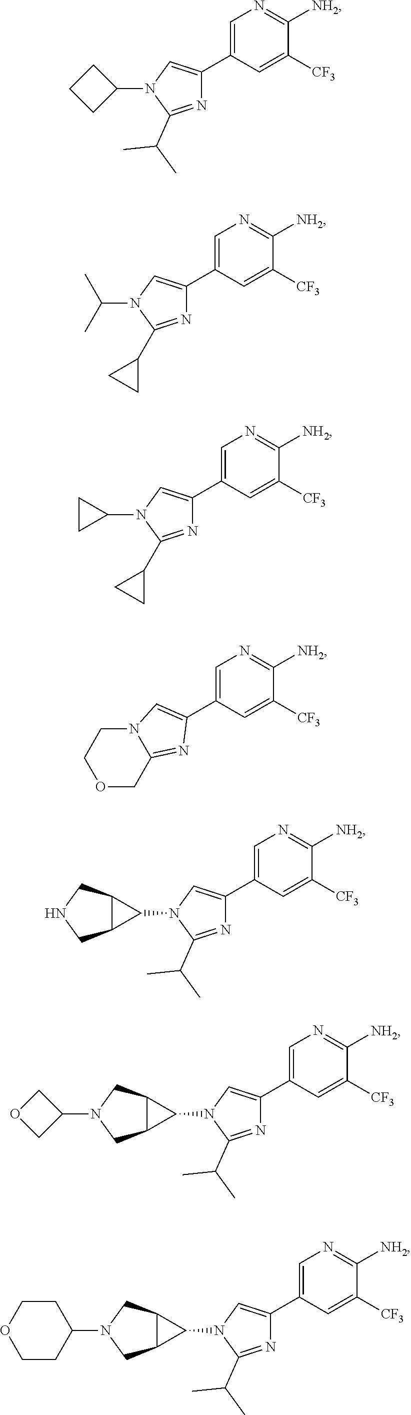

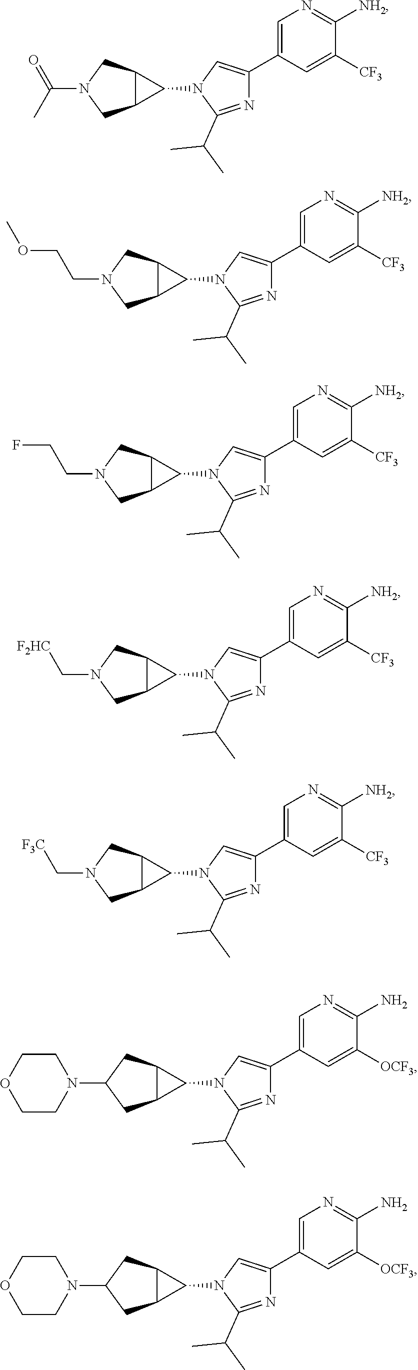

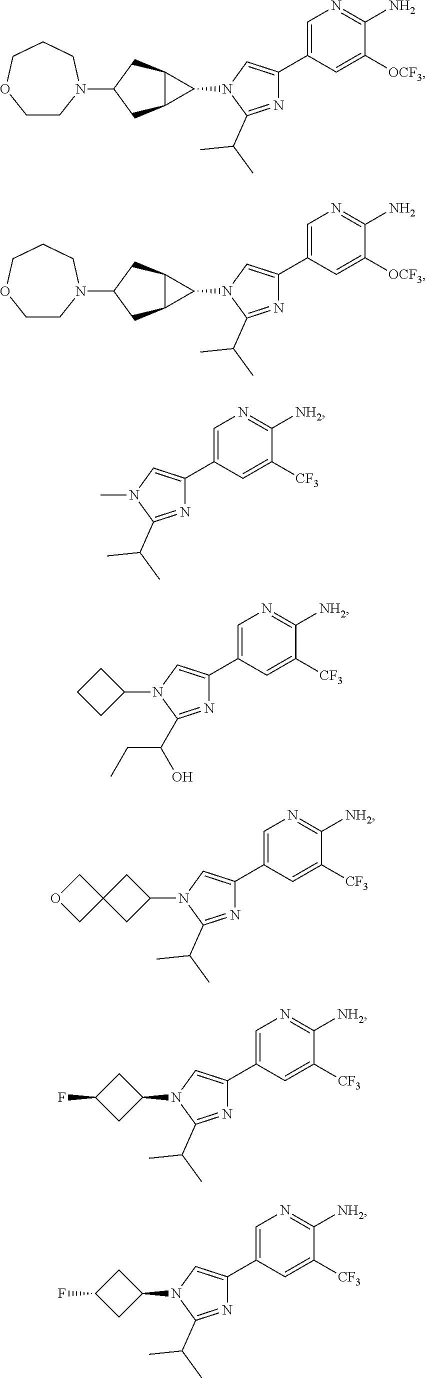

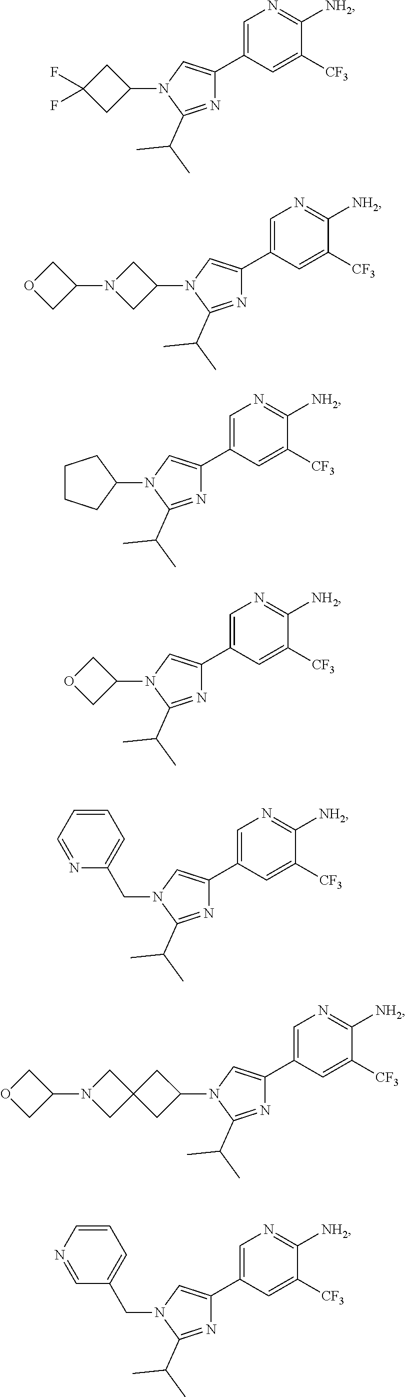

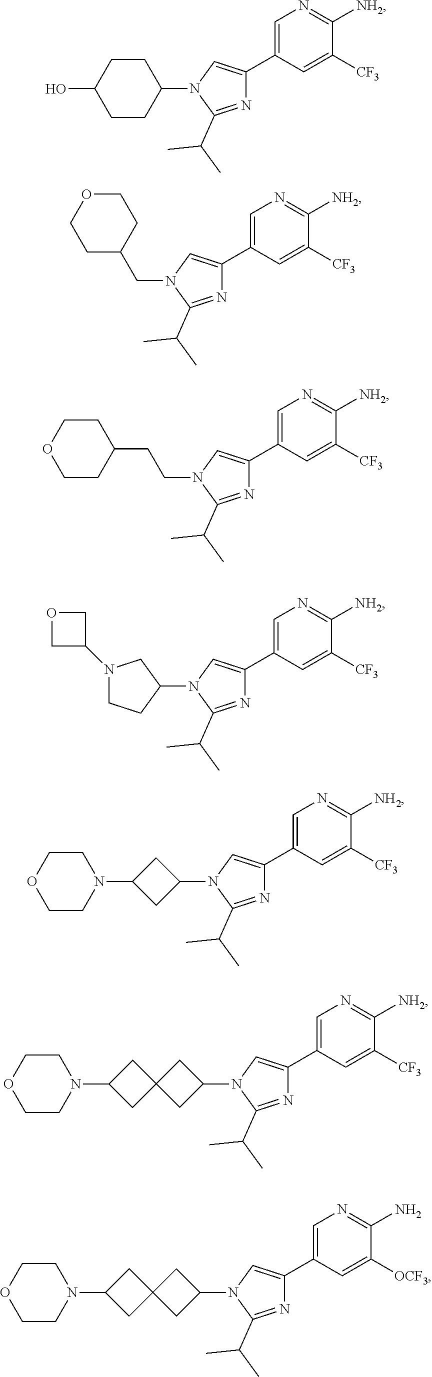

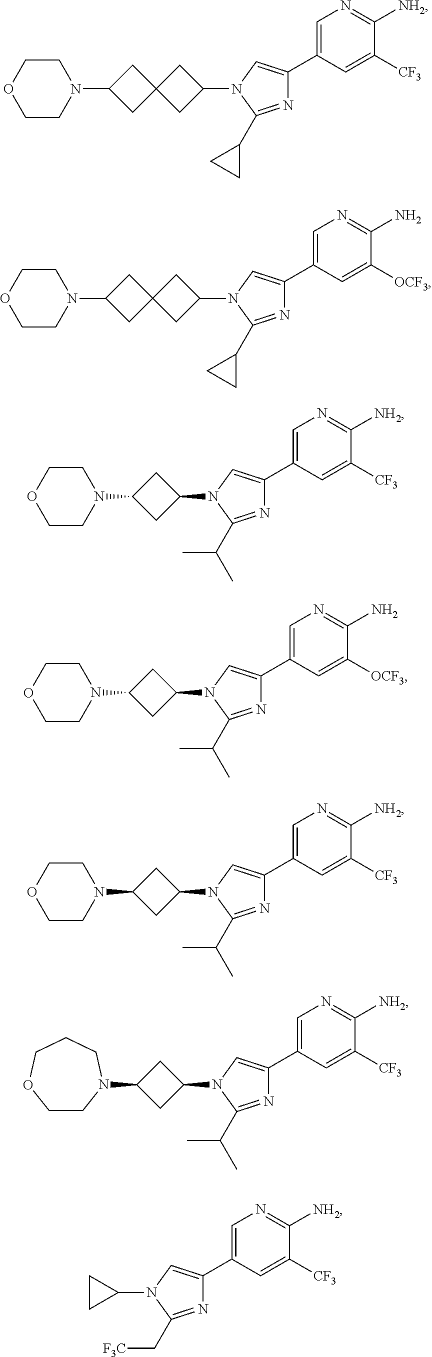

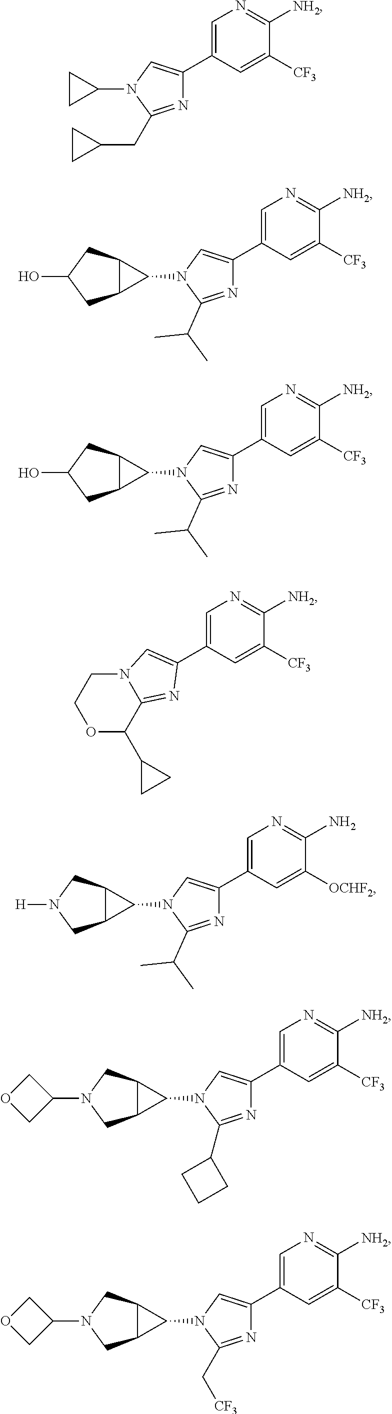

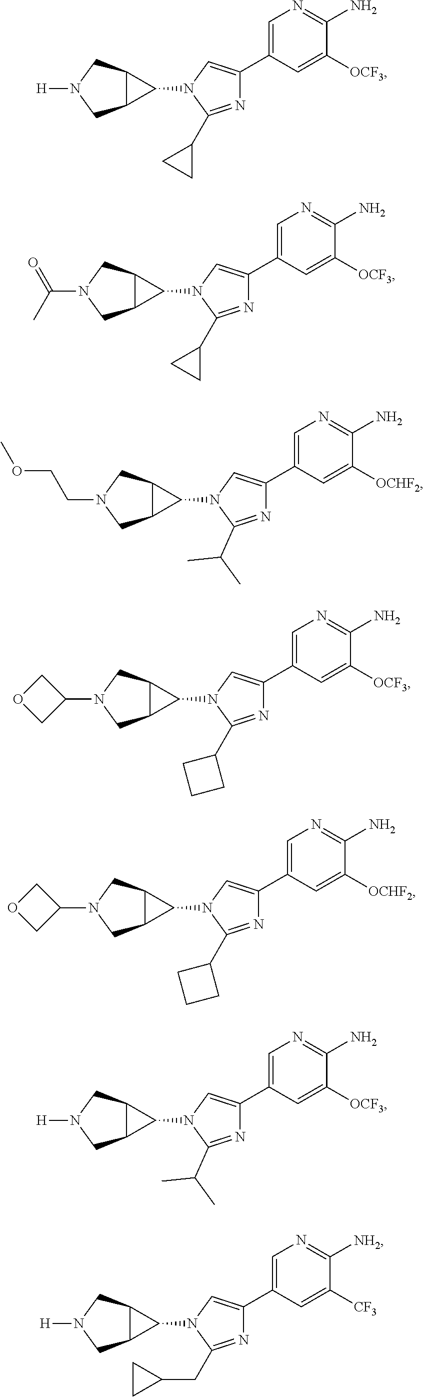

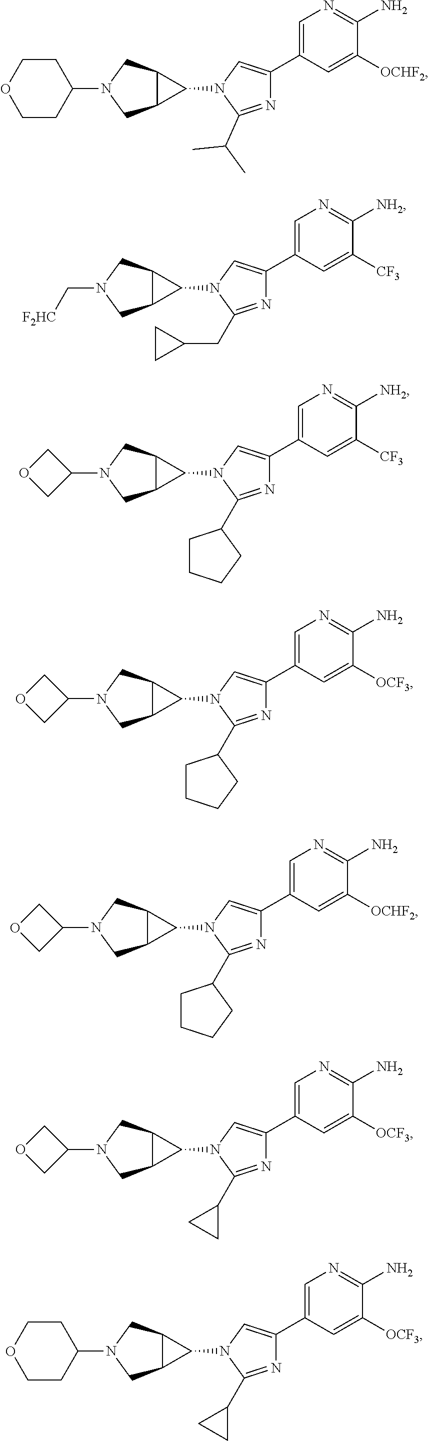

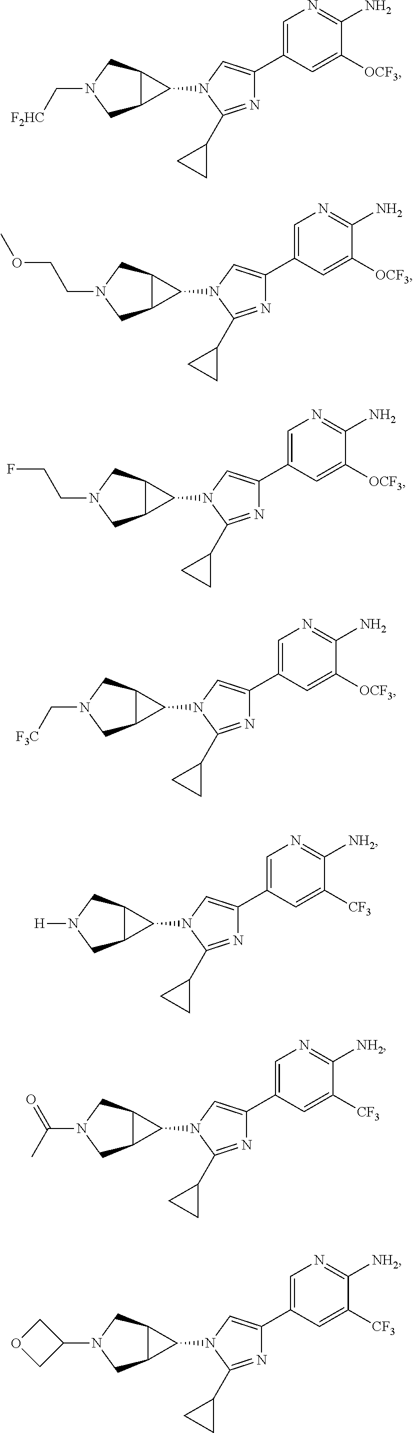

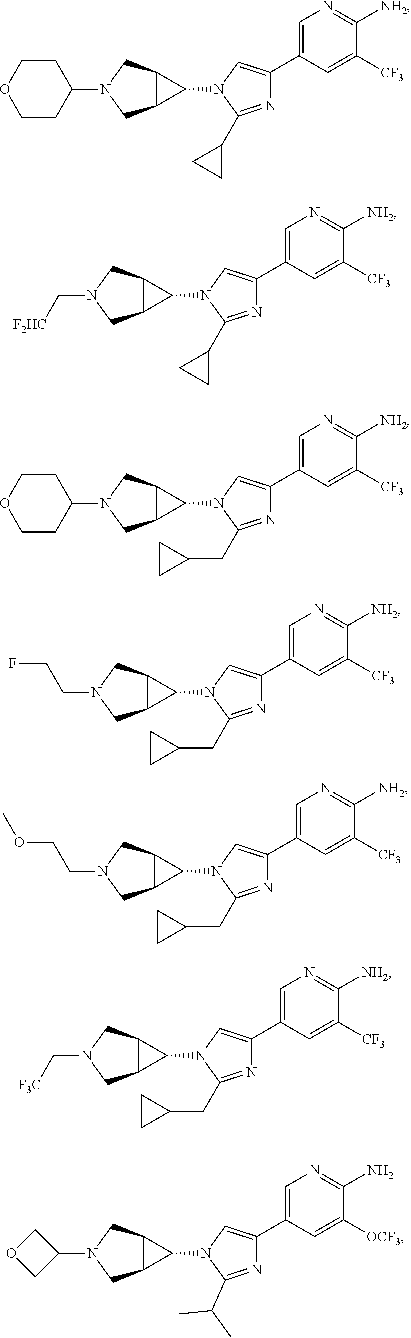

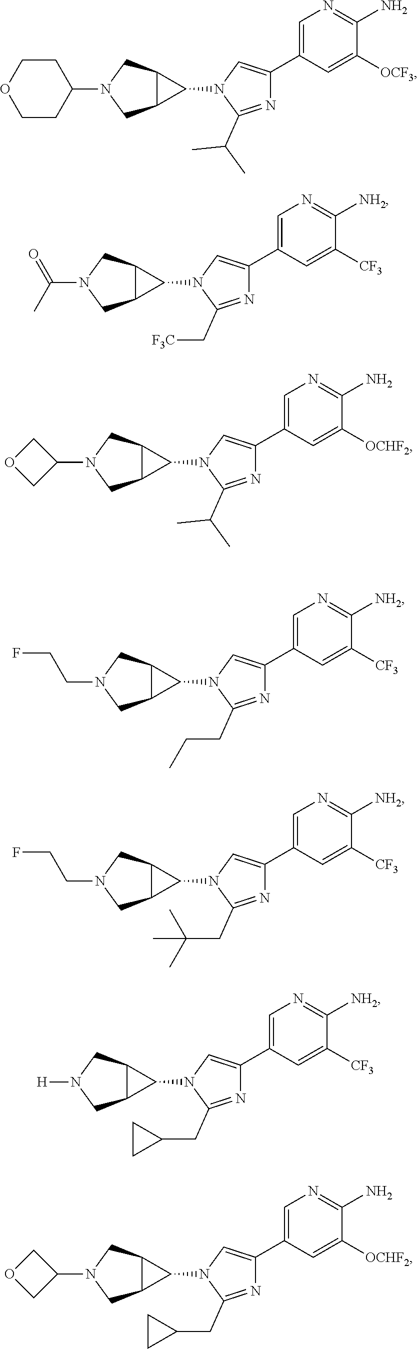

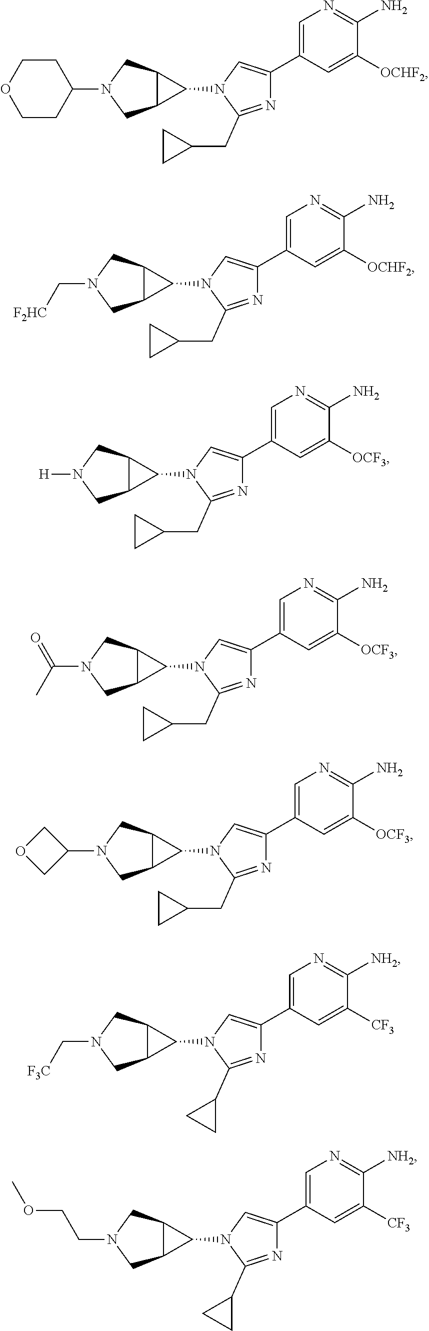

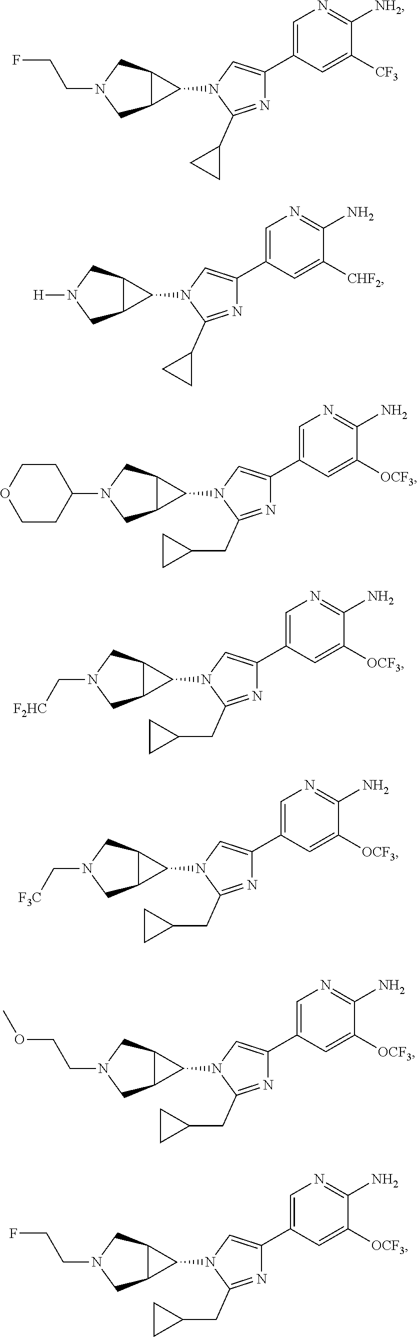

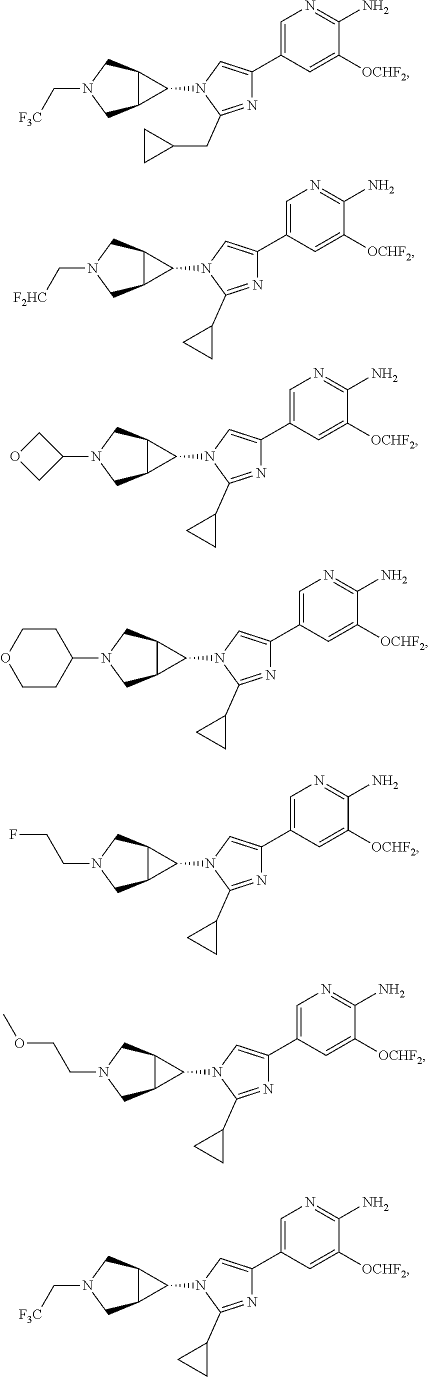

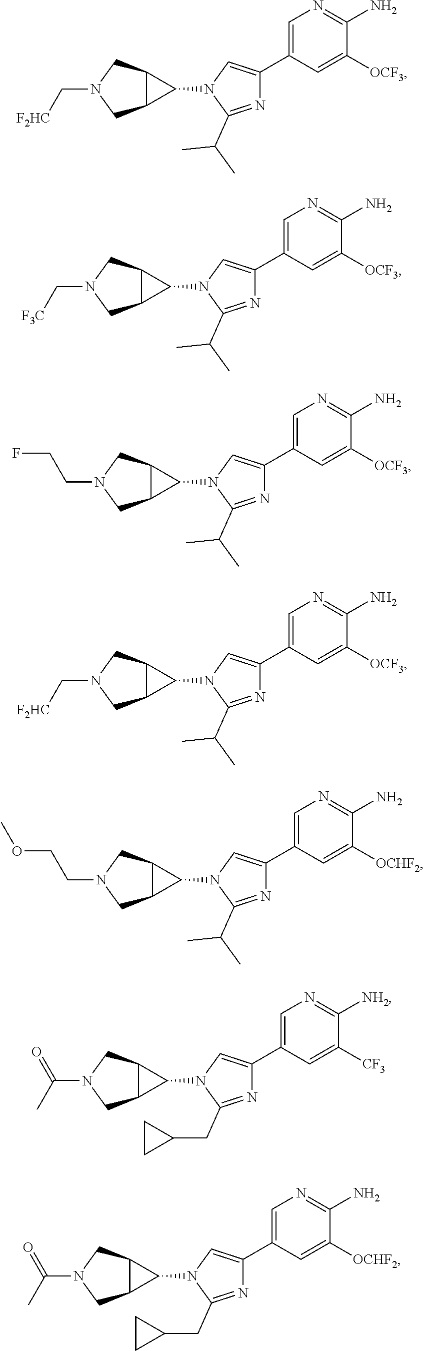

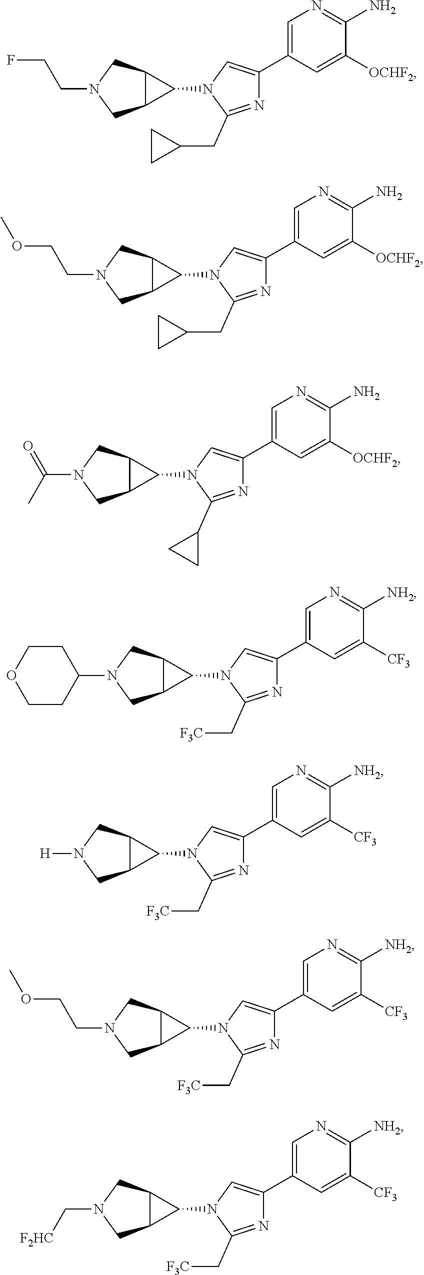

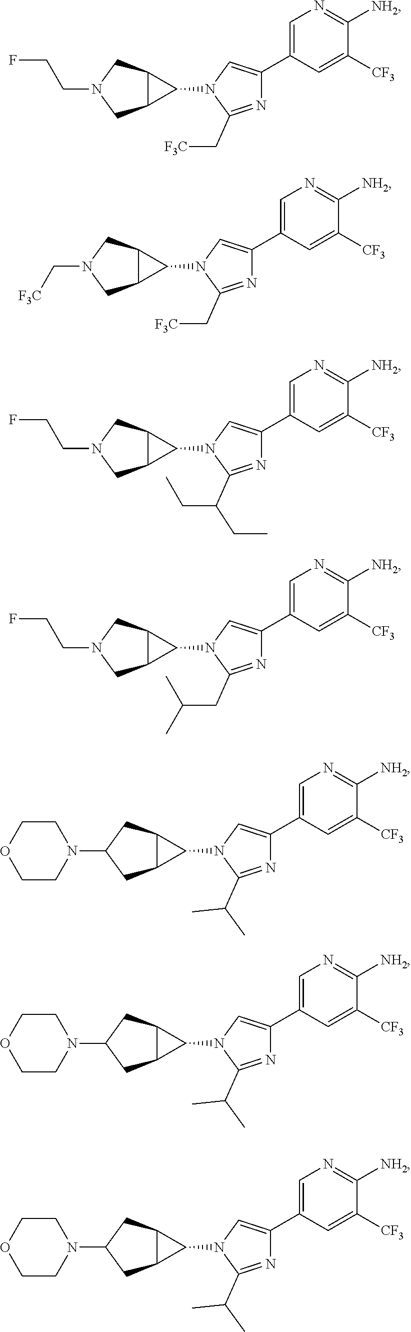

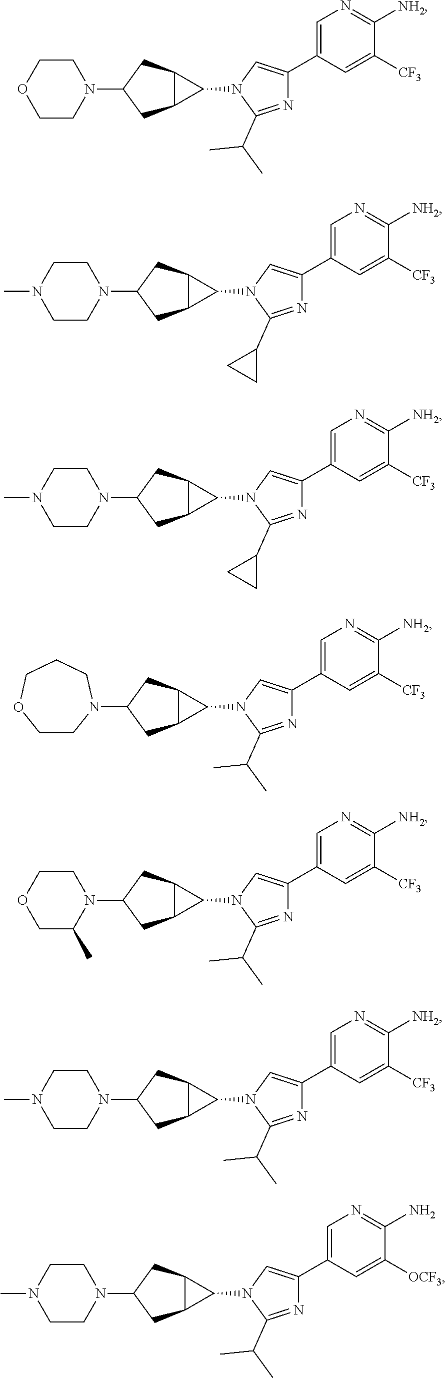

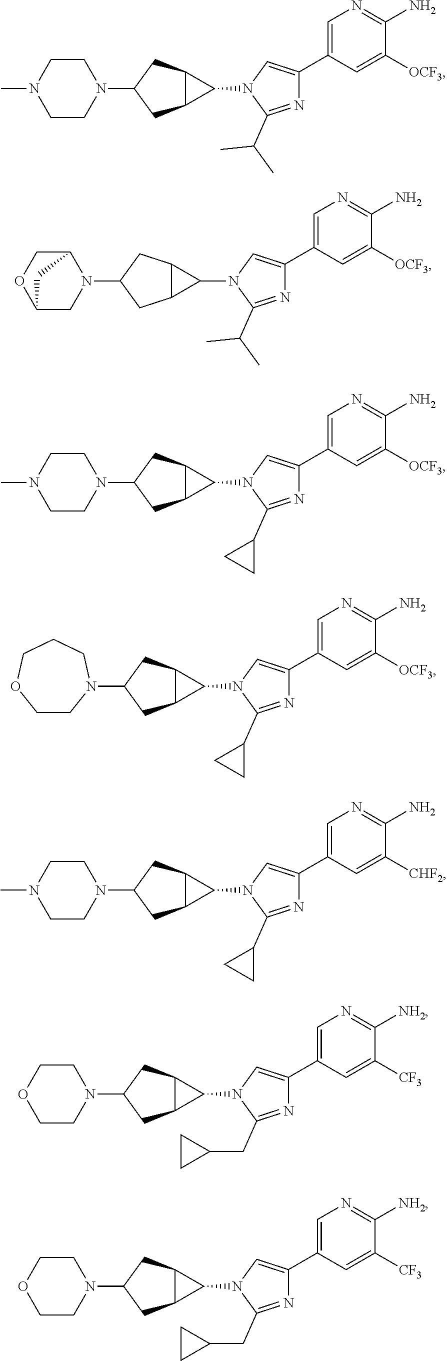

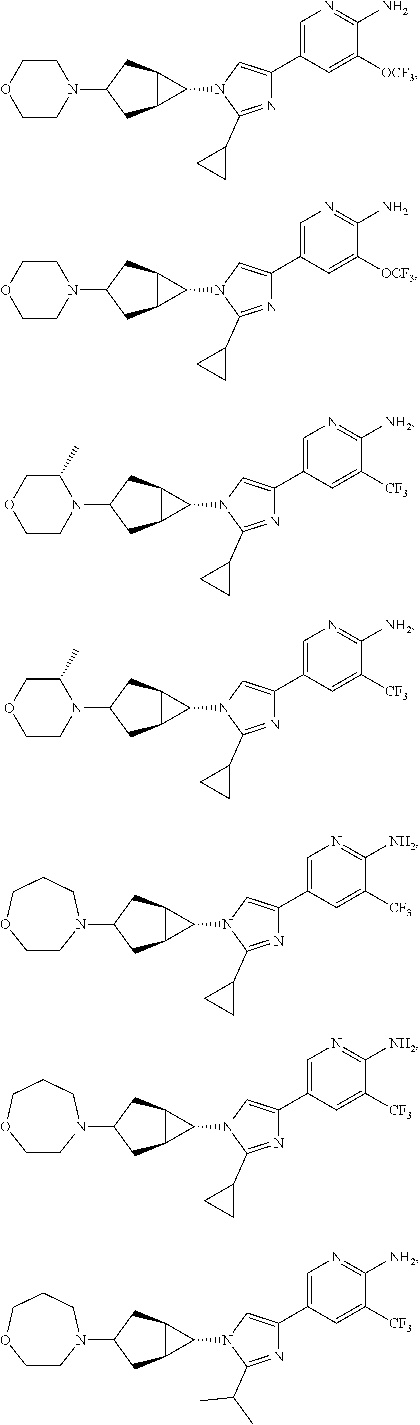

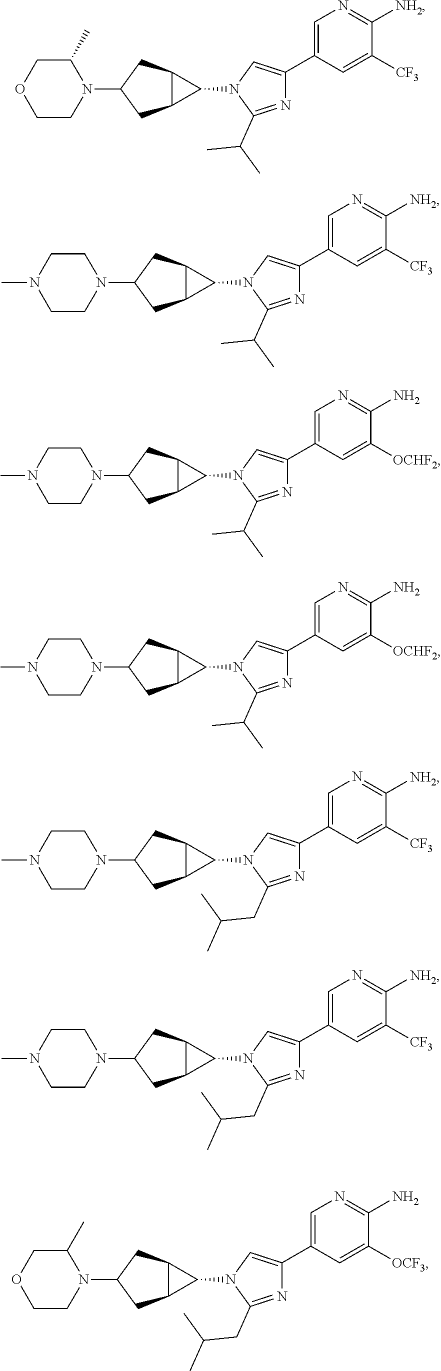

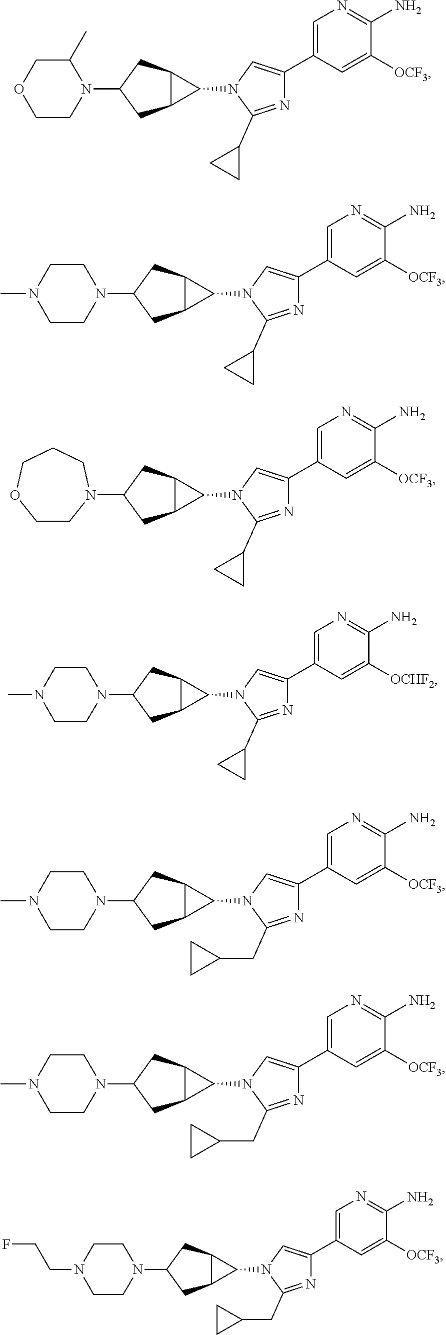

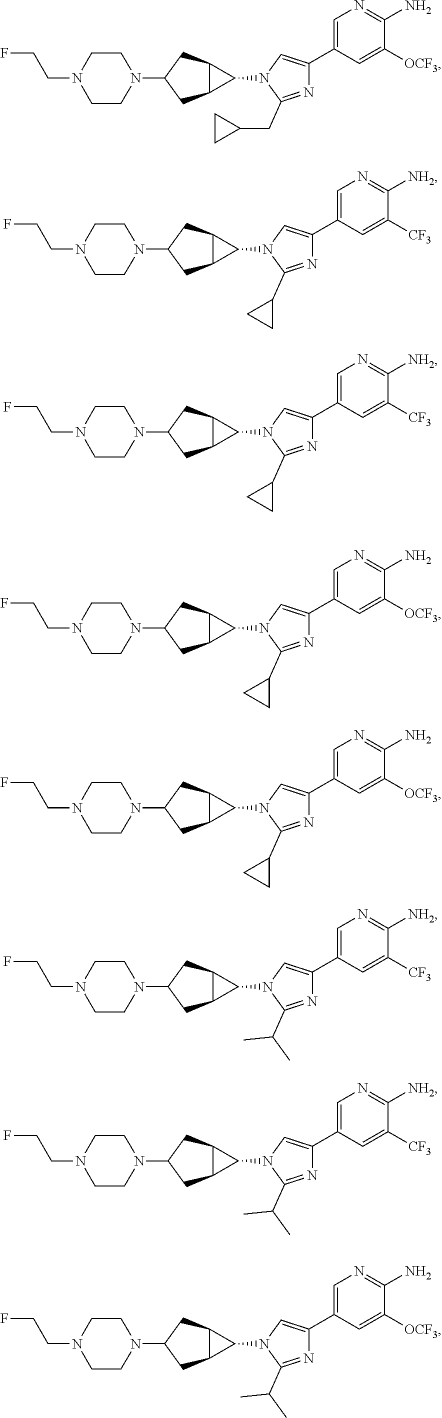

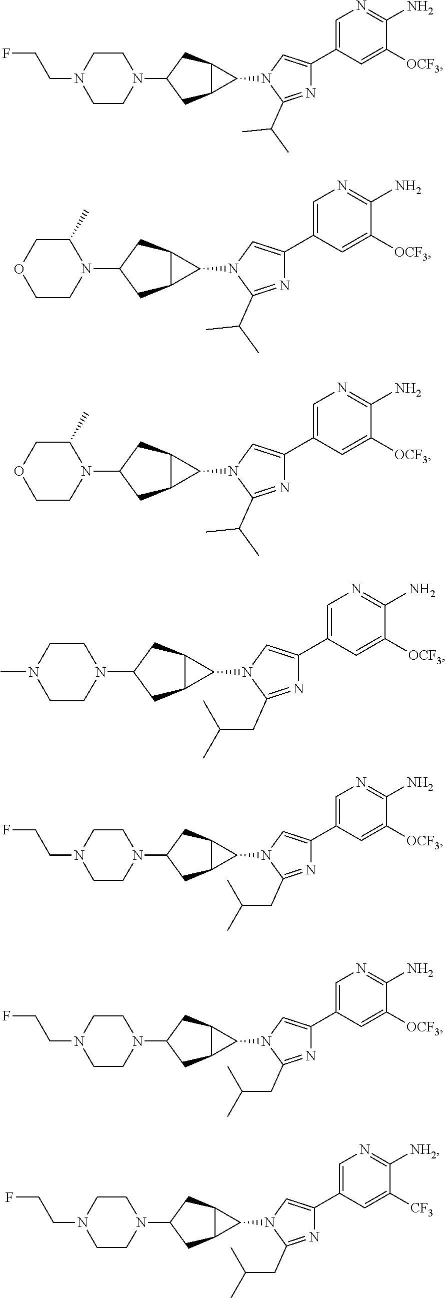

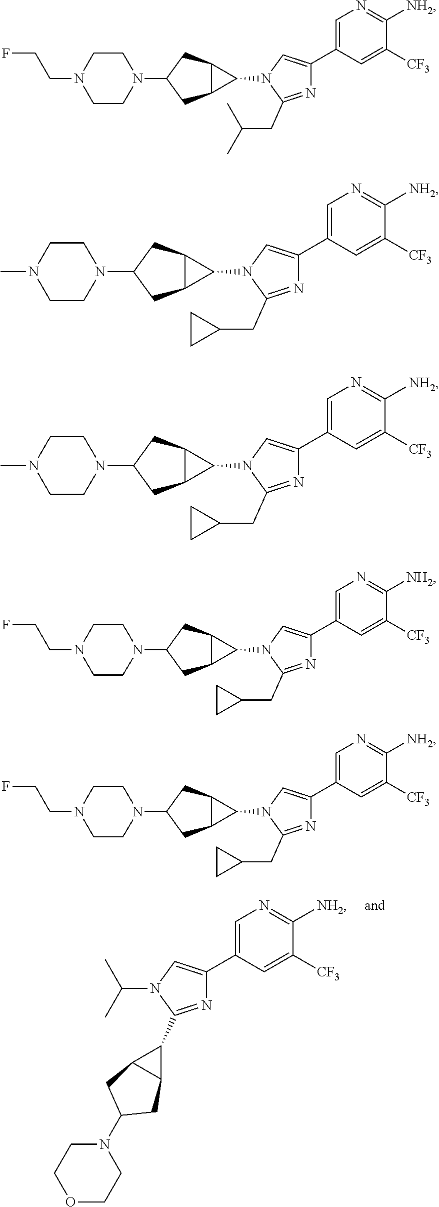

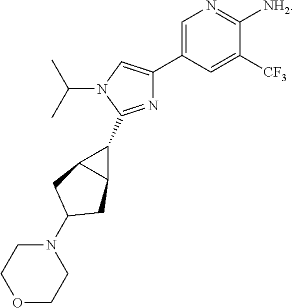

Also provided are embodiments wherein any embodiment above may be combined with any one or more of these embodiments, provided the combination is not mutually exclusive.

As used herein, two embodiments are “mutually exclusive” when one is defined to be something which is different than the other. For example, an embodiment wherein two groups combine to form a cycloalkyl is mutually exclusive with an embodiment in which one group is ethyl the other group is hydrogen. Similarly, an embodiment wherein one group is CH₂ is mutually exclusive with an embodiment wherein the same group is NH.

Also provided is a compound chosen from the Examples disclosed herein.

Also provided are methods of inhibiting at least one DLK function comprising the step of contacting DLK with a compound as described herein. The cell phenotype, cell proliferation, activity of DLK, change in biochemical output produced by active DLK, expression of DLK, or binding of DLK with a natural binding partner may be monitored. Such methods may be modes of treatment of disease, biological assays, cellular assays, biochemical assays, or the like.

Also provided herein are methods of treatment of a DLK-mediated disease comprising the administration of a therapeutically effective amount of a compound as disclosed herein, or a salt thereof, to a patient in need thereof.

In certain embodiments, the disease is chosen from a neurodegenerative disease.

Also provided herein is a compound as disclosed herein for use as a medicament.

Also provided herein is a compound as disclosed herein for use as a medicament for the treatment of a DLK-mediated disease.

Also provided is the use of a compound as disclosed herein as a medicament.

Also provided is the use of a compound as disclosed herein as a medicament for the treatment of a DLK-mediated disease.

Also provided is a compound as disclosed herein for use in the manufacture of a medicament for the treatment of a DLK-mediated disease.

Also provided is the use of a compound as disclosed herein for the treatment of a DLK-mediated disease.

Also provided herein is a method of inhibition of DLK comprising contacting DLK with a compound as disclosed herein, or a salt thereof.

Also provided herein is a method for achieving an effect in a patient comprising the administration of a therapeutically effective amount of a compound as disclosed herein, or a salt thereof, to a patient, wherein the effect is chosen from cognition enhancement.

In certain embodiments, the DLK-mediated disease is chosen from a disease that results from traumatic injury to central nervous system and peripheral nervous system neurons (e.g. stroke, traumatic brain injury, spinal cord injury), a disease that results from a chronic neurodegenerative condition (e.g. Alzheimer's disease, frontotemporal dementia, Parkinson's disease, Huntington's disease, amyotrophic lateral sclerosis, spinocerebellar ataxia, progressive supranuclear palsy, Lewy body disease, Kennedy's disease, and other related conditions), a disease that results from neuropathies resulting from neurological damage (chemotherapy-induced peripheral neuropathy, diabetic neuropathy, and related conditions) and a disease that results from cognitive disorders caused by pharmacological intervention (e.g. chemotherapy induced cognitive disorder, also known as chemobrain).

Also provided is a method of modulation of a DLK-mediated function in a subject comprising the administration of a therapeutically effective amount of a compound as disclosed herein.

Also provided is a pharmaceutical composition comprising a compound as disclosed herein, together with a pharmaceutically acceptable carrier.

In certain embodiments, the pharmaceutical composition is formulated for oral administration.

In certain embodiments, the oral pharmaceutical composition is chosen from a tablet and a capsule.

### Definitions

As used herein, the terms below have the meanings indicated.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

In certain embodiments, Markush groups, such as R₇ for example, can include subsets, such as R₇ₐ and R_{7b}, often provided for clarity.

The term “acyl,” as used herein, alone or in combination, refers to a carbonyl attached to an alkenyl, alkyl, aryl, cycloalkyl, heteroaryl, heterocycle, or any other moiety were the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon radical having one or more double bonds and containing from 2 to 20 carbon atoms. In certain embodiments, said alkenyl will comprise from 2 to 6 carbon atoms. The term “alkenylene” refers to a carbon-carbon double bond system attached at two or more positions such as ethenylene [(—CH═CH—),(—C::C—)]. Examples of suitable alkenyl radicals include ethenyl, propenyl, 2-methylpropenyl, 1,4-butadienyl and the like. Unless otherwise specified, the term “alkenyl” may include “alkenylene” groups.

The term “alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl radical containing from 1 to 20 carbon atoms. In certain embodiments, said alkyl will comprise from 1 to 10 carbon atoms. In further embodiments, said alkyl will comprise from 1 to 8 carbon atoms. Alkyl groups is optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, nonyl and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like.

The term “alkylidene,” as used herein, alone or in combination, refers to an alkenyl group in which one carbon atom of the carbon-carbon double bond belongs to the moiety to which the alkenyl group is attached.

The term “alkylthio,” as used herein, alone or in combination, refers to an alkyl thioether (R—S—) radical wherein the term alkyl is as defined above and wherein the sulfur may be singly or doubly oxidized. Examples of suitable alkyl thioether radicals include methylthio, ethylthio, n-propylthio, isopropylthio, n-butylthio, iso-butylthio, sec-butylthio, tert-butylthio, methanesulfonyl, ethanesulfinyl, and the like.

The term “alkynyl,” as used herein, alone or in combination, refers to a straight-chain or branched chain hydrocarbon radical having one or more triple bonds and containing from 2 to 20 carbon atoms. In certain embodiments, said alkynyl comprises from 2 to 6 carbon atoms. In further embodiments, said alkynyl comprises from 2 to 4 carbon atoms. The term “alkynylene” refers to a carbon-carbon triple bond attached at two positions such as ethynylene (—C:::C—, —C≡C—). Examples of alkynyl radicals include ethynyl, propynyl, hydroxypropynyl, butyn-1-yl, butyn-2-yl, pentyn-1-yl, 3-methylbutyn-1-yl, hexyn-2-yl, and the like. Unless otherwise specified, the term “alkynyl” may include “alkynylene” groups.

The terms “amido” and “carbamoyl,” as used herein, alone or in combination, refer to an amino group as described below attached to the parent molecular moiety through a carbonyl group, or vice versa. The term “C-amido” as used herein, alone or in combination, refers to a —C(O)N(RR′) group with R and R′ as defined herein or as defined by the specifically enumerated “R” groups designated. The term “N-amido” as used herein, alone or in combination, refers to a RC(O)N(R′)— group, with R and R′ as defined herein or as defined by the specifically enumerated “R” groups designated. The term “acylamino” as used herein, alone or in combination, embraces an acyl group attached to the parent moiety through an amino group. An example of an “acylamino” group is acetylamino (CH₃C(O)NH—).

The term “amino,” as used herein, alone or in combination, refers to —NRR′, wherein R and R′ are independently chosen from hydrogen, alkyl, acyl, heteroalkyl, aryl, cycloalkyl, heteroaryl, and heterocycloalkyl, any of which may themselves be optionally substituted. Additionally, R and R′ may combine to form heterocycloalkyl, either of which is optionally substituted.

The term “aryl,” as used herein, alone or in combination, means a carbocyclic aromatic system containing one, two or three rings wherein such polycyclic ring systems are fused together. The term “aryl” embraces aromatic groups such as phenyl, naphthyl, anthracenyl, and phenanthryl.

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term “arylalkynyl” or “aralkynyl,” as used herein, alone or in combination, refers to an aryl group attached to the parent molecular moiety through an alkynyl group.

The term “arylalkanoyl” or “aralkanoyl” or “aroyl,” as used herein, alone or in combination, refers to an acyl radical derived from an aryl-substituted alkanecarboxylic acid such as benzoyl, naphthoyl, phenylacetyl, 3-phenylpropionyl (hydrocinnamoyl), 4-phenylbutyryl, (2-naphthyl)acetyl, 4-chlorohydrocinnamoyl, and the like.

The term aryloxy as used herein, alone or in combination, refers to an aryl group attached to the parent molecular moiety through an oxy.

The terms “benzo” and “benz,” as used herein, alone or in combination, refer to the divalent radical C₆H₄═ derived from benzene. Examples include benzothiophene and benzimidazole.

The term “carbamate,” as used herein, alone or in combination, refers to an ester of carbamic acid (—NHCOO—) which may be attached to the parent molecular moiety from either the nitrogen or acid end, and which is optionally substituted as defined herein.

The term “O-carbamyl” as used herein, alone or in combination, refers to a —OC(O)NRR′, group-with R and R′ as defined herein.

The term “N-carbamyl” as used herein, alone or in combination, refers to a ROC(O)NR′— group, with R and R′ as defined herein.

The term “carbonyl,” as used herein, when alone includes formyl [—C(O)H] and in combination is a —C(O)— group.

The term “carboxyl” or “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl group wherein each cyclic moiety contains from 3 to 12 carbon atom ring members and which may optionally be a benzo fused ring system which is optionally substituted as defined herein. In certain embodiments, said cycloalkyl will comprise from 5 to 7 carbon atoms. In certain embodiments, said cycloalkyl will comprise a spirocycle ring system. Examples of such cycloalkyl groups include cyclopropyl, cyclobutyl, cyclopentyl, cyclohexyl, cycloheptyl, tetrahydronaphthyl, indanyl, octahydronaphthyl, 2,3-dihydro-1H-indenyl, adamantyl and the like.

The term “bicyclic ring system” as used herein refers to a group which contains two distinct rings of atoms. In certain embodiments, bicyclic ring systems contain a single atom common to both ring systems. In certain embodiments, bicyclic ring systems contain two or more atoms common to both ring systems. Examples of compounds with bicyclic ring systems include decalin, norbornane, and pinene. Further examples of compounds with bicyclic ring systems are bicyclo[1.1.1]pentane, bicyclo[3.1.0.]hexane, 1,4-diazabicyclo[2.2.2]octane, 1,5-diazabicyclo(4.3.0)non-5-ene, and 7-oxabicyclo[2.2.1]heptadiene.

The term “tricyclic ring system” as used herein refers to a group which contains three distinct rings of atoms. In certain embodiments, bicyclic ring systems contain a single atom common to two rings. In certain embodiments, bicyclic ring systems contain two or more atoms common to two rings. Examples of compounds with tricyclic ring systems include perhydroanthracene, cedrene, and taxadiene. Further examples of compounds with tricyclic ring systems are tricyclo[3.1.0.0^{2,4}]hexane, tricyclo[3.3.1.1^{3,7}]decane, and cyclopentadiene diepoxide.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or combinations thereof, fully saturated or containing from 1 to 3 degrees of unsaturation, consisting of the stated number of carbon atoms and from one to three heteroatoms chosen from N, O, and S, and wherein the N and S atoms may optionally be oxidized and the N heteroatom may optionally be quaternized. The heteroatom(s) may be placed at any interior position of the heteroalkyl group. Up to two heteroatoms may be consecutive, such as, for example, —CH₂—NH—OCH₃.

The term “heteroaryl,” as used herein, alone or in combination, refers to a 3 to 15 membered unsaturated heteromonocyclic ring, or a fused monocyclic, bicyclic, or tricyclic ring system in which at least one of the fused rings is aromatic, which contains at least one atom chosen from N, O, and S. In certain embodiments, said heteroaryl will comprise from 1 to 4 heteroatoms as ring members. In further embodiments, said heteroaryl will comprise from 1 to 2 heteroatoms as ring members. In certain embodiments, said heteroaryl will comprise from 5 to 7 atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

The terms “heterocycloalkyl” and, interchangeably, “heterocycle,” as used herein, alone or in combination, each refer to a saturated, partially unsaturated, or fully unsaturated (but nonaromatic) monocyclic, bicyclic, or tricyclic heterocyclic group containing at least one heteroatom as a ring member, wherein each said heteroatom may be independently chosen from nitrogen, oxygen, and sulfur. In certain embodiments, said heterocycloalkyl will comprise a spirocycle ring system. In certain embodiments, said hetercycloalkyl will comprise from 1 to 4 heteroatoms as ring members. In further embodiments, said hetercycloalkyl will comprise from 1 to 2 heteroatoms as ring members. In certain embodiments, said hetercycloalkyl will comprise from 3 to 8 ring members in each ring. In further embodiments, said hetercycloalkyl will comprise from 3 to 7 ring members in each ring. In yet further embodiments, said hetercycloalkyl will comprise from 5 to 6 ring members in each ring. In further embodiments, said heterocycle will comprise a bicyclic ring system. In further embodiments, said heterocycle will comprise a tricyclic ring system. In further embodiments, said heterocycle will comprise a bicyclic ring system, said bicyclic ring system comprising a ring of three atoms. In further embodiments, said heterocycle will comprise a bicyclic ring system, said bicyclic ring system comprising a ring of four atoms. In further embodiments, said heterocycle will comprise a bicyclic ring system, said bicyclic ring system comprising a ring of five atoms. In further embodiments, said heterocycle will comprise a bicyclic ring system, said bicyclic ring system comprising a pyrrolidine ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include 3-azabicyclo[3.1.0]hexan-6-yl, aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihydropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups is optionally substituted unless specifically prohibited.

The term “hydrazinyl” as used herein, alone or in combination, refers to two amino groups joined by a single bond, i.e., —N—N—.

The term “hydroxy,” as used herein, alone or in combination, refers to —OH.

The term “hydroxyalkyl,” as used herein, alone or in combination, refers to a hydroxy group attached to the parent molecular moiety through an alkyl group.

The term “imino,” as used herein, alone or in combination, refers to ═N—.

The term “iminohydroxy,” as used herein, alone or in combination, refers to ═N(OH) and ═N—O—.

The phrase “in the main chain” refers to the longest contiguous or adjacent chain of carbon atoms starting at the point of attachment of a group to the compounds of any one of the formulas disclosed herein.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “lower,” as used herein, alone or in a combination, where not otherwise specifically defined, means containing from 1 to and including 6 carbon atoms (i.e., C₁-C₆ alkyl).

The term “lower aryl,” as used herein, alone or in combination, means phenyl or naphthyl, either of which is optionally substituted as provided.

The term “lower heteroaryl,” as used herein, alone or in combination, means either 1) monocyclic heteroaryl comprising five or six ring members, of which between one and four said members may be heteroatoms chosen from N, O, and S, or 2) bicyclic heteroaryl, wherein each of the fused rings comprises five or six ring members, comprising between them one to four heteroatoms chosen from N, O, and S.

The term “lower cycloalkyl,” as used herein, alone or in combination, means a monocyclic cycloalkyl having between three and six ring members (i.e., C₃-C₆ cycloalkyl). Lower cycloalkyls may be unsaturated. Examples of lower cycloalkyl include cyclopropyl, cyclobutyl, cyclopentyl, and cyclohexyl.

The term “lower heterocycloalkyl,” as used herein, alone or in combination, means a monocyclic heterocycloalkyl having between three and six ring members, of which between one and four may be heteroatoms chosen from N, O, and S (i.e., C₃-C₆ heterocycloalkyl). Examples of lower heterocycloalkyls include pyrrolidinyl, imidazolidinyl, pyrazolidinyl, piperidinyl, piperazinyl, and morpholinyl. Lower heterocycloalkyls may be unsaturated.

The term “lower amino,” as used herein, alone or in combination, refers to —NRR′, wherein R and R′ are independently chosen from hydrogen and lower alkyl, either of which is optionally substituted.

The term “mercaptyl” as used herein, alone or in combination, refers to an RS— group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The term “spirocycle ring system” refers to a polycyclic ring system comprising two rings such that a single atom is common to both rings.

The terms “sulfonate,” “sulfonic acid,” and “sulfonic,” as used herein, alone or in combination, refer the —SO₃H group and its anion as the sulfonic acid is used in salt formation.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —S(O)₂—.

The term “N-sulfonamido” refers to a RS(═O)₂NR′— group with R and R′ as defined herein.

The term “S-sulfonamido” refers to a —S(═O)₂NRR′, group, with R and R′ as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC(S)NR′— group, with R and R′ as defined herein.

The term “O-thiocarbamyl” refers to a —OC(S)NRR′, group with R and R′ as defined herein.

The term “thiocyanato” refers to a —CNS group.

The term “trihalomethanesulfonamido” refers to a X₃CS(O)₂NR— group with X is a halogen and R as defined herein.

The term “trihalomethanesulfonyl” refers to a X₃CS(O)₂— group where X is a halogen.

The term “trihalomethoxy” refers to a X₃CO— group where X is a halogen.

The term “trisubstituted silyl,” as used herein, alone or in combination, refers to a silicone group substituted at its three free valences with groups as listed herein under the definition of substituted amino. Examples include trimethylsilyl, tert-butyldimethylsilyl, triphenylsilyl and the like.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Where structurally feasible, two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety chosen from hydrogen, alkyl, cycloalkyl, heteroalkyl, aryl, heteroaryl and heterocycloalkyl, any of which is optionally substituted. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. For example, an unsymmetrical group such as —C(O)N(R)— may be attached to the parent moiety at either the carbon or the nitrogen.

Asymmetric centers exist in the compounds disclosed herein. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds disclosed herein can exist in unsolvated as well as solvated forms with pharmaceutically acceptable solvents such as water, ethanol, and the like. In general, the solvated forms are considered equivalent to the unsolvated forms.

Certain compounds in the present disclosure contain bicyclo[3.1.0]heptane moieties with substitution in the 7-position. It will be appreciated that two isomers exist for this moiety, which will be termed endo and exo. Geometry of the endo and exo isomers is depicted in the representative structures below:

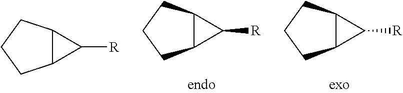

Certain compounds in the present disclosure contain 7-azabicyclo[3.1.0]heptane moieties with substitution in the 7-position. It will be appreciated that two isomers exist for this moiety, which will be termed endo and exo. Geometry of the endo and exo isomers is depicted in the representative structures below:

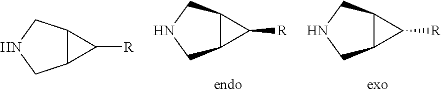

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

A “cognitive disorder,” as used herein refers to a mental health disorder in which loss of cognitive function is the primary symptom, and which primarily affects learning, memory, perception, and/or problem solving. Cognitive disorders include amnesia, dementia, and delirium. Causes may include damage to the memory portions of the brain, whether from trauma or chemotherapy.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

“DLK binder” is used herein to refer to a compound that exhibits an K_{d} with respect to DLK of no more than about 100 μM and more typically not more than about 50 μM, as measured in the DLK binding assay described generally herein. The DLK binding assay measures the K_{d} (dissociation constant) for the binding of a compound with the active site of DLK. Certain compounds disclosed herein have been discovered to bind to DLK. In certain embodiments, compounds will exhibit an K_{d} with respect to DLK of no more than about 10 μM; in further embodiments, compounds will exhibit a K_{d} with respect to DLK of no more than about 1 μM; in yet further embodiments, compounds will exhibit a K_{d} with respect to DLK of not more than about 0.1 μM; in yet further embodiments, compounds will exhibit a K_{d} with respect to DLK of not more than about 10 nM, as measured in the DLK assay described herein.

The phrase “therapeutically effective” is intended to qualify the amount of active ingredients used in the treatment of a disease or disorder or on the effecting of a clinical endpoint.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

As used herein, reference to “treatment” of a patient is intended to include prophylaxis. Treatment may also be preemptive in nature, i.e., it may include prevention of disease. Prevention of a disease may involve complete protection from disease, for example as in the case of prevention of infection with a pathogen, or may involve prevention of disease progression. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease.

The term “patient” is generally synonymous with the term “subject” and includes all mammals including humans. Examples of patients include humans, livestock such as cows, goats, sheep, pigs, and rabbits, and companion animals such as dogs, cats, rabbits, and horses. Preferably, the patient is a human.

The term “prodrug” refers to a compound that is made more active in vivo. Certain compounds disclosed herein may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

The compounds disclosed herein can exist as therapeutically acceptable salts. The present invention includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds disclosed herein, and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

While it may be possible for the compounds of the subject invention to be administered as the raw chemical, it is also possible to present them as a pharmaceutical formulation. Accordingly, provided herein are pharmaceutical formulations which comprise one or more of certain compounds disclosed herein, or one or more pharmaceutically acceptable salts, esters, prodrugs, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. Typically, these methods include the step of bringing into association a compound of the subject invention or a pharmaceutically acceptable salt, ester, amide, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the compounds disclosed herein suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Certain compounds disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

For administration by inhalation, compounds may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

It should be understood that in addition to the ingredients particularly mentioned above, the formulations described above may include other agents conventional in the art having regard to the type of formulation in question, for example those suitable for oral administration may include flavoring agents.

Compounds may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the compounds described herein (or a pharmaceutically acceptable salt, ester, or prodrug thereof) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for diabetes involving administration of one of the compounds described herein, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit.

Specific, non-limiting examples of possible combination therapies include use of certain compounds of the invention with: donepezil, rivastigmine, galantamine, and memantine. Further examples include anti-amyloid antibodies and vaccines, anti-Ab antibodies and vaccines, anti-tau antibodies and vaccines, β-secretase inhibitors, 5-HT4 agonists, 5-HT6 antagonists, 5-HT1a antagonists, α7 nicotinic receptor agonists, 5-HT3 receptor antagonists, PDE4 inhibitors, O-glycnacase inhibitors, and other medicines approved for the treatment of Alzheimer's disease. Further examples include metformin, minocycline, tissue plasminogen activator, and other therapies that improve neuronal survival.

In any case, the multiple therapeutic agents (at least one of which is a compound disclosed herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Thus, in another aspect, certain embodiments provide methods for treating DLK-mediated disorders in a human or animal subject in need of such treatment comprising administering to said subject an amount of a compound disclosed herein effective to reduce or prevent said disorder in the subject, in combination with at least one additional agent for the treatment of said disorder that is known in the art. In a related aspect, certain embodiments provide therapeutic compositions comprising at least one compound disclosed herein in combination with one or more additional agents for the treatment of DLK-mediated disorders.

In certain embodiments, the compounds, compositions, and methods disclosed herein may be useful for the treatment of neurological diseases that result from traumatic injury to central nervous system and peripheral nervous system neurons.

In certain embodiments, the compounds, compositions, and methods disclosed herein may be useful for the treatment of stroke.

In certain embodiments, the compounds, compositions, and methods disclosed herein may be useful for the treatment of traumatic brain injury.

In certain embodiments, the compounds, compositions, and methods disclosed herein may be useful for the treatment of spinal cord injury.

In certain embodiments, the compounds, compositions, and methods disclosed herein may be useful for the treatment of neurologic diseases that result from a chronic neurodegenerative condition.

In certain embodiments, the neurodegenerative condition is Alzheimer's disease.

In certain embodiments, the neurodegenerative condition is frontotemporal dementia.

In certain embodiments, the neurodegenerative condition is Parkinson's disease.

In certain embodiments, the neurodegenerative condition is Huntington's disease.

In certain embodiments, the neurodegenerative condition is amyotrophic lateral sclerosis.

In certain embodiments, the neurodegenerative condition is Alzheimer's disease.

In certain embodiments, the neurodegenerative condition is spinocerebellar ataxia.

In certain embodiments, the neurodegenerative condition is progressive supranuclear palsy.

In certain embodiments, the neurodegenerative condition is Lewy body disease.

In certain embodiments, the neurodegenerative condition is Kennedy's disease.

In certain embodiments, the compounds, compositions, and methods disclosed herein may be useful for the treatment of neuropathies resulting from neural damage.

In certain embodiments, the neuropathy is chemotherapy-induced peripheral neuropathy.

In certain embodiments, the neuropathy is diabetic neuropathy.

In certain embodiments, the compounds, compositions, and methods disclosed herein may be useful for the treatment of cognitive disorders.

In certain embodiments, the cognitive disorder is caused by pharmacological intervention.

In certain embodiments, the cognitive disorder is chemotherapy induced cognitive disorder.

In certain embodiments, the compounds, compositions, and methods disclosed herein may be coadministered with another therapeutic agent.

In certain embodiments, the compounds, compositions, and methods disclosed herein may be coadministered with another therapeutic agent for the treatment of cognitive disorders.

Besides being useful for human treatment, certain compounds and formulations disclosed herein may also be useful for veterinary treatment of companion animals, exotic animals and farm animals, including mammals, rodents, and the like. More preferred animals include horses, dogs, and cats.

### LIST OF ABBREVIATIONS

Ac₂O=acetic anhydride; AcCl=acetyl chloride; AcOH=acetic acid; AIBN=azobisisobutyronitrile; aq.=aqueous; Ar=an aromatic group; BAST=bis(2-methoxyethyl)aminosulfur trifluoride; Bu=butyl; Bu₃SnH=tributyltin hydride; CD₃OD=deuterated methanol; CDCl₃=deuterated chloroform; CDI=1,1′-carbonyldiimidazole; DAST=(diethylamino)sulfur trifluoride; dba=dibenzylideneacetone; DBU=1,8-diazabicyclo[5.4.0]undec-7-ene; DCM=dichloromethane; DEAD=diethyl azodicarboxylate; DIBAL-H=di-iso-butyl aluminium hydride; DIEA=DIPEA=N,N-diisopropylethylamine; DMAP=4-dimethylaminopyridine; DMF=N,N-dimethylformamide; DMSO-d₆=deuterated dimethyl sulfoxide; DMSO=dimethyl sulfoxide; DPPA=diphenylphosphoryl azide; dppf=1,1′-bis(diphenylphosphino)ferrocene; EDC.HCl=EDCI.HCl=1-ethyl-3-(3-dimethylaminopropyl)carbodiimide hydrochloride; Et=ethyl; Et₂O=diethyl ether; EtOAc=ethyl acetate; EtOH=ethanol; h=hour; HATU=2-(1H-7-azabenzotriazol-1-yl)-1,1,3,3-tetramethyl uronium hexafluorophosphate methanaminium; HMDS=hexamethyldisilazane; HOBT=1-hydroxybenzotriazole; i-Pr=isopropyl=2-propyl; i-PrOH=isopropanol; LAH=lithium aluminium hydride; LDA=lithium diisopropyl amide; LiHMDS=Lithium bis(trimethylsilyl)amide; MeCN=acetonitrile; MeI=methyl iodide; MeOH=methanol; MP-carbonate resin=macroporous triethylammonium methylpolystyrene carbonate resin; MsCl=mesyl chloride; MTBE=methyl tertiary butyl ether; n-BuLi=n-butyllithium; NaHMDS=sodium bis(trimethylsilyl)amide; NaOEt=sodium ethoxide; NaOMe=sodium methoxide; NaOtBu=sodium t-butoxide; NBS=N-bromosuccinimide; NCS=N-chlorosuccinimide; NIS=N-iodosuccinimide; NMP=N-Methyl-2-pyrrolidone; Pd(PPh₃)₄=tetrakis(triphenylphosphine)-palladium(0); Pd₂(dba)₃=tris(dibenzylideneacetone)dipalladium(0); PdCl₂(PPh₃)₂=bis(triphenylphosphine)palladium(II) dichloride; PG=protecting group; Ph=phenyl; prep-HPLC=preparative high-performance liquid chromatography; PMB=para-methoxybenzyl; PMBCl=para-methoxybenzyl chloride; PMBOH=para-methoxybenzyl alcohol; PyBop=(benzotriazol-1-yloxy)tripyrrolidinophosphonium hexafluorophosphate; Pyr=pyridine; RT=room temperature; RuPhos=2-dicyclohexylphosphino-2′,6′-diisopropoxybiphenyl; sat.=saturated; ss=saturated solution; tBu=t-Bu=tert-butyl=1,1-dimethylethyl; TBAF=tetrabutylammonium fluoride; TBDPS=t-butyldiphenylsilyl; t-BuOH=tert-butanol; T3P=Propylphosphonic Anhydride; TEA=Et₃N=triethylamine; TFA=trifluoroacetic acid; TFAA=trifluoroacetic anhydride; THF=tetrahydrofuran; TIPS=triisopropylsilyl; Tot=toluene; TsCl=tosyl chloride; Trt=trityl=(triphenyl)methyl; Xantphos=4,5-Bis(diphenylphosphino)-9,9-dimethylxanthene; XPhos=2-dicyclohexylphosphino-2′,4′,6′-triisopropylbiphenyl.

### General Synthetic Methods for Preparing Compounds

The following schemes can be used to practice the present invention.

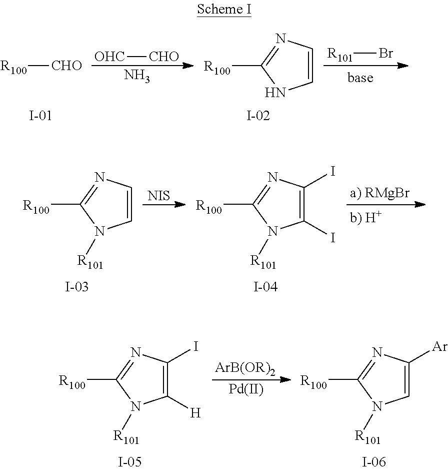

Certain compounds of the present disclosure can be synthesized by using the general synthetic procedure set forth in Scheme I. Formation of imidazole I-02 from aldehyde I-01, glyoxal, and ammonia is followed by amine alkylation, providing I-03. Selective formation of the mono-iodo compound I-05 is accomplished in a two-step procedure: Reaction with two equivalents of NIS gives the 4,5-diiodo compound I-04. Transmetalation with a Grignard reagent takes place selectively at the 5-position, and the resulting organometallic species is quenched with H⁺ to give the 4-iodo compound I-05. The target compound I-06 is obtained by reaction of an arylboronic ester with the iodo-imidazole using well-established coupling techniques.

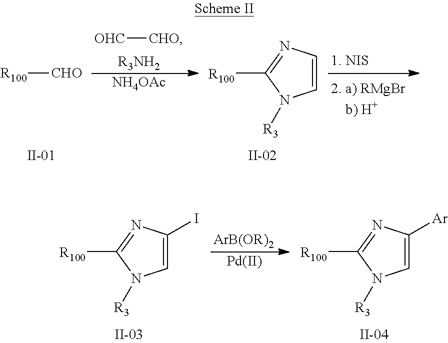

Certain compounds of the present disclosure can be synthesized by using the general synthetic procedure set forth in Scheme II. Formation of disubstituted imidazole II-02 is achieved by reaction of aldehyde II-01, glyoxal, and a substituted primary amine. The monoiodide II-03 is obtained by the same 2-step procedure used in Scheme 1. Finally, a coupling reaction with an arylboronic ester gives the product II-04.

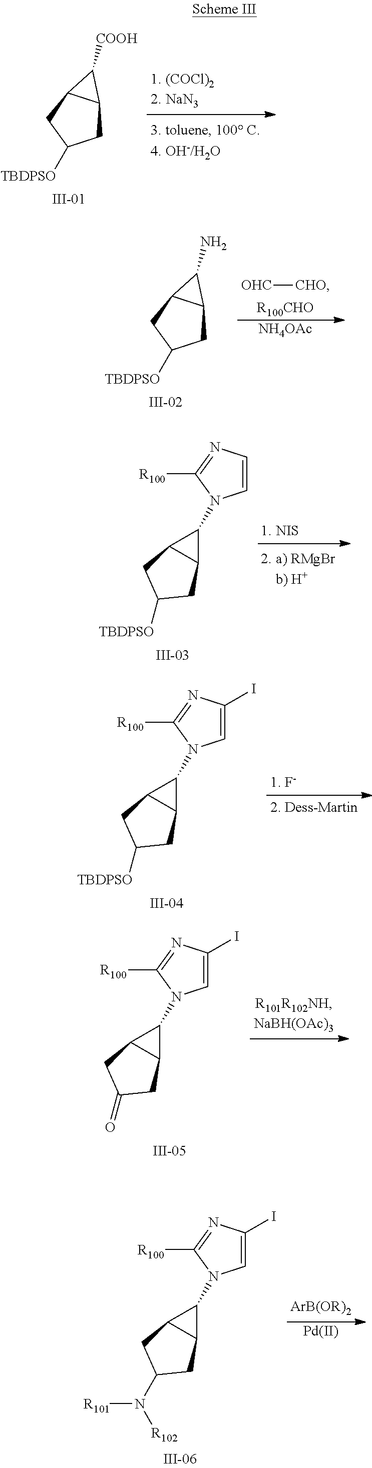

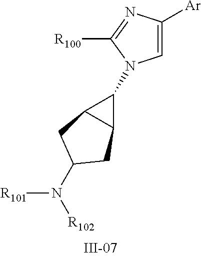

Certain compounds of the present disclosure can be synthesized by using the general synthetic procedure set forth in Scheme III. Using established rearrangement methods, carboxylic acid III-01 is converted via a 4-step sequence to amine III-02. This amine is reacted with glyoxal and a substituted aldehyde to give doubly substituted imidazole III-03. Mono-iodide III-04 is obtained using the two-step procedure introduced in Scheme I. At this point the silyl ether is cleaved using fluoride ion, and the resulting alcohol is oxidized to the carbonyl compound III-05. Reductive amination of the carbonyl compound gives amine III-06. Finally, the iodo functionality is suitable for substitution with an arylboronic ester to give the target compound III-07.

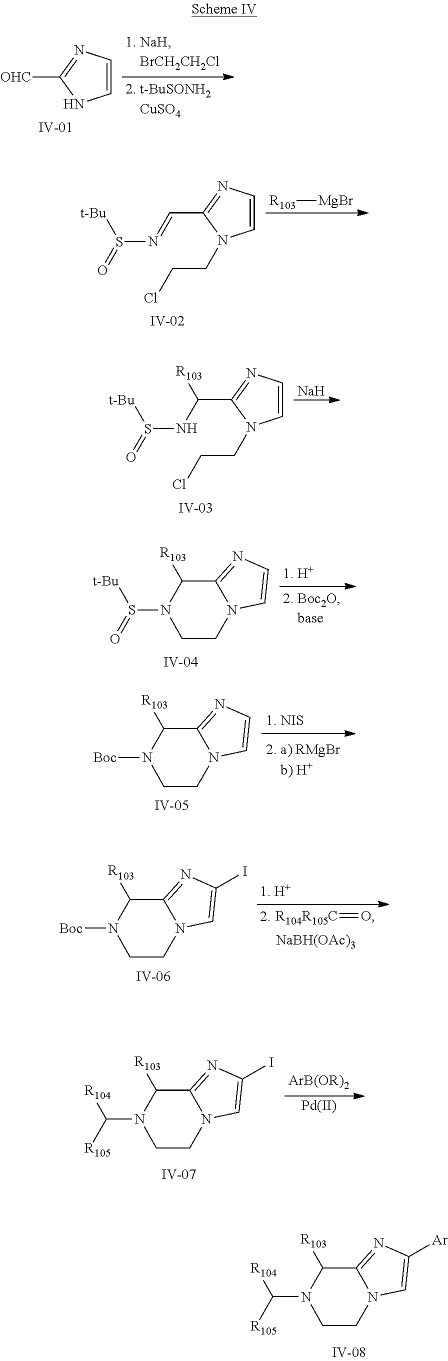

Certain compounds of the present disclosure can be synthesized by using the general synthetic procedure set forth in Scheme IV. Imidazole IV-01 is converted to IV-02 via a two-step procedure consisting of alkylation, followed by condensation with a sulfinamide. The imine functionality is reacted with a Grignard reagent to give IV-03. Ring closure is effected under basic conditions to give the bicyclic compound IV-04. The sulfinamide group is exchanged with a Boc protecting group to give carbamate IV-05. The mono-iodide is obtained by the two-step procedure presented in the schemes above, to give IV-06. The Boc protecting group is removed under acidic conditions, and the newly deprotected amine is condensed under reductive amination conditions to give substituted compound IV-07. Finally, transition-metal promoted coupling gives the product IV-08.

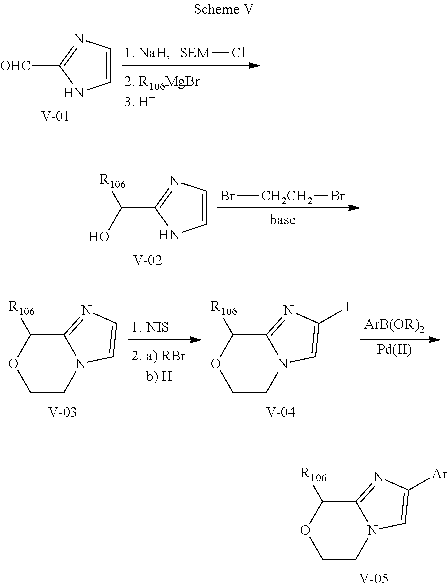

Certain compounds of the present disclosure can be synthesized by using the general synthetic procedure set forth in Scheme V. Aldehyde V-01 is converted to secondary alcohol V-02 via a three-step sequence of amine protection, Grignard reaction, and amine deprotection. Formation of the fused ring structure of V-03 is achieved by alkylation of the amino alcohol with 1,2-dibromoethane. The mono-iodode V-04 is obtained via a 2-step procedure as in the previous examples, and this compound is coupled with an organoboronic acid to give the target compound V-05.

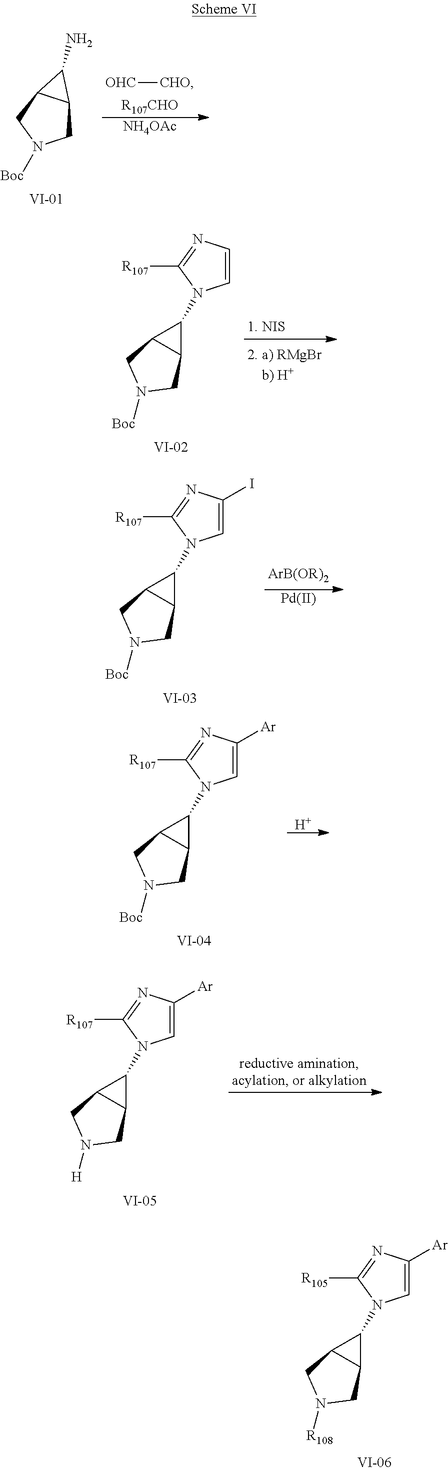

Certain compounds of the present disclosure can be synthesized by using the general synthetic procedure set forth in Scheme VI. Amine VI-01 is converted to disubstituted imidazole VI-02 with glyoxal and an appropriate aldehyde R₂CHO. As disclosed above, selective formation of the mono-iodo compound VI-03 is accomplished in a two-step procedure. Coupling with an arylboronic ester gives trisubstituted imidazole VI-04. The Boc group is removed under acidic conditions to afford secondary amine VI-05, which is available for further elaboration via reductive amination, acylation, or alkylation.

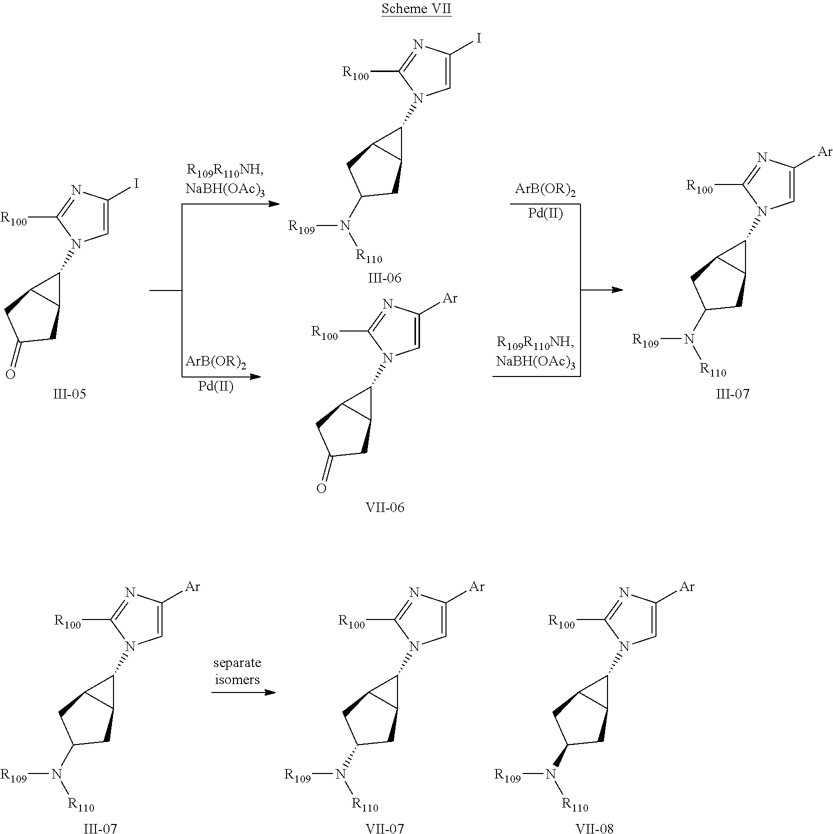

Certain compounds of the present disclosure can be synthesized by using the general synthetic procedure set forth in Scheme VII, which is an elaboration of Scheme III. Synthesis begins with intermediate III-05, which can be converted to amine III-07 via the two-step procedure (reductive amination, followed by arylation) of Scheme III. Alternatively, reversal of the sequence can provide amine III-07 which can be separated to afford isomers VII-07 and VII-08.

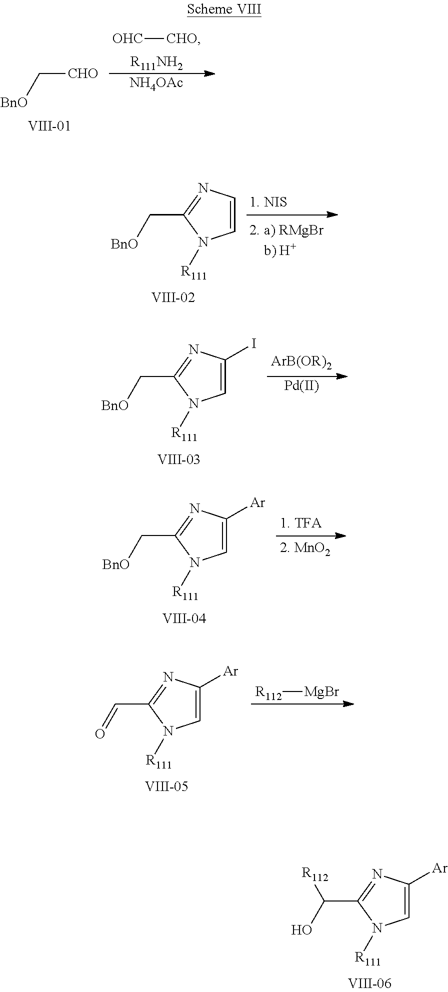

Certain compounds of the present disclosure can be synthesized by using the general synthetic procedure set forth in Scheme VIII. Protected glycolaldehyde VIII-01 is converted to disubstituted imidazole VIII-02, followed by formation of the mono-iodo compound VIII-03 via the two-step procedure disclosed above. Reaction with an arylboronic ester give trisubstituted imidazole VIII-04. Removal of the Bn protecting group can be accomplished under acidic conditions, and the resulting primary alcohol is oxidized to the carboxaldehyde VIII-05. Finally, reaction with a Grignard reagent gives secondary alcohol VIII-06.

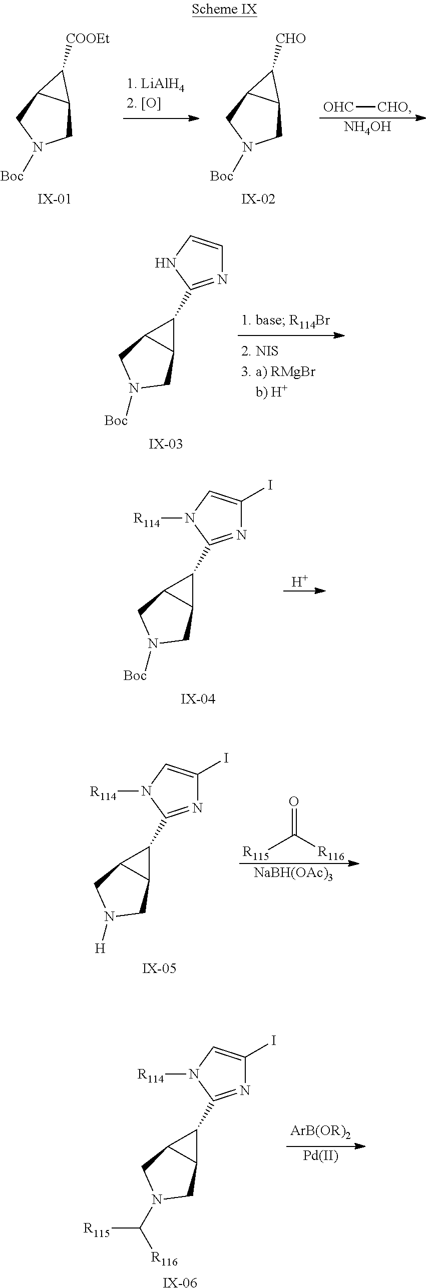

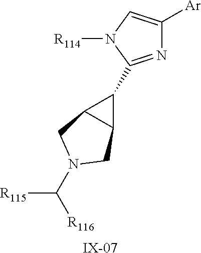

Certain compounds of the present disclosure can be synthesized by using the general synthetic procedure set forth in Scheme IX. Ester IX-01 is converted via reduction/oxidation sequence to carboxaldehyde IX-02. Condensation with glyoxal in the presence of ammonia gives imidazole IX-03. Mono-iodide IX-04 is obtained using the two-step procedure introduced in Scheme I. At this point the Boc group is cleaved using acid to afford secondary amine IX-05. Reaction with a suitable carbonyl compound under reductive amination conditions gives amine IX-06. Finally, the iodo functionality is suitable for substitution with an arylboronic ester to give the target compound IX-07.

### Example 1

### 5-(1-(cyclopropylmethyl)-2-((1R,5S,6r)-3-(oxetan-3-yl)-3-azabicyclo[3.1.0]hexan-6-yl)-1H-imidazol-4-yl)-3-(trifluoromethyl)pyridin-2-amine

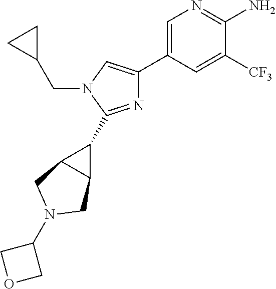 [SMILES: Nc1ncc(-c2cn(CC3CC3)c([C@H]3[C@@H]4CN(C5COC5)C[C@@H]43)n2)cc1C(F)(F)F]

### Step 1: 3-tert-butyl 6-ethyl 3-aza-bicyclo[3.1.0]hexane-3,6-dicarboxylate

To a solution of tert-butyl 2,5-dihydro-1H-pyrrole-1-carboxylate (15.0 g, 88.6 mmol) and Rh₂(OAc)₄ (0.590 g, 1.33 mmol) in CH₂Cl₂ (300 mL) was added dropwise a solution of ethyl diazoacetate (13.05 mL, 124.1 mmol) in CH₂Cl₂ (200 mL) over 60 h. The reaction mixture was filtered and concentrated under reduced pressure. The residue was purified by SiO₂ gel chromatography (0% to 50% EtOAc in petroleum ether) to give the title compound as a light yellow oil (4.8 g, 21%).

¹H NMR (500 MHz, CDCl3) δ 4.13 (q, J=7.1 Hz, 2H), 3.68 (d, J=11.2 Hz, 1H), 3.60 (d, J=11.1 Hz, 1H), 3.41 (t, J=8.8 Hz, 2H), 2.06 (m, 2H), 1.48 (m, 1H), 1.43 (s, 9H), 1.26 (t, J=7.1 Hz, 3H).

### Step 2: (1R,5S,6r)-tert-butyl 6-(hydroxymethyl)-3-azabicyclo[3.1.0]hexane-3-carboxylate

To a solution of the product from the previous step (4.8 g, 19 mmol) in THF (45 mL) was added LiAlH₄ (0.714 g, 18.8 mmol) in portions. The mixture was stirred at RT for 2 h, then treated with 1 M aq. NaOH and extracted with EtOAc (3×45 mL). The combined organic layers were dried over Na₂SO₄ and concentrated under reduced pressure to give the title compound as a yellow oil, which was used without further purification (3.56 g, 89%).

¹H NMR (500 MHz, CDCl₃) δ 3.63-3.53 (m, 3H), 3.49-3.45 (m, 1H), 3.37-3.33 (m, 2H), 1.43-1.41 (m, 12H), 0.95 (m, 1H).

### Step 3: (1R,5S,6r)-tert-butyl 6-formyl-3-azabicyclo[3.1.0]hexane-3-carboxylate

To a solution of the product from the previous step (3.5 g, 16 mmol) in CH₂Cl₂ (150 mL) was added 3,3,3-triacetoxy-3-iodophthalide (10.76 g, 24.62 mmol). The mixture was stirred at RT for 2 h, then concentrated under reduced pressure. The residue was purified by SiO₂ gel chromatography (5:1 petroleum ether:EtOAc) to give the title compound as a white solid (2.05 g, 59%).

MS (ES⁺) C₁₁H₁₇NO₃ requires: 211, found: 234 [M+Na]⁺.

### Step 4: (1R,5S,6r)-tert-butyl 6-(1H-imidazol-2-yl)-3-azabicyclo[3.1.0]hexane-3-carboxylate

To a stirring solution of the product from the previous step (2.03 g, 9.61 mmol) in methanol (30 mL) was added NH₄OH solution (13.36 mL, 96.09 mmol) and glyoxal (0.5325 mL, 10.57 mmol). The mixture was stirred at RT overnight, then concentrated under reduced pressure. The residue was extracted with EtOAc (2×60 mL), and the combined organic layers were dried over Na₂SO₄ and concentrated under reduced pressure. The residue was purified by SiO₂ gel chromatography (0% to 5% MeOH in CH₂Cl₂) to give the title compound as a yellow solid (2.24 g, 94%).

MS (ES⁺) C₁₃H₁₉N₃O₂ requires: 249, found: 250 [M+H]⁺.

### Step 5: (1R,5S,6r)-tert-butyl 6-(1-(cyclopropylmethyl)-1H-imidazol-2-yl)-3-azabicyclo[3.1.0]hexane-3-carboxylate

To a mixture of the product from the previous step (2.24 g, 8.98 mmol) and Cs₂CO₃ (8.87 g, 27.0 mmol) in DMF (15 mL) was added bromomethylcyclopropane (1.31 mL, 13.5 mmol). The mixture was stirred at RT overnight, then poured into water and extracted with EtOAc (3×45 mL). The combined organic layers were washed with sat. aq. NaCl (3×30 mL), dried over Na₂SO₄, and concentrated under reduced pressure. The residue was purified by SiO₂ gel chromatography (0% to 5% MeOH in CH₂Cl₂) to give the title compound as a yellow oil (1.71 g, 63%).

MS (ES⁺) C₁₇H₂₅N₃O₂ requires: 303, found: 304 [M+H]⁺.

### Step 6: (1R,5S,6r)-tert-butyl 6-(1-(cyclopropylmethyl)-4,5-diiodo-1H-imidazol-2-yl)-3-azabicyclo[3.1.0]hexane-3-carboxylate

To a solution of the product from the previous step (1.71 g, 5.64 mmol) in DMF (30 mL) was added NIS (3.42 g, 15.2 mmol). The mixture was stirred at 50° C. for 3 d, then poured into water and extracted with EtOAc (3×50 mL). The combined organic layers were washed with sat. aq. NaCl (4×30 mL), dried over Na₂SO₄, and concentrated under reduced pressure. The residue was purified by SiO₂ gel chromatography (0% to 25% EtOAc in CH₂Cl₂) to give the title compound as a white solid (2.07 g, 66%).

MS (ES⁺) C₁₇H₂₃I₂N₃O₂ requires: 555, found: 556 [M+H]⁺.

### Step 7: (1R,5S,6r)-tert-butyl 6-(1-(cyclopropylmethyl)-4-iodo-1H-imidazol-2-yl)-3-azabicyclo[3.1.0]hexane-3-carboxylate

A solution of the product from the previous step (2.07 g, 3.73 mmol) in THF (20 mL) at −40° C. was added a solution of EtMgBr in Et₂O (3.0 M, 1.74 mL, 5.22 mmol). The mixture was stirred at −40° C. for 30 min, quenched with sat. aq. NH₄Cl, and extracted with EtOAc (2×30 mL). The combined organic layers were washed with sat. aq. NaCl (20 mL), dried over Na₂SO₄, and concentrated under reduced pressure. The residue was purified by SiO₂ chromatography (10% to 45% EtOAc in CH₂Cl₂) to give the title compound as a white solid (0.896 g, 56%).

MS (ES⁺) C₁₇H₂₄IN₃O₂ requires: 429, found: 430 [M+H]⁺.

### Step 8: (1R,5S,6r)-6-(1-(cyclopropylmethyl)-4-iodo-1H-imidazol-2-yl)-3-azabicyclo[3.1.0]hexane

To a solution of the product from the previous step (0.425 g, 0.990 mmol) in CH₂Cl₂ (5 mL) was added TFA (1.0 mL, 13 mmol), and the mixture was stirred at RT for 2 h then concentrated under reduced pressure to give the title compound as an oil, which was used without further purification (0.320 g, 98%).

MS (ES⁺) C₁₂H₁₆IN₃ requires: 329, found: 330 [M+H]⁺.

### Step 9: (1R,5S,6r)-6-(1-(cyclopropylmethyl)-4-iodo-1H-imidazol-2-yl)-3-(oxetan-3-yl)-3-azabicyclo[3.1.0]hexane

To a solution of the product from the previous step (0.320 g, 0.972 mmol) in MeOH (5 mL) was added 3-oxetanone (0.350 g, 4.86 mmol), and the mixture was stirred at RT for 1 h. To the mixture was added NaCNBH₃ (0.0611 g, 0.972 mmol). The mixture was stirred at RT overnight, then concentrated under reduced pressure. The residue was purified by SiO₂ gel chromatography (25% to 100% EtOAc in petroleum ether) to give the title compound as a white solid (0.356 g, 95%).

MS (ES⁺) C₁₅H₂₀IN₃O requires: 385, found: 386 [M+H]₊.

### Step 10: 5-(1-(cyclopropylmethyl)-2-((1R,5S,6r)-3-(oxetan-3-yl)-3-azabicyclo[3.1.0]hexan-6-yl)-1H-imidazol-4-yl)-3-(trifluoromethyl)pyridin-2-amine

A mixture of the product from the previous step (75.0 mg, 195 mmol), 5-(4,4,5,5-tetramethyl-1,3,2-dioxaborolan-2-yl)-3-(trifluoromethyl)pyridin-2-amine (84.1 mg, 292 mmol), Cs₂CO₃ (190.3 mg, 584.0 mmol) and Fe(dppf)Cl₂ (16.2 mg, 19.5 mmol) in 5:1 dioxane:water (5 mL) was degassed and purged with N₂, then stirred at 100° C. for 2 h. The mixture was concentrated under reduced pressure, and the residue was purified by SiO₂ gel chromatography to give the title compound as a light brown solid (30.0 mg, 37%).

MS (ES⁺) C₂₁H₂₄F₃N₅O requires: 419, found: 420 [M+H]⁺.

¹H NMR (500 MHz, CDCl₃) δ 8.54 (appar s, 1H), 8.07 (appar s, 1H), 7.13 (s, 1H), 4.90 (s, 2H), 4.70 (appar t, J=6.6 Hz, 2H), 4.62 (appar t, J=6.1 Hz, 2H), 3.85 (d, J=6.9 Hz, 2H), 3.79 (dd, J=12.5, 6.3 Hz, 1H), 3.15 (d, J=8.8 Hz, 2H), 2.50 (d, J=8.5 Hz, 2H), 2.28 (d, J=2.9 Hz, 1H), 2.13 (s, 2H), 1.27-1.23 (m, 1H), 0.70 (appar q, J=5.6 Hz, 2H), 0.41 (appar q, J=5.1 Hz, 2H).

### Example 2

### 5-(2-(cyclopropylmethyl)-1-((1R,5S,6s)-3-(oxetan-3-yl)-3-azabicyclo[3.1.0]hexan-6-yl)-1H-imidazol-4-yl)-3-(trifluoromethyl)pyridin-2-amine

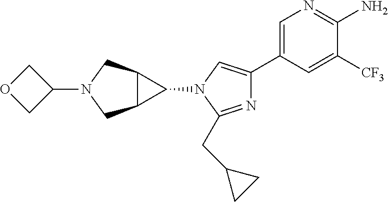 [SMILES: Nc1ncc(-c2cn([C@H]3[C@@H]4CN(C5COC5)C[C@@H]43)c(CC3CC3)n2)cc1C(F)(F)F]

### Step 1: tert-butyl (1R,5S,6s)-6-(2-(cyclopropylmethyl)-1H-imidazol-1-yl)-3-aza-bicyclo[3.1.0]hexane-3-carboxylate

To a solution of 2-cyclopropylacetaldehyde (170 mg, 1.01 mmol) in MeOH (1 mL) was added (1R,5S,6s)-tert-butyl 6-amino-3-azabicyclo[3.1.0]hexane-3-carboxylate (200 mg, 1.01 mmol) in MeOH (1 mL) dropwise, then ammonium acetate (78 mg, 1.01 mmol) in MeOH (1 mL). To the mixture was then added glyoxal (146 mg, 1.01 mmol) dropwise, and the reaction was stirred at RT for 24 h. The mixture was diluted with EtOAc (20 mL) then washed with sat. aq. NaHCO₃, dried over Na₂SO₄, filtered and concentrated under reduced pressure. The residue was purified by SiO₂ gel chromatography (two purifications: 60% to 100% EtOAc in hexanes, then 0% to 40% MeOH in EtOAc) to give the title compound as a colorless liquid (110 mg, 36%).

MS (ES⁺) C₁₇H₂₅N₃O₂ requires: 303, found: 304 [M+H]⁺.

### Step 2: tert-butyl (1R,5S,6s)-6-(2-(cyclopropylmethyl)-4,5-diiodo-M-imidazol-1-yl)-3-azabicyclo[3.1.0]hexane-3-carboxylate

A solution of the product from the previous step (97 mg, 0.32 mmol) and NIS (180 mg, 0.799 mmol) in DMF (2 ml) was stirred at 80° C. for 2 h, then treated with sat. aq. Na₂S₂O₃ and stirred at RT for 1 h. The mixture was partitioned between EtOAc and water, and the organic layer was concentrated under reduced pressure. The residue was purified by SiO₂ gel chromatography (10% to 100% EtOAc in hexanes) to give the title compound as a brown liquid (118 mg, 66%).

MS (ES⁺) C₁₇H₂₃I₂N₃O₂ requires: 555, found: 556 [M+H]⁺.

### Step 3: tert-butyl (1R,5S,6s)-6-(2-(cyclopropylmethyl)-4-iodo-M-imidazol-1-yl)-3-azabicyclo[3.1.0]hexane-3-carboxylate

To a solution of the product from the previous step (275 mg, 0.495 mmol) in THF (4 ml) at −40° C. was added isopropylmagnesium chloride in THF (2.0 M, 0.322 ml, 0.644 mmol). The mixture was allowed to warm to 0° C., then treated with AcOH (0.5 mL), diluted with EtOAc, and washed with sat. aq. Na₂CO₃. The separated organic layer was sequentially washed with water then sat. aq. NaCl, dried over Na₂SO₄, and concentrated under reduced pressure. The residue was purified by SiO₂ gel chromatography (0% to 50% EtOAc in hexanes) to give the title compound as a white solid (151 mg, 71%).

MS (ES⁺) C₁₇H₂₄IN₃O₂ requires: 429, found: 430 [M+H]⁺.

### Step 4: (1R,5S,6s)-6-(2-(cyclopropylmethyl)-4-iodo-M-imidazol-1-yl)-3-azabicyclo[3.1.0]hexane

A mixture of the product from the previous step (0.215 g, 0.5 mmol) in TFA (2 mL) and CH₂Cl₂ (2 mL) was stirred for 30 min, then concentrated under reduced pressure. The residue was partitioned between THF and sat. aq. NaHCO₃, and the organic layer was dried over Na₂SO₄ and concentrated under reduced pressure to give the crude title compound, which was used without further purification in the next step.

MS (ES⁺) C₁₂H₁₆IN₃ requires: 329, found: 330 [M+H]⁺.

### Step 5: (1R,5S,6s)-6-(2-(cyclopropylmethyl)-4-iodo-1H-imidazol-1-yl)-3-(oxetan-3-yl)-3-azabicyclo[3.1.0]hexane

To a solution of the crude product from the previous step (theoretical 0.5 mmol) in CH₂Cl₂ (5 ml) was added oxetan-3-one (180 mg, 2.50 mmol), and the resulting mixture was stirred at RT for 0.5 h, then treated with NaBH(OAc)₃ (530 mg, 2.50 mmol) in 4 portions at a time interval of 10 min. Water (100 mL) was added to the mixture, and layers were separated. The aqueous layer was extracted with CH₂Cl₂ (3×50 mL), and the combined organic layers were washed with sat. aq. NaCl, dried over MgSO4, and concentrated under reduced pressure. The residue was purified by SiO₂ gel chromatography (0% to 3% MeOH in CH₂Cl₂) to give the title compound as a pale yellow solid (120 mg, 62%).

MS (ES⁺) C₁₅H₂₀IN₃O requires: 385, found: 386 [M+H]⁺.

### Step 6: 5-(2-(cyclopropylmethyl)-1-((1R,5S,6s)-3-(oxetan-3-yl)-3-azabicyclo[3.1.0]hexan-6-yl)-1H-imidazol-4-yl)-3-(trifluoromethyl)pyridin-2-amine

A mixture of the product from the previous step (12 mg, 0.032 mmol), 5-(4,4,5,5-tetramethyl-1,3,2-dioxaborolan-2-yl)-3-(trifluoromethyl)pyridin-2-amine (15.44 mg, 0.054 mmol), PdCl₂(dppf)-CH₂Cl₂Adduct (6.56 mg, 8.04 μmol) and K₂CO₃ (0.080 ml, 0.161 mmol) in DMF (0.5 ml) was degassed by three times evacuating the flask and back-filling with nitrogen at RT. The reaction mixture was stirred at 90° C. for 1 h. The mixture was filtered through cotton and purified by reverse phase preparative HPLC (Mobile phase: A=0.1% TFA/H₂O, B=0.1% TFA/MeCN; Gradient: B=10-30%; 30 min; Column: C18) to give the title compound as a presumed trifluoroacetate salt, as a white solid (5.3 mg, 39% yield).

MS (ES⁺) C₂₁H₂₄F₃N₅O requires: 419, found: 420 [M+H]⁺.

¹H NMR (600 MHz, CD₃OD-d₄) δ 8.49 (d, J=2.21 Hz, 1H), 8.16 (d, J=2.21 Hz, 1H), 7.85 (s, 1H), 4.88 (t, J=7.33 Hz, 2H), 4.77 (dd, J=4.81, 7.77 Hz, 2H), 4.40-4.50 (m, 1H), 3.97 (t, J=2.33 Hz, 1H), 3.92 (d, J=11.44 Hz, 2H), 3.58 (d, J=12.21 Hz, 2H), 3.03 (d, J=7.26 Hz, 2H), 2.79 (dd, J=4.41, 2.47 Hz, 2H), 1.19 (m, 1H), 0.76 (m, 2H), 0.43 (m, 2H).

### Example 3

### 5-(2-isopropyl-1-((1R,5S,6r)-3-morpholinobicyclo[3.1.0]hexan-6-yl)-1H-imidazol-4-yl)-3-(trifluoromethyl)pyridin-2-amine

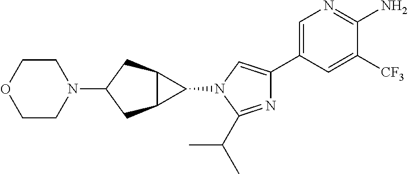

### Step 1: (1R,5S,6r)-3-((tert-butyldiphenylsilyl)oxy)bicyclo[3.1.0]hexan-6-amine

To a stirring suspension of (1R,5S,6r)-3-((tert-butyldiphenylsilyl)oxy)bicyclo-[3.1.0]hexane-6-carboxylic acid (350 mg, 0.920 mmol) and DMF (2.1 μl, 0.028 mmol) in CH₂Cl₂ (5 mL) was added oxalyl chloride (0.322 mL, 3.68 mmol) dropwise. The mixture was stirred at RT for 1 h, then concentrated under reduced pressure, treated with toluene (1 mL) and again concentrated under reduced pressure. The residue was dissolved again in toluene (3 mL). To the stirring solution at 0° C. was added dropwise a solution of NaHCO₃ (0.098 g, 0.92 mmol), NaN₃ (0.179 g, 2.76 mmol), and BuN₄Br (0.059 g, 0.18 mmol) in water. The mixture was stirred at 0° C. for 3 h. The layers were separated and the organic layer was sequentially washed with cold water (3 mL) then cold 20% aq. NaCl (3 mL), dried over Na₂SO₄ and filtered, using 3 mL of toluene in rinsing. The toluene solution was heated to 100° C. and stirred for 4 h, then concentrated under reduced pressure. The residue was treated with THF (3 mL) and aqueous NaOH (0.5 M, 2.76 mL, 1.38 mmol), and the mixture was stirred at RT for 10 min. The mixture was then diluted with EtOAc, washed with sat. aq. NaHCO₃, and concentrated under reduced pressure. The residue was purified via SiO₂ gel chromatography (0% to 15% MeOH in CH₂Cl₂) to give the title compound as a colorless liquid (125 mg, 39% yield).

MS (ES⁺) C₂₂H₂₉NOSi requires: 351, found: 352 [M+H]⁺.

### Step 2: 1-((1R,5S,6r)-3-((tert-butyldiphenylsilyl)oxy)bicyclo[3.1.0]hexan-6-yl)-2-isopropyl-1H-imidazole

To a solution of isobutyraldehyde (20.5 mg, 0.284 mmol) in MeOH (1 mL) was added a solution of the product from the previous step (100 mg, 0.284 mmol) in MeOH (1 mL) dropwise, followed by a solution of NH₄OAc (21.9 mg, 0.284 mmol) in MeOH (1 mL). To the mixture was added glyoxal (41.3 mg, 0.284 mmol) dropwise, and the mixture was stirred at RT for 24 h then diluted with EtOAc (20 mL) and washed with sat. aq. NaHCO₃. The organic layer was dried over Na₂SO₄ and concentrated under reduced pressure. The residue was purified via SiO₂ gel chromatography (two purifications: 60% to 100% EtOAc in hexanes then 0% to 40% MeOH in EtOAc) to give the title compound as a colorless liquid (50 mg, 40%).

MS (ES⁺) C₂₈H₃₆N₂OSi requires: 444, found: 445 [M+H]⁺.

### Step 3: 1-((1R,5S,6r)-3-((tert-butyldiphenylsilyl)oxy)bicyclo[3.1.0]hexan-6-yl)-4,5-diiodo-2-isopropyl-1H-imidazole

A solution of the product from the previous step (1121 mg, 2.520 mmol) and NIS (1701 mg, 7.560 mmol) in DMF (2 ml) was stirred at 80° C. for 30 min, then treated with sat. aq. Na₂S₂O₃ and rapidly stirred at RT for 30 min. The mixture was partitioned between EtOAc and water, and the organic layer was concentrated under reduced pressure. The residue was purified via SiO₂ gel chromatography (10% to 60% EtOAc in hexanes) to give the title compound as a colorless liquid (550 mg, 31%).

MS (ES⁺) C₂₈H₃₄I₂N₂OSi requires: 696, found: 697 [M+H]⁺.

### Step 4: 1-((1R,5S,6r)-3-((tert-butyldiphenylsilyl)oxy)bicyclo[3.1.0]hexan-6-yl)-4-iodo-2-isopropyl-1H-imidazole

To a solution of the product from the previous step (250 mg, 0.359 mmol) in THF (1 ml) at −40° C. was added dropwise a solution of isopropylmagnesium chloride in THF (2.0 M, 0.233 ml, 0.467 mmol). The mixture was allowed to warm to 0° C., then treated with AcOH (0.5 mL), diluted with EtOAc, and washed with sat. aq. NaHCO₃. The layers were separated, and the organic layer was sequentially washed with water then sat. aq. NaCl, dried over Na₂SO₄ and concentrated under reduced pressure. The residue was purified by SiO₂ gel chromatography (0% to 50% EtOAc in hexanes) to give the title compound as a white solid (151 mg, 74%).

MS (ES⁺) C₂₈H₃₅IN₂OSi requires: 570, found: 571 [M+H]⁺.

### Step 5: (1R,5S,6r)-6-(4-iodo-2-isopropyl-1H-imidazol-1-yl)bicyclo[3.1.0]hexan-3-ol

A mixture of the product from the previous step (80 mg, 0.14 mmol) and TBAF in THF (1.0 M, 0.421 ml, 0.421 mmol) in THF (1.4 mL) was stirred at RT for 2 h. The mixture was then treated with sat. aq. NaHCO₃ and extracted with EtOAc. The organic layer was dried over Na₂SO₄, filtered and concentrated under reduced pressure to give the crude title compound, which was used in the next step without further purification.

MS (ES⁺) C₁₂H₁₇IN₂O requires: 332, found: 333 [M+H]⁺.

### Step 6: (1R,5S,6r)-6-(4-iodo-2-isopropyl-1H-imidazol-1-yl)bicyclo[3.1.0]hexan-3-one

To a solution of the product from the previous step (40.0 mg, 0.120 mmol) in CH₂Cl₂ (1.2 mL) was added Dess-Martin periodinane (102 mg, 0.241 mmol). The mixture was stirred at RT for 2 h, then treated with MeOH (1 mL), stirred for 30 min, and concentrated under reduced pressure. The residue was purified by SiO₂ gel chromatography (0% to 20% MeOH in CH₂Cl₂ to give the title compound as a white solid (38 mg, 96%).

MS (ES⁺) C₁₂H₁₅IN₂O requires: 330, found: 331 [M+H]⁺.

### Step 7: 4-((1R,5S,6r)-6-(4-iodo-2-isopropyl-1H-imidazol-1-yl)bicyclo[3.1.0]hexan-3-yl)morpholine

To a mixture of morpholine (0.100 ml, 1.15 mmol) and the product from the previous step (38.0 mg, 0.115 mmol) in 1,2-dichloroethane (1.5 ml) was added AcOH (0.020 ml, 0.34 mmol). The mixture was stirred for 30 min, then treated with NaBH(OAc)₃ (195 mg, 0.921 mmol) and rapidly stirred at RT for 8 h. The mixture was then added to a premixed solution of aq. conc. HCl (1 mL) in MeOH (15 mL), and the new mixture was concentrated under reduced pressure. The residue was purified by SiO₂ gel chromatography (0% to 20% MeOH in CH₂Cl₂) to give the title compound as a colorless film (40 mg, 87%).

MS (ES⁺) C₁₆H₂₄IN₃O requires: 401, found: 402 [M+H]⁺.

### Step 8: 5-(2-isopropyl-1-((1R,5S,6r)-3-morpholinobicyclo[3.1.0]hexan-6-yl)-1H-imidazol-4-yl)-3-(trifluoromethyl)pyridin-2-amine

A mixture of the product from the previous step (10 mg, 0.025 mmol), 5-(4,4,5,5-tetramethyl-1,3,2-dioxaborolan-2-yl)-3-(trifluoromethyl)pyridin-2-amine (11.96 mg, 0.042 mmol), PdCl₂(dppf)-CH₂Cl₂ adduct (5.09 mg, 6.23 μmol) and K₂CO₃ (0.062 ml, 0.125 mmol) in DMF (0.4 ml) was degassed by three times evacuating the flask and back-filling with nitrogen at RT. The reaction mixture was stirred at 90° C. for 1 h. The mixture was filtered through cotton and purified by reverse phase preparative HPLC (Mobile phase: A=0.1% TFA/H₂O, B=0.1% TFA/MeCN; Gradient: B=10-30%; 30 min; Column: C18) to give the title compound as a presumed trifluoroacetate salt as a white solid (4.3 mg, 40% yield).

MS (ES⁺) C₂₂H₂₈F₃N₅O requires: 435, found: 436 [M+H]⁺.

¹H NMR (600 MHz, CD₃OD-d₄) δ 8.26 (s, 1H), 7.90 (s, 1H), 7.75 (s, 1H), 4.15-3.97 (m, 3H), 3.77-3.88 (m, 2H), 3.59-3.64 (m, 1H), 3.40-3.56 (m, 3H), 3.05-3.22 (m, 2H), 2.62-2.87 (m, 2H), 1.92-2.38 (m, 4H), 1.48 (m, 6H). The NMR spectrum suggests a 1:2 mixture of cis/trans isomers.

### Example 4

### 5-[8-Cyclopropyl-7-(oxetan-3-yl)-5H,6H,7H,8H-imidazo[1,2-a]pyrazin-2-yl]-3-(trifluoromethyl)pyridin-2-amine

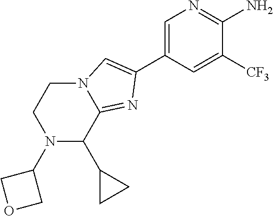 [SMILES: Nc1ncc(-c2cn3c(n2)C(C2CC2)N(C2COC2)CC3)cc1C(F)(F)F]

### Step 1: 1-(2-chloroethyl)-1H-imidazole-2-carbaldehyde

To a suspension of NaH (60% in mineral oil, 234 mg, 5.85 mmol) in DMF (20 ml) at 0-5° C. was added 1H-imidazole-2-carbaldehyde (500 mg, 5.21 mmol) in portions over 10 sec, and the resulting off-white mixture was stirred at RT for 1.5 h, in which time it becomes a cloudy yellow solution. To the solution was added 1-bromo-2-chloroethane (0.480 ml, 5.77 mmol) over 45 sec, and the resulting cloudy yellow solution was stirred at RT for 14 h. Water (0.5 mL) was added, and the mixture was concentrated under reduced pressure to an oily tan solid. The residue was purified by SiO₂ gel chromatography (0% to 50% EtOAc in hexanes) to give the title compound as a colorless oil (549 mg, 66%).

MS (ES⁺) C₆H₇ClN₂O requires: 158, found: 159 [M+H]⁺.

### Step 2: (E)-N-((1-(2-chloroethyl)-1H-imidazol-2-yl)methylene)-2-methylpropane-2-sulfinamide

To a solution of the product from the previous step (539 mg, 3.40 mmol) in CH₂Cl₂ (7 ml) were added 2-methylpropane-2-sulfinamide (379 mg, 3.12 mmol) and CuSO₄ (991 mg, 6.21 mmol) and the resulting greenish-blue suspension was stirred at RT for 16.5 h. The blue-green suspension was filtered thru Celite 545® and concentrated under reduced pressure to a yellow-green oil. The residue was purified by SiO₂ gel chromatography (0% to 50% EtOAc in hexanes) to give the title compound as a white solid (688 mg, 84%).

MS (ES⁺) C₁₀H₁₆ClN₃OS requires: 261, found: 262 [M+H]⁺.

### Step 3: N-((1-(2-chloroethyl)-1H-imidazol-2-yl)(cyclopropyl)methyl)-2-methylpropane-2-sulfinamide

To a solution of the product from the previous step (484 mg, 1.85 mmol) in THF (18.5 mL) at −78° C. was added cyclopropylmagnesium bromide in 2-methyltetrahydrofuran (2.0 M, 3.7 mL, 7.4 mmol) all at once. The pale yellow solution was allowed to quickly warm to RT and then stirred for 16 h. To the solution was added sat. aq. NH₄Cl (20 mL), and the resulting mixture was partitioned between water (20 mL) and CH₂Cl₂ (40 mL). The aqueous layer was further extracted with CH₂Cl₂ (2×20 mL), and the three combined organic layers were dried over Na₂SO₄, filtered and concentrated under reduced pressure to give the crude title compound as a pale yellow solid, which was used without further purification (626 mg, 111% crude yield).

MS (ES⁺) C₁₃H₂₂ClN₃OS requires: 303, found: 304 [M+H]⁺.

### Step 4: tert-butyl 8-cyclopropyl-5,6-dihydroimidazo[1,2-c]pyrazine-7(8H)-carboxylate

To a solution of the crude product from the previous step (610 mg, 2.01 mmol) in DMF (20 ml) at 0-5° C. was added NaH (60% in mineral oil, 205 mg, 5.13 mmol) and the resulting pale yellow mixture, initially bubbling, was stirred at 0-5° C. for 5 min then RT for 2.5 h. To the orange mixture was added water (0.5 mL, bubbling observed), and the mixture was then concentrated under reduced pressure. The residue was purified by SiO₂ gel chromatography (0% to 5% MeOH in EtOAc) to give 426 mg of a pale yellow solid. MS (ES⁺) C₁₃H₂₁N₃OS requires: 267, found: 268 [M+H]⁺. While only one peak is apparent by chromatography, the NMR spectrum is consistent with a mixture of 7-(tert-butylsulfinyl)-8-cyclopropyl-5,6,7,8-tetrahydroimidazo[1,2-a]pyrazine and N-(cyclopropyl(1-vinyl-1H-imidazol-2-yl)methyl)-2-methylpropane-2-sulfinamide.

To the mixture was added a premixed solution of methanol (16 ml) and acetyl chloride (4 ml), and the yellow solution was stirred at RT for 3 h then concentrated to a pale orange residue. To the residue was added methanol (16 mL), N-ethyl-N-isopropylpropan-2-amine (0.835 ml, 4.78 mmol) and di-tert-butyl dicarbonate (349 mg, 1.60 mmol). The yellow solution was stirred for 1 h, then concentrated under reduced pressure to an orange oil. The residue was purified by SiO₂ gel chromatography (0% to 80% EtOAc in hexanes) to give the title compound as a pale yellow oil, contaminated with some aliphatic impurities, which was used without further purification (131 mg, 31%).

MS (ES⁺) C₁₄H₂₁N₃O₂ requires: 263, found: 264 [M+H]⁺.

### Step 5: tert-butyl 8-cyclopropyl-2,3-diiodo-5,6-dihydroimidazo[1,2-a]pyrazine-7(8H)-carboxylate

To a solution of the impure product from the previous step (88 mg, 0.33 mmol) in DMF (2 ml) was added NIS (192.8 mg, 0.857 mmol), and the resulting pale yellow solution, which quickly turns orange, was stirred at 50° C. for 28 h. The orange solution was allowed to cool, then treated with sat. aq. sodium thiosulfate (0.5 mL). The resulting pale yellow mixture was concentrated under reduced pressure to a yellow residue. The residue was purified by SiO₂ gel chromatography (0% to 20% EtOAc in hexanes) to give the title compound as a white solid (131 mg, 76%).

MS (ES⁺) C₁₄H₁₉I₂N₃O₂ requires: 515, found: 516 [M+H]+

### Step 6: tert-butyl 8-cyclopropyl-2-iodo-5,6-dihydroimidazo[1,2-a]pyrazine-7(8H)-carboxylate

To a solution of product from the previous step (79.8 mg, 0.155 mmol) in THF (6 ml) at 0-5° C. was added EtMgBr in ether (3.0 M, 0.055 ml, 0.16 mmol) all at once, and the resulting colorless solution was stirred at 0-5° C. for 35 min. The solution was treated with sat. aq. NH₄Cl (1 mL), and the resulting yellow mixture was allowed to warm to RT then partitioned between water (5 mL) and EtOAc (10 mL). The aqueous layer was extracted with EtOAc (10 mL), and the combined organic layers were dried over Na₂SO₄, filtered and concentrated under reduced pressure to give the crude title compound as a yellow oil, which was used without further purification (58.7 mg, 97%).

MS (ES⁺) C₁₄H₂₀IN₃O₂ requires: 389, found: 390 [M+H]⁺.

### Step 7: 8-cyclopropyl-2-iodo-7-(oxetan-3-yl)-5,6,7,8-tetrahydroimidazo[1,2-a]pyrazine

To the crude product from the previous step (58.7 mg, 0.151 mmol) was added CH₂Cl₂ (1 ml) and TFA (1 mL), and the orange solution was stirred at RT for 30 min. The solution was concentrated under reduced pressure, treated with 1 mL of toluene, and again concentrated under reduced pressure to an orange residue. To the residue was added DCE (1.5 ml) and oxetan-3-one (0.013 mL, 0.20 mmol). The orange mixture was stirred at RT for 15 min, then NaBH(OAc)₃ (48.9 mg, 0.231 mmol) was added and the resulting orange mixture stirred at RT for 18.5 h, in which time it turns yellow. To the mixture was added additional oxetan-3-one (0.013 μl, 0.20 mmol) and NaBH(OAc)₃ (49.6 mg, 0.263 mmol). The yellow solution was stirred at RT for 2 h, then partitioned between CH₂Cl₂ (10 mL) and 2.0 M aq. NaOH (5 mL). The aqueous layer was extracted with CH₂Cl₂ (10 mL), and the two combined organic layers were dried over Na₂SO₄, filtered and concentrated under reduced pressure to a yellow residue. The residue was purified via SiO₂ gel chromatography (0% to 1% MeOH in EtOAc) to give the title compound as a white solid (24.8 mg, 48%).

MS (ES⁺) C₁₂H₁₆IN₃O requires: 345, found: 346 [M+H]⁺.

### Step 8: 5-(8-cyclopropyl-7-(oxetan-3-yl)-5,6,7,8-tetrahydroimidazo[1,2-a]pyrazin-2-yl)-3-(trifluoromethyl)pyridin-2-amine

To a solution of the product from the previous step (27 mg, 0.093 mmol) and PdCl₂(dppf)-CH₂Cl₂ (5.7 mg, 0.0070 mmol) in DMF (1 ml) was added K₂CO₃ in water (2.0 M, 0.104 ml, 0.208 mmol). The orange-yellow mixture, containing a small amount of undissolved white solid, was degassed by bubbling nitrogen through it via a needle for 1 min. The mixture was then stirred at 90° C. for 12 h. The resulting dark yellow mixture was allowed to cool, then concentrated under reduced pressure to a dark yellow residue. The residue was purified by SiO₂ gel chromatography (0% to 5% MeOH in EtOAc) to give the title compound as a yellow solid (16 mg, 62%).

MS (ES⁺) C₁₈H₂₀F₃N₅O requires: 379, found: 380 [M+H]⁺.

¹H NMR (600 MHz, DMSO-d₆) δ 8.56 (d, J=1.89 Hz, 1H), 7.98 (d, J=2.27 Hz, 1H), 7.51 (s, 1H), 6.36 (s, 2H), 4.58-4.65 (m, 2H), 4.46-4.55 (m, 2H), 4.12 (appar t, J=6.61 Hz, 1H), 3.89-4.00 (m, 2H), 3.26 (appar dt, J=4.91, 8.88 Hz, 1H), 3.21 (appar d, J=8.31 Hz, 1H), 2.92-2.98 (m, 1H), 1.03-1.11 (m, 1H), 0.40-0.54 (m, 3H), 0.32-0.40 (m, 1H).

### Example 5

### 5-(8-methyl-6,8-dihydro-5H-imidazo[2,1-c][1,4]oxazin-2-yl)-3-(trifluoromethyl)pyridin-2-amine

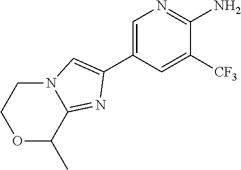 [SMILES: CC1OCCn2cc(-c3cnc(N)c(C(F)(F)F)c3)nc21]

### Step 1: 1-((2-(trimethylsilyl)ethoxy)methyl)-1H-imidazole-2-carbaldehyde

To a suspension of NaH (60% in mineral oil, 1.45 g, 36.2 mmol) in DMF (30 mL) was added 2-imidazolecarboxaldehyde (3.00 g, 30.3 mmol) portionwise, and the mixture was stirred at RT for 1 h, then treated with 2-(trimethylsilyl)ethoxymethyl chloride (5.91 mL, 33.3 mmol). The mixture was stirred at RT overnight, then treated with sat. aq. NH₄Cl and extracted with EtOAc (3×45 mL). The combined organic layers were washed with sat. aq. NaCl (6×30 mL), dried over Na₂SO₄, and concentrated under reduced pressure. The residue was purified by SiO₂ gel chromatography (0% to 25% EtOAc in CH₂Cl₂) to give the title compound as a colorless oil (2.18 g, 32%).

¹H NMR (400 MHz, CDCl₃) δ 9.86 (s, 1H), 7.39 (s, 1H), 7.36 (s, 1H), 5.80 (s, 2H), 3.66-3.47 (m, 2H), 0.98-0.90 (m, 2H), 0.01 (s, 9H).

### Step 2: 1-(1-((2-(trimethylsilyl)ethoxy)methyl)-1H-imidazol-2-yl)ethanol

To a solution of the product from the previous step (1.00 g, 4.42 mmol) in THF (15 mL) at 0° C. was added MeMgBr in Et₂O (3.0 M, 2.21 mL, 6.63 mmol). The mixture was stirred for 1 h at 0° C., then allowed to warm to RT and stirred overnight. The mixture was treated with sat. aq. NH₄Cl, then extracted with EtOAc (3×30 mL). The combined organic layers were washed with sat. aq. NaCl (30 mL), dried over Na₂SO₄, filtered and concentrated under reduced pressure to give the title compound as a yellow oil, which was used without further purification (1.04 g, 97%).

MS (ES⁺) C₁₁H₂₂N₂O₂Si requires: 242, found: 243 [M+H]⁺.

### Step 3: 1-(1H-imidazol-2-yl)ethanol

To a solution of the product from the previous step (1.12 g, 4.62 mmol) in CH₂Cl₂ (5 mL) was added TFA (5 mL). The mixture was stirred at RT overnight, then concentrated to obtain the title compound as a yellow oil, which was used without further purification (0.510 g, 98%).

MS (ES⁺) C₅H₈N₂O requires: 112, found: 113 [M+H]⁺.

### Step 4: 8-methyl-6,8-dihydro-5H-imidazo[2,1-c][1,4]oxazine

To a mixture of the product from the previous step (0.500 g, 4.46 mmol), K₂CO₃ (1.24 g, 8.92 mmol) and benzyltriethylammonium chloride (0.103 g, 446 mmol) in acetone (10 mL) was added 1,2-dibromoethane (0.770 mL, 8.92 mmol). The mixture was stirred at reflux overnight, then filtered and concentrated under reduced pressure. The residue was purified by SiO₂ gel chromatography (0% to 5% MeOH in CH₂Cl₂) to give the title compound as a yellow solid (0.217 g, 35%).

MS (ES⁺) C₇H₁₀N₂O requires: 138, found: 139 [M+H]⁺.

### Step 5: 2,3-diiodo-8-methyl-6,8-dihydro-5H-imidazo[2,1-c][1,4]oxazine

To a solution of the product from the previous step (217 mg, 1.57 mmol) in DMF (5 mL) was added NIS (954 mg, 4.24 mmol). The mixture was stirred at 60° C. overnight, then poured into water and extracted with EtOAc (3×30 mL). The combined organic layers were washed with sat. aq. NaCl (4×30 mL), dried over Na₂SO₄, and concentrated under reduced pressure. The residue was purified by SiO₂ gel chromatography (0% to 25% EtOAc in CH₂Cl₂) to give the title compound as a yellow solid (100.0 mg, 16%).

MS (ES⁺) C₇H₈I₂N₂O requires: 390, found: 391 [M+H]⁺.

### Step 6: 2-iodo-8-methyl-6, 8-dihydro-5H-imidazo[2,1-c][1,4]oxazine

To a solution of the product from the previous step (100 mg, 256 mmol) in THF (5 mL) at −20° C. was added EtMgBr in Et₂O (3.0 M, 0.128 mL, 0.384 mmol). The mixture was stirred at −20° C. for 30 min, then treated with sat. aq. NH₄Cl and extracted with CH₂Cl₂ (2×15 mL). The combined organic layers were washed with sat. aq. NaCl (10 mL), dried over Na₂SO₄, and concentrated under reduced pressure to give the title compound as a yellow solid.

MS (ES⁺) C₇H₉IN₂O requires: 264, found: 265 [M+H]⁺.

### Step 7: 5-(8-methyl-6,8-dihydro-5H-imidazo[2,1-c][1,4]oxazin-2-yl)-3-(trifluoromethyl)pyridin-2-amine

A mixture of the product from the previous step (50.0 mg, 189 mmol), 5-(4,4,5,5-tetramethyl-1,3,2-dioxaborolan-2-yl)-3-(trifluoromethyl) pyridin-2-amine (65.5 mg, 227 mmol), Cs₂CO₃ (185.1 mg, 568.0 mmol) and Pd(dppf)Cl₂ (15.8 mg, 18.9 mmol) in 5:1 dioxane:water (5 mL) was degassed and purged with N₂. The mixture was heated at 90° C. overnight, then concentrated under reduced pressure. The residue was purified by SiO₂ gel chromatography (0% to 5% MeOH in CH₂Cl₂) to give the title compound as a light brown solid (8.0 mg, 14%).

MS (ES⁺) C₁₃H₁₃F₃N₄O requires: 298, found: 299 [M+H]⁺.

¹H NMR (500 MHz, CDCl₃) δ 8.55 (appar s, 1H), 8.10 (appar s, 1H), 7.07 (s, 1H), 4.96 (s, 2H), 4.89 (q, J=10.0 Hz, 1H), 4.28-4.15 (m, 2H), 4.00-3.94 (m, 2H), 1.69-1.67 (d, J=9.6 Hz, 3H).

### Example 11

### 5-(1-((1R,5S,6s)-3-azabicyclo[3.1.0]hexan-6-yl)-2-isopropyl-1H-imidazol-4-yl)-3-(trifluoromethyl)pyridin-2-amine

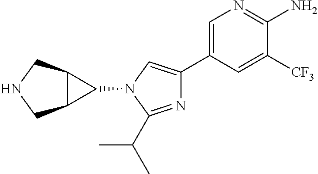

### Step 1: (1R,5S,6s)-tert-butyl 6-(2-isopropyl-1H-imidazol-1-yl)-3-azabicyclo[3.1.0]hexane-3-carboxylate

To a solution of aqueous glyoxal (585.8 mg, 10.1 mmol) and isobutyraldehyde (1.45 g, 20.2 mmol) in MeOH (10.0 mL) were successively added a solution of (1R,5S,6s)-tert-butyl 6-amino-3-azabicyclo[3.1.0]hexane-3-carboxylate (2.00 g, 10.1 mmol) in MeOH (5.0 mL) and a solution of ammonium acetate (777.7 mg, 10.10 mmol) in MeOH (5.0 mL). The mixture was stirred at RT for 18 h, then concentrated under reduced pressure. The residue was purified by SiO₂ gel chromatography (2:1 petroleum ether/EtOAc) to give the title compound as a colorless oil (1.57 g, 53%).

MS (ES⁺) C₁₆H₂₅N₃O₂ requires: 291, found: 292 [M+H]⁺.

### Step 2: (1R,5S,6s)-tert-butyl 6-(4,5-diiodo-2-isopropyl-1H-imidazol-1-yl)-3-azabicyclo[3.1.0]hexane-3-carboxylate

To a solution of the product from the previous step (1.57 g, 5.39 mmol) in DMF (5 mL) was added N-iodosuccinimide (3.02 g, 13.5 mmol), and the mixture was stirred at 70° C. for 3 h. The mixture was then treated with water, extracted with EtOAc (45 mL×3), and the combined organic layers were washed with sat. aq. NaCl (30 mL×6), dried over Na₂SO₄, filtered and concentrated under reduced pressure. The residue was purified by SiO₂ gel chromatography (4:1 petroleum ether/EtOAc) to give the title compound as a yellow oil (1.00 g, 34%).

MS (ES⁺) C₁₆H₂₃I₂N₃O₂ requires: 543, found: 544 [M+H]⁺.

### Step 3: (1R,5S,6s)-tert-butyl 6-(4-iodo-2-isopropyl-1H-imidazol-1-yl)-3-azabicyclo[3.1.0]hexane-3-carboxylate

To a solution of the product from the previous step (1.00 g, 1.84 mmol) in THF (10.0 mL) at −78° C. was added a solution of ethylmagnesium bromide in THF (2.0 M, 1.84 mL, 3.68 mmol). The reaction mixture was stirred for 30 min, then allowed to warm to RT and stirred at RT for 30 min. The mixture was treated with sat. aq. NH₄Cl and extracted with EtOAc (30 mL×3). The combined organic layers were washed with sat. aq. NaCl (30 mL), dried over Na₂SO₄, filtered and concentrated under reduced pressure to give the crude title compound as a colorless oil (380 mg, 49%), which was used without further purification.

MS (ES⁺) C₁₆H₂₄IN₃O₂ requires: 417, found: 418 [M+H]⁺.

### Step 4: (1R,5S,6s)-tert-butyl 6-(4-(6-amino-5-(trifluoromethyl)pyridin-3-yl)-2-isopropyl-1H-imidazol-1-yl)-3-azabicyclo[3.1.0]hexane-3-carboxylate

A mixture of the product from the previous step (120 mg, 0.288 mmol), 5-(4,4,5,5-tetramethyl-1,3,2-dioxaborolan-2-yl)-3-(trifluoromethyl)pyridin-2-amine (99.4 mg, 0.345 mmol), Cs₂CO₃ (281 mg; 0.863 mmol) and (1,1′-bis(diphenylphosphino) ferrocene)palladium(II) chloride (23.5 mg; 0.029 mmol) in 5:1 1,4-dioxane/water (5 mL) was degassed and purged with N₂, then stirred at 80° C. overnight. The mixture was concentrated under reduced pressure, and the residue was purified by SiO₂ gel chromatography (0% to 5% MeOH in DCM) to give the title compound as a colorless oil (100 mg, 77%).

MS (ES⁺) C₂₂H₂₈F₃N₅O₂ requires: 451, found: 452 [M+H]⁺.

### Step 5: 5-(1-((1R,5S,6s)-3-azabicyclo[3.1.0]hexan-6-yl)-2-isopropyl-1H-imidazol-4-yl)-3-(trifluoromethyl)pyridin-2-amine

To a solution of the product from the previous step (10 mg, 22 μmol) in DCM (2 mL) was added TFA (2 mL), and the mixture was stirred at RT for 4 h then concentrated under reduced pressure to give the crude title compound. In analogous experiments for which material was carried forward to subsequent reactions, this compound was used without further purification. For this particular experiment, the residue was purified by reverse phase preparative HPLC (Mobile phase: A=10 mM ammonium bicarbonate/water, B=acetonitrile; Gradient: B=60%-95% in 18 min; Column: C18) to give the title compound (1.5 mg, 19%).

MS (ES⁺) C₁₇H₂₀F₃N₅ requires: 351, found: 352 [M+H]⁺.

¹H NMR (400 MHz, MeOD) δ 8.50 (d, J=1.5 Hz, 1H), 8.13 (d, J=1.7 Hz, 1H), 7.34 (s, 1H), 3.40-3.25 (m, 3H), 3.17 (appar s, 1H), 2.99 (appar d, J=11.8 Hz, 2H), 2.16 (appar s, 2H), 1.36 (d, J=14.1 Hz, 6H).

### Example 12

### 5-(2-isopropyl-1-((1R,5S,6s)-3-(oxetan-3-yl)-3-azabicyclo[3.1.0]hexan-6-yl)-1H-imidazol-4-yl)-3-(trifluoromethyl)pyridin-2-amine

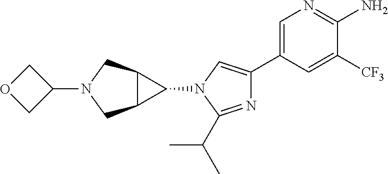 [SMILES: CC(C)c1nc(-c2cnc(N)c(C(F)(F)F)c2)cn1[C@H]1[C@@H]2CN(C3COC3)C[C@@H]21]

To a solution of the Example 11 compound (15 mg, 0.043 mmol) and oxetan-3-one (5.0 mg, 0.065 mmol) in DCM (1 mL) was added sodium cyanoborohydride (3.0 mg, 0.052 mmol). The resulting mixture was stirred at RT for 16 h, then treated with sat. aq. NH₄Cl and extracted with EtOAc (3×50 mL). The combined organic layers were dried over Na₂SO₄, filtered and concentrated under reduced pressure. The residue was purified by reverse phase preparative HPLC (Mobile phase: A=10 mM ammonium bicarbonate/water, B=acetonitrile; Gradient: B=60%-95% in 18 min; Column: C18) to give the title compound (4 mg, 23%).

MS (ES⁺) C₂₀H₂₄F₃N₅O requires: 407, found: 408 [M+H]⁺.

¹H NMR (500 MHz, CDCl₃): δ 8.52 (d, J=1.4 Hz, 1H), 8.09 (d, J=1.8 Hz, 1H), 6.95 (s, 1H), 4.91 (s, 2H), 4.70 (t, J=6.7 Hz, 2H), 4.62 (t, J=6.1 Hz, 2H), 3.79 (appar quin, J=6.5 Hz, 1H), 3.56 (s, 1H), 3.28-3.18 (m, 3H), 2.54 (d, J=8.6 Hz, 2H), 2.00 (s, 2H), 1.39 (d, J=6.9 Hz, 6H).

### Example 13

### 5-(2-isopropyl-1-((1R,5S,6s)-3-(tetrahydro-2H-pyran-4-yl)-3-azabicyclo[3.1.0]hexan-6-yl)-1H-imidazol-4-yl)-3-(trifluoromethyl)pyridin-2-amine

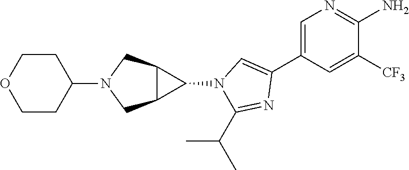 [SMILES: CC(C)c1nc(-c2cnc(N)c(C(F)(F)F)c2)cn1[C@H]1[C@@H]2CN(C3CCOCC3)C[C@@H]21]

To a solution of the Example 11 compound (40 mg, 0.11 mmol) and dihydro-2H-pyran-4(3H)-one (17 mg, 0.17 mmol) in MeOH (1 mL) was added sodium cyanoborohydride (8.0 mg, 0.13 mmol). The resulting mixture was stirred at RT for 16 h, then treated with sat. aq. NH₄Cl and extracted with EtOAc (3×50 mL). The combined organic layer were dried over Na₂SO₄, filtered and concentrated under reduced pressure. The residue was purified by reverse phase preparative HPLC (Mobile phase: A=10 mM ammonium bicarbonate/water, B=acetonitrile; Gradient: B=60%-95% in 18 min; Column: C18) to give the title compound (5 mg, 10%).

MS (ES⁺) C₂₂H₂₈F₃N₅O requires: 435, found: 436 [M+H]⁺.

¹H NMR (400 MHz, CDCl₃) δ 8.52 (appar s, 1H), 8.09 (appar s, 1H), 6.94 (s, 1H), 4.90 (s, 2H), 3.97 (appar d, J=11.4 Hz, 2H), 3.47 (s, 1H), 3.40 (dd, J=11.5, 9.6 Hz, 2H), 3.30 (d, J=8.9 Hz, 2H), 3.25-3.20 (m, 1H), 2.52 (appar d, J=8.4 Hz, 2H), 2.33 (appar t, J=10.4 Hz, 1H), 1.99 (appar s, 2H), 1.76-1.70 (m, 2H), 1.62-1.45 (m, 2H), 1.38 (d, J=6.9 Hz, 6H).

### Example 14

### 1-(6-(4-(6-amino-5-(trifluoromethyl)pyridin-3-yl)-2-isopropyl-1H-imidazol-1-yl)-3-azabicyclo[3.1.0]hexan-3-yl)ethanone

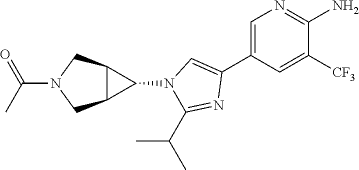 [SMILES: CC(=O)N1C[C@@H]2[C@H](C1)[C@H]2n1cc(-c2cnc(N)c(C(F)(F)F)c2)nc1C(C)C]

To a solution of the Example 11 compound (15 mg, 0.043 mmol) in DCM (1 mL) was added acetyl chloride (4.0 mg, 0.043 mmol). The resulting mixture was stirred at RT for 30 min, then concentrated under reduced pressure. The residue was purified by reverse phase preparative HPLC (Mobile phase: A=10 mM ammonium bicarbonate/water, B=acetonitrile; Gradient: B=60%-95% in 18 min; Column: C18) to give the title compound (2 mg, 12%).

MS (ES⁺) C₁₉H₂₂F₃N₅O requires: 393, found: 394 [M+H]⁺.

¹H NMR (500 MHz, CDCl₃): δ 8.52 (d, J=1.5 Hz, 1H), 8.09 (d, J=1.8 Hz, 1H), 6.95 (s, 1H), 4.94 (s, 2H), 4.06 (d, J=12.3 Hz, 1H), 3.79 (dt, J=10.5, 7.3 Hz, 2H), 3.60 (dd, J=12.3, 4.7 Hz, 1H), 3.17 (appar quin, J=6.8 Hz, 1H), 2.99 (t, J=2.3 Hz, 1H), 2.28-2.15 (m, 2H), 2.07 (s, 3H), 1.38 (t, J=7.0 Hz, 6H).

### Example 15

### 5-(2-isopropyl-1-((1R,5S,6s)-3-(2-methoxyethyl)-3-azabicyclo[3.1.0]hexan-6-yl)-1H-imidazol-4-yl)-3-(trifluoromethyl)pyridin-2-amine

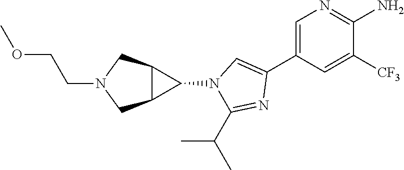 [SMILES: COCCN1C[C@@H]2[C@H](C1)[C@H]2n1cc(-c2cnc(N)c(C(F)(F)F)c2)nc1C(C)C]

To a solution of the Example 11 compound (20 mg, 0.057 mmol) and 1-bromo-2-methoxyethane (12 mg, 0.086 mmol) in DMF (1 mL) was added DIEA (11 mg, 0.086 mmol). The resulting mixture was stirred at 50° C. for 16 h, then concentrated under reduced pressure. The residue was purified by reverse phase preparative HPLC (Mobile phase: A=10 mM ammonium bicarbonate/water, B=acetonitrile; Gradient: B=60%-95% in 18 min; Column: C18) to give the title compound (5 mg, 21%).

MS (ES⁺) C₂₀H₂₆F₃N₅O requires: 409, found: 410 [M+H]⁺.

¹H NMR (500 MHz, CDCl₃) δ 8.52 (d, J=1.4 Hz, 1H), 8.08 (d, J=1.8 Hz, 1H), 6.94 (s, 1H), 4.90 (s, 2H), 3.52 (appar s, 1H), 3.48 (t, J=5.7 Hz, 2H), 3.37 (s, 3H), 3.31 (d, J=9.0 Hz, 2H), 3.23 (appar quin, J=6.9 Hz, 1H), 2.69 (t, J=5.7 Hz, 2H), 2.54 (d, J=8.9 Hz, 2H), 1.94 (s, 2H), 1.37 (d, J=6.9 Hz, 6H).

### Example 16

### 5-(1-((1R,5S,6s)-3-(2-fluoroethyl)-3-azabicyclo[3.1.0]hexan-6-yl)-2-isopropyl-1H-imidazol-4-yl)-3-(trifluoromethyl)pyridin-2-amine

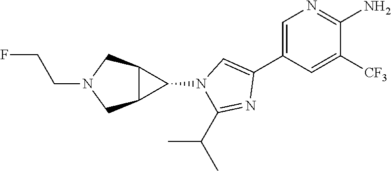 [SMILES: CC(C)c1nc(-c2cnc(N)c(C(F)(F)F)c2)cn1[C@H]1[C@@H]2CN(CCF)C[C@@H]21]

To a solution of the Example 11 compound (30 mg, 0.09 mmol) and 1-bromo-2-fluoroethane (14 mg, 0.11 mmol) in DMF (1 mL) was added DIEA (6 mg, 0.05 mmol). The mixture was stirred at 50° C. for 16 h, then concentrated under reduced pressure. The residue was purified by reverse phase preparative HPLC (Mobile phase: A=10 mM NH₄HCO₃/H₂O, B=acetonitrile; Gradient: B=60%-95% in 18 min; Column: C18) to give the title compound (9.5 mg, 27%).

MS (ES⁺) C₁₉H₂₃F₄N₅ requires: 397, found: 398 [M+H]⁺.

¹H NMR (400 MHz, CDCl₃) δ 8.52 (appar s, 1H), 8.09 (appar s, 1H), 6.94 (s, 1H), 4.89 (s, 2H), 4.64-4.53 (m, 1H), 4.51-4.41 (m, 1H), 3.52 (s, 1H), 3.33 (d, J=8.9 Hz, 2H), 3.23 (appar quin, J=6.7 Hz, 1H), 2.91-2.68 (m, 2H), 2.60 (d, J=8.7 Hz, 2H), 1.96 (appar s, 2H), 1.38 (d, J=6.9 Hz, 6H).

### Example 17

### 5-(1-((1R,5S,6s)-3-(2,2-difluoroethyl)-3-azabicyclo[3.1.0]hexan-6-yl)-2-isopropyl-1H-imidazol-4-yl)-3-(trifluoromethyl)pyridin-2-amine

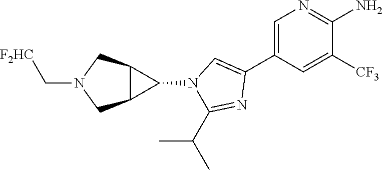 [SMILES: CC(C)c1nc(-c2cnc(N)c(C(F)(F)F)c2)cn1[C@H]1[C@@H]2CN(CC(F)F)C[C@@H]21]

To a solution of the Example 11 compound (30 mg, 0.085 mmol) and 2,2-difluoroethyl trifluoromethanesulfonate (27.0 mg, 0.128 mmol) in THF (1 mL) was added DIEA (16.0 mg, 0.128 mmol). The resulting mixture was stirred at reflux for 16 h, then concentrated under reduced pressure. The residue was purified by reverse phase preparative HPLC (Mobile phase: A=10 mM ammonium bicarbonate/water, B=acetonitrile; Gradient: B=60%-95% in 18 min; Column: C18) to give the title compound (5 mg, 14%).

MS (ES⁺) C₁₉H₂₂F₅N₅ requires: 415, found: 416 [M+H]⁺.

¹H NMR (400 MHz, CDCl₃): δ 8.51 (appar s, 1H), 8.08 (d, J=1.7 Hz, 1H), 6.94 (s, 1H), 5.82 (tt, J=55.9, 4.3 Hz, 1H), 4.90 (s, 2H), 3.47 (s, 1H), 3.34 (d, J=9.0 Hz, 2H), 3.21 (appar quin, J=6.9 Hz, 1H), 2.88 (td, J=15.1, 4.3 Hz, 2H), 2.69 (appar d, J=8.9 Hz, 2H), 1.98 (appar s, 2H), 1.41-1.25 (d, J=6.9 Hz, 6H).

### Example 18

### 5-(2-isopropyl-1-((1R,5S,6s)-3-(2,2,2-trifluoroethyl)-3-azabicyclo[3.1.0]hexan-6-yl)-1H-imidazol-4-yl)-3-(trifluoromethyl)pyridin-2-amine

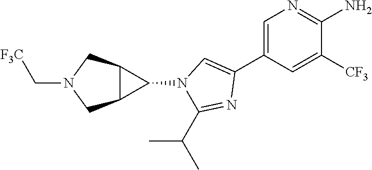 [SMILES: CC(C)c1nc(-c2cnc(N)c(C(F)(F)F)c2)cn1[C@H]1[C@@H]2CN(CC(F)(F)F)C[C@@H]21]

To a solution of the Example 11 compound (15 mg, 0.043 mmol) and 2,2,2-trifluoroethyl trifluoromethanesulfonate (15 mg, 0.065 mmol) in THF (1 mL) was added DIEA (8.0 mg, 0.065 mmol). The resulting mixture was stirred at reflux for 16 h, then concentrated under reduced pressure. The residue was purified by reverse phase preparative HPLC (Mobile phase: A=10 mM ammonium bicarbonate/water, B=acetonitrile; Gradient: B=60%-95% in 18 min; Column: C18) to give the title compound (5 mg, 27%). MS (ES⁺) C₁₉H₂₁F₆N₅ requires: 433, found: 434 [M+H]⁺. ¹H NMR (500 MHz, CDCl₃): δ 8.52 (appar s, 1H), 8.09 (appar s, 1H), 6.94 (s, 1H), 4.90 (s, 2H), 3.47 (s, 1H), 3.38 (d, J=8.8 Hz, 2H), 3.21 (appar quin, J=6.8 Hz, 1H), 3.12 (q, J=9.4 Hz, 2H), 2.83 (appar d, J=8.7 Hz, 2H), 2.00 (appar s, 2H), 1.38 (d, J=6.9 Hz, 6H).

### Examples 19a and 19b

### 5-(2-isopropyl-1-((1R,5S,6r)-3-morpholinobicyclo[3.1.0]hexan-6-yl)-1H-imidazol-4-yl)-3-(trifluoromethoxy)pyridin-2-amine and 5-(2-isopropyl-1-((1R,5S,6s)-3-morpholinobicyclo[3.1.0]hexan-6-yl)-1H-imidazol-4-yl)-3-(trifluoromethoxy)pyridin-2-amine

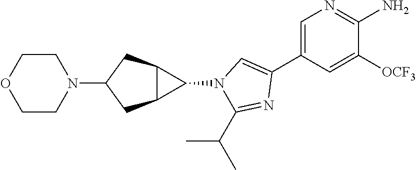

### Step 1: (1R,5S,6r)-6-(4-(6-amino-5-(trifluoromethoxy)pyridin-3-yl)-2-isopropyl-1H-imidazol-1-yl)bicyclo[3.1.0]hexan-3-one

A mixture of (1R,5S,6r)-6-(4-iodo-2-isopropyl-1H-imidazol-1-yl)bicyclo[3.1.0]hexan-3-one (2.95 g, 8.93 mmol), 5-(4,4,5,5-tetramethyl-1,3,2-dioxaborolan-2-yl)-3-(trifluoromethoxy)pyridin-2-amine (3.53 g, 11.6 mmol), K₂CO₃ in water (2.0 M, 22.34 mL, 44.68 mmol) and (1,1′-bis(diphenylphosphino)ferrocene)palladium(II) chloride (1.12 g, 1.34 mmol) in DMF (50 mL) was degassed and purged with N₂, stirred at 90° C. for 30 min., allowed to cool then concentrated under reduced pressure. The residue was purified by SiO₂ gel chromatography (10% to 80% EtOAc in petroleum ether) to give the title compound as a yellow solid (2.77 g, 82%). MS (ES⁺) C₁₈H₁₉F₃N₄O₂ requires: 380, found: 381 [M+H]⁺.

### Step 2: 5-(2-isopropyl-1-((1R,5S,6r)-3-morpholinobicyclo[3.1.0]hexan-6-yl)-1H-imidazol-4-yl)-3-(trifluoromethoxy)pyridin-2-amine and 5-(2-isopropyl-1-((1R,5S,6s)-3-morpholinobicyclo[3.1.0]hexan-6-yl)-1H-imidazol-4-yl)-3-(trifluoromethoxy)pyridin-2-amine

To a solution of the product from the previous step (2.77 g, 7.28 mmol) and morpholine (951.7 mg, 10.92 mmol) in MeOH (150 mL) was added sodium cyanoborohydride (1.37 g, 21.8 mmol). The resulting mixture was stirred at RT for 2 d, then concentrated under reduced pressure. The residue was purified by reverse phase preparative HPLC (Mobile phase: A=10 mM ammonium bicarbonate/water, B=acetonitrile; Gradient: B=5%-95% in 18 min; Column: C18) to give the title compounds as two separated isomers.

Example 19a: white solid (759 mg, 23%); retention time=1.86 min.

MS (ES⁺) C₂₂H₂₈F₃N₅O₂ requires: 451, found: 452 [M+H]⁺.

¹H NMR (400 MHz, CDCl₃) δ 8.30 (d, J=1.8 Hz, 1H), 7.77 (appar s, 1H), 6.91 (s, 1H), 4.65 (s, 2H), 3.82-3.59 (m, 4H), 3.23-3.13 (m, 1H), 2.92 (appar s, 1H), 2.45 (appar br s, 4H), 2.35-2.21 (m, 3H), 1.93-1.83 (m, 4H), 1.37 (d, J=6.9 Hz, 6H).

Example 19b: white solid (908 mg, 28%); retention time=1.95 min.

MS (ES⁺) C₂₂H₂₈F₃N₅O₂ requires: 451, found: 452 [M+H]⁺.

¹H NMR (400 MHz, CDCl₃) δ 8.30 (appar s, 1H), 7.77 (appar s, 1H), 6.91 (s, 1H), 4.65 (s, 2H), 3.70 (appar br s, 4H), 3.31-3.09 (m, 2H), 2.95-2.79 (m, 1H), 2.44 (appar br s, 4H), 2.35-2.21 (m, 2H), 1.84 (appar s, 2H), 1.78-1.68 (m, 2H), 1.37 (d, J=6.8 Hz, 6H).

### Examples 20a and 20b

### 5-(1-((1R,5S,6r)-3-(1,4-oxazepan-4-yl)bicyclo[3.1.0]hexan-6-yl)-2-isopropyl-1H-imidazol-4-yl)-3-(trifluoromethoxy)pyridin-2-amine and 5-(1-((1R,5S,6s)-3-(1,4-oxazepan-4-yl)bicyclo[3.1.0]hexan-6-yl)-2-isopropyl-1H-imidazol-4-yl)-3-(trifluoromethoxy)pyridin-2-amine

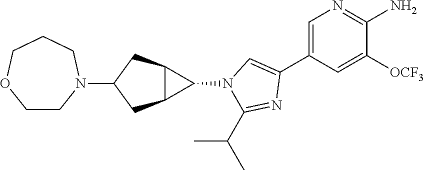

### Step 1: 4-(6-(4-iodo-2-isopropyl-1H-imidazol-1-yl)bicyclo[3.1.0]hexan-3-yl)-1,4-oxazepane

To a solution of 6-(4-iodo-2-isopropyl-1H-imidazol-1-yl)bicyclo[3.1.0]hexan-3-one (200 mg, 0.606 mmol) and 1,4-oxazepane hydrochloride (175 mg, 1.21 mmol) in methanol (8 mL) was added sodium cyanoborohydride (190 mg, 3.03 mmol). The resulting mixture was stirred at RT overnight, then concentrated. The residue was purified by SiO₂ gel chromatography (0% to 5% MeOH in DCM) to give the title compound as a yellow solid (211 mg, 84%). MS (ES⁺) C₁₇H₂₆IN₃O requires: 415, found: 416 [M+H]⁺.

### Step 2: 5-(1-((1R,5S,6r)-3-(1,4-oxazepan-4-yl)bicyclo[3.1.0]hexan-6-yl)-2-isopropyl-1H-imidazol-4-yl)-3-(trifluoromethoxy)pyridin-2-amine and 5-(1-((1R,5S,6s)-3-(1,4-oxazepan-4-yl)bicyclo[3.1.0]hexan-6-yl)-2-isopropyl-1H-imidazol-4-yl)-3-(trifluoromethoxy)pyridin-2-amine

A mixture of 4-(6-(4-iodo-2-isopropyl-1H-imidazol-1-yl)bicyclo[3.1.0]hexan-3-yl)-1,4-oxazepane (211 mg, 0.508 mmol), 5-(4,4,5,5-tetramethyl-1,3,2-dioxaborolan-2-yl)-3-(trifluoromethoxy)pyridin-2-amine (309 mg, 1.02 mmol), aqueous K₂CO₃ (2.0 M, 1.27 mL, 2.54 mmol) and (1,1′-bis(diphenylphosphino) ferrocene)palladium(II) chloride (63.5 mg, 0.076 mmol) in DMF (3 mL) was degassed and purged with N₂, then stirred at 90° C. for 30 min. The mixture was allowed to cool then filtered, and the filtrate was purified by reverse phase preparative HPLC (Mobile phase: A=10 mM ammonium bicarbonate/water, B=acetonitrile; Gradient: B=5%-95% in 18 min; Column: C18) to give the title compounds as two separated isomers.

Example 20a: white solid (19.6 mg, 8%); retention time=1.82 min.

MS (ES⁺) C₂₃H₃₀F₃N₅O₂ requires: 465, found: 466 [M+H]⁺.

¹H NMR (500 MHz, CDCl₃) δ 8.30 (d, J=1.7 Hz, 1H), 7.77 (appar s, 1H), 6.91 (s, 1H), 4.66 (s, 2H), 3.80 (t, J=6.0 Hz, 2H), 3.77-3.67 (m, 2H), 3.24-3.11 (m, 1H), 2.92 (appar s, 1H), 2.80-2.68 (m, 5H), 2.32-2.22 (m, 2H), 1.94-1.87 (m, 6H), 1.36 (d, J=6.9 Hz, 6H).

Example 20b: white solid; retention time=1.87 min.

MS (ES⁺) C₂₃H₃₀F₃N₅O₂ requires: 465, found: 466 [M+H]⁺.

¹H NMR (500 MHz, CDCl₃) δ 8.29 (d, J=1.7 Hz, 1H), 7.77 (appar s, 1H), 6.89 (s, 1H), 4.69 (s, 2H), 3.80 (t, J=5.9 Hz, 4H), 3.58-3.35 (m, 1H), 3.33-3.12 (m, 2H), 3.00-2.63 (m, 3H), 2.56-2.33 (m, 2H), 2.25-1.76 (m, 7H), 1.37 (d, J=6.9 Hz, 6H).

### Example 21

### 5-(2-isopropyl-1-methyl-1H-imidazol-4-yl)-3-(trifluoromethyl)pyridin-2-amine

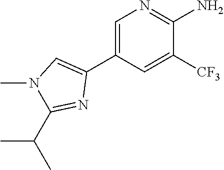 [SMILES: CC(C)c1nc(-c2cnc(N)c(C(F)(F)F)c2)cn1C]

### Step 1: 2-isopropyl-1-methyl-1H-imidazole

To a solution of 2-isopropyl-1H-imidazole (1.1 g, 10 mmol) in DMF (15 mL) at 0° C. was added NaH (0.48 g, 20 mmol), then iodomethane (2.8 g, 20 mmol). The mixture was stirred at 0° C. for 1 h, then allowed to warm to RT and stirred at RT for 3 h. The mixture was treated with sat. aq. NH₄Cl then extracted with EtOAc (30 mL×3). The combined organic layers were washed with sat. aq. NaCl (30 mL), dried over Na₂SO₄, filtered and concentrated under reduced pressure to give the crude title compound as a yellow oil (1.2 g, 96%), which was used without further purification.

MS (ES⁺) C₇H₁₂N₂ requires: 124, found: 125 [M+H]⁺.

### Step 2: 4,5-diiodo-2-isopropyl-1-methyl-1H-imidazole

To a solution of the product from the previous step (500 mg, 4 mmol) in THF (10 mL) was added N-iodosuccinimide (2.2 g, 10 mmol), and the mixture was stirred at RT for 2 h. The mixture was treated with sat. aq. sodium thiosulfate and extracted with EtOAc (30 mL×3). The combined organic layers were washed with sat. aq. NaCl (30 mL), dried over Na₂SO₄, filtered and concentrated under reduced pressure to give the crude title compound as a white solid (300 mg, 20%), which was used without further purification.

MS (ES⁺) C₇H₁₀I₂N₂ requires: 376, found: 377 [M+H]⁺.

### Step 3: 4-iodo-2-isopropyl-1-methyl-1H-imidazole

To a solution of the product from the previous step (300 mg, 0.79 mmol) in THF (8 mL) at −78° C. was added dropwise a solution of ethylmagnesium bromide in THF (2.5 M, 0.64 mL, 1.6 mmol). The resulting solution was stirred at −78° C. for 2 h, then treated with ice water and extracted with EtOAc (20 mL×3). The combined organic layers were washed with sat. aq. NaCl (10 mL), dried over Na₂SO₄, filtered and concentrated under reduced pressure to give the crude title compound as a yellow solid (120 mg, 60%), which was used without further purification.

MS (ES⁺) C₇H₁₁IN₂ requires: 250, found: 251 [M+H]⁺.

### Step 4: 5-(2-isopropyl-1-methyl-1H-imidazol-4-yl)-3-(trifluoromethyl)pyridin-2-amine

To a mixture of the product from the previous step (120 mg, 0.48 mmol) in 1,4-dioxane (3 mL) were added 5-(4,4,5,5-tetramethyl-1,3,2-dioxaborolan-2-yl)-3-(trifluoromethyl)pyridin-2-amine (207 mg, 0.72 mmol), (1,1′-bis(diphenylphosphino) ferrocene)palladium(II) chloride (39 mg, 0.048 mmol), Cs₂CO₃ (312 mg, 0.96 mmol), and water (0.5 mL). The mixture was stirred at 90° C. under N₂ for 3 h, then purified by reverse phase preparative HPLC (Mobile phase: A=10 mM ammonium bicarbonate/water, B=acetonitrile; Gradient: B=60%-95% in 18 min; Column: C18) to give the title compound as a white solid (31 mg, 22%).

MS (ES⁺) C₁₃H₁₅F₃N₄ requires: 284, found: 285 [M+H]⁺.

¹H NMR (400 MHz, DMSO) δ 8.53 (appar s, 1H), 7.96 (appar s, 1H), 7.44 (s, 1H), 6.32 (s, 2H), 3.59 (s, 3H), 3.08-3.05 (m, 1H), 1.24 (d, J=6.9 Hz, 6H).

### Example 22

### 1-(4-(6-amino-5-(trifluoromethyl)pyridin-3-yl)-1-cyclobutyl-1H-imidazol-2-yl)propan-1-ol

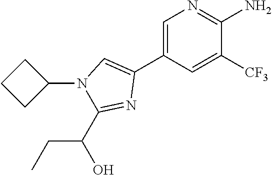 [SMILES: CCC(O)c1nc(-c2cnc(N)c(C(F)(F)F)c2)cn1C1CCC1]

### Step 1: 2-((benzyloxy)methyl)-1-cyclobutyl-1H-imidazole

To a solution of 2-(benzyloxy)acetaldehyde (591 mg, 3.94 mmol) in MeOH (500 ml) at RT was added dropwise cyclobutanamine (280 mg, 3.94 mmol) then ammonium acetate (303 mg, 3.94 mmol). To the mixture was then added dropwise glyoxal (571 mg, 3.94 mmol) and the reaction was stirred at RT for 24 h. Volatiles were removed under reduced pressure, and the remaining mixture was treated with H₂O (500 mL) and sat. aq. NaHCO₃ and extracted with EtOAc (3×200 mL). The combined organic layers were washed with sat. aq. NaCl, dried over MgSO₄, filtered and concentrated under reduced pressure to give the crude title compound as a yellow foam (322 mg, 34%), which was used without further purification.

MS (ES⁺) C₁₅H₁₈N₂O requires: 242, found: 243 [M+H]⁺.

### Step 2: 2-((benzyloxy)methyl)-1-cyclobutyl-4-iodo-1H-imidazole

To a solution of the product from the previous step (320 mg, 1.32 mmol) in DMF (2 ml) was added N-iodosuccinimide (891 mg, 3.96 mmol) and the resulting mixture was stirred at 90° C. for 2 h. To the mixture were added sat. aq. Na₂S₂O₃ (1 ml) and water (10 ml). The mixture was extracted with EtOAc (3×5 mL), and the combined organic layers were washed with sat. aq. NaCl, dried over MgSO₄, filtered and concentrated under reduced pressure. The residue was purified via SiO₂ gel chromatography (0% to 40% EtOAc in hexanes) to give diiodo intermediate (385 mg) as a pale yellow liquid. This liquid was dissolved in THF (2 mL) and the resulting solution was chilled to −78° C., then treated with a solution of isopropylmagnesium chloride in THF (2.0 M, 0.55 mL, 1.1 mmol) and the resulting mixture was stirred at −78° C. for 1 h. To the mixture was added sat. aq. NH₄Cl (10 mL), and the layers were separated. The aqueous phase was extracted with EtOAc (3×5 mL), and the combined organic layers were washed with sat. aq. NaCl, dried over MgSO₄, filtered and concentrated under reduced pressure. The residue was purified via SiO₂ gel chromatography (0% to 50% EtOAc in hexanes) to give the title compound as a white solid (208 mg, 43%).

MS (ES⁺) C₁₅H₁₇IN₂O requires: 368, found: 369 [M+H]⁺.

### Step 3: 5-(2-((benzyloxy)methyl)-1-cyclobutyl-1H-imidazol-4-yl)-3-(trifluoromethyl)pyridin-2-amine

A degassed mixture of the product from the previous step (200 mg, 0.543 mmol) 5-(4,4,5,5-tetramethyl-1,3,2-dioxaborolan-2-yl)-3-(trifluoromethyl)pyridin-2-amine (164 mg, 0.570 mmol), PdCl₂(dppf)-CH₂Cl₂ (22.2 mg, 0.027 mmol) and aqueous K₂CO₃ (2.0 M, 0.543 ml, 1.086 mmol) in DMF (2 ml) was stirred at 90° C. for 1 h. Water (300 ml) and 1 M aq. HCl (50 ml) were added to the mixture, which was then extracted with EtOAc (3×300 ml). The aqueous phase was basified with 10% aq. NaOH to pH 5 and then sat. aq. NaHCO₃ to pH 8, then again extracted with EtOAc (3×200 ml). The combined organic layers were washed with sat. aq. NaCl, dried over MgSO₄, filtered and concentrated under reduced pressure. The residue was purified via SiO₂ gel chromatography (0% to 5% MeOH in DCM) to give the title compound as an off-white solid (205 mg, 94%).

MS (ES⁺) C₂₁H₂₁F₃N₄O requires: 402, found: 403 [M+H]⁺.

### Step 4: (4-(6-amino-5-(trifluoromethyl)pyridin-3-yl)-1-cyclobutyl-1H-imidazol-2-yl)methanol

A solution of the product from the previous step (200 mg, 0.497 mmol) in TFA (3 ml) was stirred at 70° C. for 16 h, then concentrated under reduced pressure. The residue was diluted with water and the pH was adjusted with NaHCO₃ was to pH 8. Solid was isolated by filtration to give the title compound as a white solid (128 mg, 82%).

MS (ES⁺) C₁₄H₁₅F₃N₄O requires: 312, found: 313 [M+H]±.

### Step 5: 4-(6-amino-5-(trifluoromethyl)pyridin-3-yl)-1-cyclobutyl-1H-imidazole-2-carbaldehyde

To a solution of the product from the previous step (128 mg, 0.410 mmol) in DCM (4 ml) was added MnO₂ (178 mg, 2.05 mmol) and the resulting mixture was stirred at 20° C. for 16 h. The reaction mixture was filtered through Celite, and the filtrate was concentrated under reduced pressure. The residue was purified via SiO₂ gel chromatography (0% to 5% MeOH in DCM) to give the title compound as a white solid (96 mg, 75%). MS (ES⁺) C₁₄H₁₃F₃N₄O requires: 310, found: 311 [M+H]⁺.

### Step 6: 1-(4-(6-amino-5-(trifluoromethyl)pyridin-3-yl)-1-cyclobutyl-1H-imidazol-2-yl)propan-1-ol

To a solution of the product from the previous step (45 mg, 0.14 mmol) in THF (1 ml) at 0° C. was added a solution of ethylmagnesium bromide in THF (1.0 M, 0.725 ml, 0.725 mmol) and the resulting mixture was stirred at 20° C. for 6 h. To the mixture was added sat. aq. NH₄Cl (5 mL), and the layers were separated. The aqueous phase was extracted with EtOAc (3×5 mL), and the combined organic layers were washed with sat. aq. NaCl, dried over MgSO₄, filtered and concentrated under reduced pressure. The residue was purified via SiO₂ gel chromatography (0% to 5% MeOH in DCM) to give the title compound as a yellow solid (8.0 mg, 16%).

MS (ES⁺) C₁₆H₁₉F₃N₄O requires: 340, found: 341 [M+H]⁺.

¹H NMR (600 MHz, CDCl₃-d) δ 8.55 (appar s, 1H), 8.35 (appar s, 1H), 7.28 (s, 1H), 5.48 (br s, 2H), 4.80-4.65 (m, 2H), 2.68-2.45 (m, 4H), 2.06-1.75 (m, 4H), 1.03 (t, J=7.60 Hz, 3H).

For compounds which are disclosed as a/b pairs, for example, 19a and 19b, the “a” designation refers to the first-eluting compound, and the “b” designation refers to the last-eluting compound. Such compounds are typically stereoisomers, for example epimers, having (R) or (S) configuration at a stereocenter. Each compound is individually exemplified herein, but the absolute configuration may not yet have been characterized and assigned. Both a and b ((R) and (S)), as well as racemic mixtures thereof, are contemplated within the scope of the invention.

TABLE 1
Synthesized Examples

| Ex | Sch. | | |
|---|---|---|---|
| 1 | IX | 5-(1-(cyclopropylmethyl)-2- ((1R,5S,6r)-3-(oxetan-3-yl)-3- azabicyclo[3.1.0]hexan-6-yl)-1H- imidazol-4-yl)-3-(trifluoro- methyl)pyridin-2-amine | 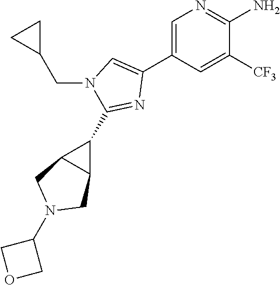 [SMILES: Nc1ncc(-c2cn(CC3CC3)c([C@H]3[C@@H]4CN(C5COC5)C[C@@H]43)n2)cc1C(F)(F)F] |
| 2 | VI | 5-(2-(cyclopropylmethyl)-1- ((1R,5S,6s)-3-(oxetan-3-yl)-3- azabicyclo[3.1.0]hexan-6-yl)-1H- imidazol-4-yl)-3-(trifluoro- methyl)pyridin-2-amine | 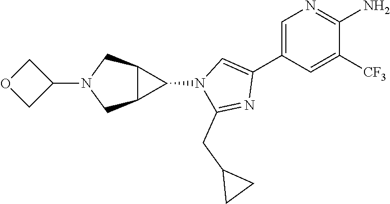 [SMILES: Nc1ncc(-c2cn([C@H]3[C@@H]4CN(C5COC5)C[C@@H]43)c(CC3CC3)n2)cc1C(F)(F)F] |
| 3 | III | 5-(2-isopropyl-1-((1R,5S,6r)-3- morpholinobicyclo[3.1.0]hexan-6- yl)-1H-imidazol-4-yl)-3-(trifluoro- methyl)pyridin-2-amine | 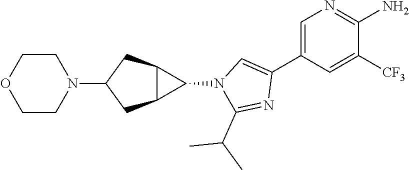 |
| 4 | IV | 5-[8-cyclopropyl-7-(oxetan-3-yl)- 5H,6H,7H,8H-imidazo[1,2- a]pyrazin-2-yl]-3- (trifluoromethyl)pyridin-2-amine | 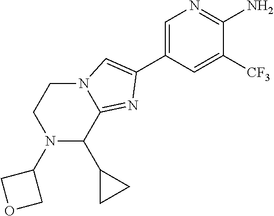 [SMILES: Nc1ncc(-c2cn3c(n2)C(C2CC2)N(C2COC2)CC3)cc1C(F)(F)F] |
| 5 | V | 5-(8-methyl-6,8-dihydro-5H- imidazo[2,1-c][1,4]oxazin-2-yl)-3- (trifluoromethyl)pyridin-2-amine | 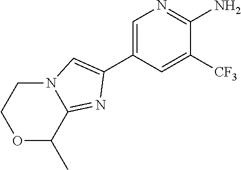 [SMILES: CC1OCCn2cc(-c3cnc(N)c(C(F)(F)F)c3)nc21] |
| 6 | II | 5-[1-cyclopropyl-2-(propan-2-yl)- 1H-imidazol-4-yl]-3-(trifluoro- methyl)pyridin-2-amine | 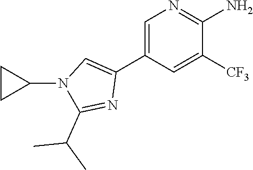 [SMILES: CC(C)c1nc(-c2cnc(N)c(C(F)(F)F)c2)cn1C1CC1] |
| 7 | II | 5-[1-cyclobutyl-2-(propan-2-yl)- 1H-imidazol-4-yl]-3-(trifluoro- methyl)pyridin-2-amine | 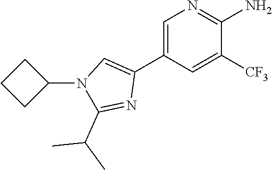 [SMILES: CC(C)c1nc(-c2cnc(N)c(C(F)(F)F)c2)cn1C1CCC1] |
| 8 | II | 5-[2-cyclopropyl-1-(propan-2-yl)- 1H-imidazol-4-yl]-3-(trifluoro- methyl)pyridin-2-amine | 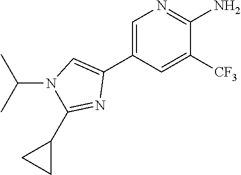 [SMILES: CC(C)n1cc(-c2cnc(N)c(C(F)(F)F)c2)nc1C1CC1] |
| 9 | II | 5-(1,2-dicyclopropyl-1H-imidazol- 4-yl)-3-(trifluoromethyl)pyridin-2- amine | 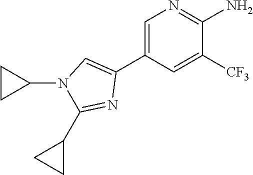 [SMILES: Nc1ncc(-c2cn(C3CC3)c(C3CC3)n2)cc1C(F)(F)F] |
| 10 | V | 5-(5,6-dihydro-8H-imidazo[2,1- c][1,4]oxazin-2-yl)-3-(trifluoro- methyl)pyridin-2-amine | 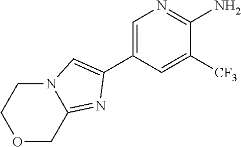 [SMILES: Nc1ncc(-c2cn3c(n2)COCC3)cc1C(F)(F)F] |
| 11 | VI | 5-(1-(3-azabicyclo[3.1.0]hexan-6- yl)-2-isopropyl-1H-imidazol-4-yl)- 3-(trifluoromethyl)pyridin-2- amine | 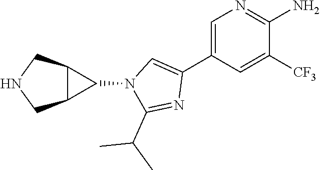 |
| 12 | VI | 5-(2-isopropyl-1-((1R,5S,6s)-3- (oxetan-3-yl)-3-azabicyclo[3.1.0]- hexan-6-yl)-1H-imidazol-4-yl)-3- (trifluoromethyl)pyridin-2-amine | 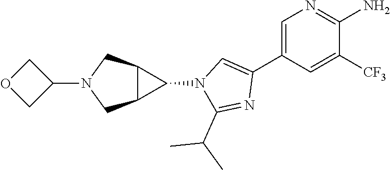 [SMILES: CC(C)c1nc(-c2cnc(N)c(C(F)(F)F)c2)cn1[C@H]1[C@@H]2CN(C3COC3)C[C@@H]21] |
| 13 | VI | 5-(2-isopropyl-1-((1R,5S,6s)-3- (tetrahydro-2H-pyran-4-yl)-3- azabicyclo[3.1.0]hexan-6-yl)-1H- imidazol-4-yl)-3-(trifluoromethyl)- pyridin-2-amine | 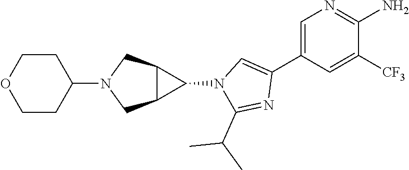 [SMILES: CC(C)c1nc(-c2cnc(N)c(C(F)(F)F)c2)cn1[C@H]1[C@@H]2CN(C3CCOCC3)C[C@@H]21] |
| 14 | VI | 1-(6-(4-(6-amino-5-(trifluoro- methyl)pyridin-3-yl)-2-isopropyl- 1H-imidazol-1-yl)-3-azabicyclo- [3.1.0]hexan-3-yl)ethan-1-one | 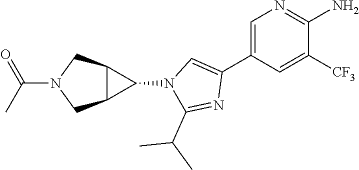 [SMILES: CC(=O)N1C[C@@H]2[C@H](C1)[C@H]2n1cc(-c2cnc(N)c(C(F)(F)F)c2)nc1C(C)C] |
| 15 | VI | 5-(2-isopropyl-1-((1R,5S,6s)-3-(2- methoxyethyl)-3-azabicyclo- [3.1.0]hexan-6-yl)-1H-imidazol-4- yl)-3-(trifluoromethyl)pyridin-2- amine | 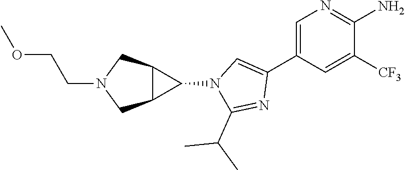 [SMILES: COCCN1C[C@@H]2[C@H](C1)[C@H]2n1cc(-c2cnc(N)c(C(F)(F)F)c2)nc1C(C)C] |
| 16 | VI | 5-(1-((1R,5S,6s)-3-(2-fluoroethyl)- 3-azabicyclo[3.1.0]hexan-6-yl)-2- isopropyl-1H-imidazol-4-yl)-3- (trifluoromethyl)pyridin-2-amine | 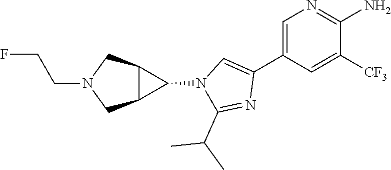 [SMILES: CC(C)c1nc(-c2cnc(N)c(C(F)(F)F)c2)cn1[C@H]1[C@@H]2CN(CCF)C[C@@H]21] |
| 17 | VI | 5-(1-((1R,5S,6s)-3-(2,2-difluoro- ethyl)-3-azabicyclo[3.1.0]hexan-6- yl)-2-isopropyl-1H-imidazol-4-yl)- 3-(trifluoromethyl)pyridin-2- amine | 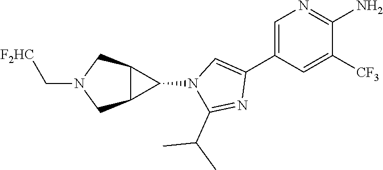 [SMILES: CC(C)c1nc(-c2cnc(N)c(C(F)(F)F)c2)cn1[C@H]1[C@@H]2CN(CC(F)F)C[C@@H]21] |
| 18 | VI | 5-(2-isopropyl-1-((1R,5S,6s)-3- (2,2,2-trifluoroethyl)-3-aza- bicyclo[3.1.0]hexan-6-yl)-1H- imidazol-4-yl)-3-(trifluoromethyl)- pyridin-2-amine | 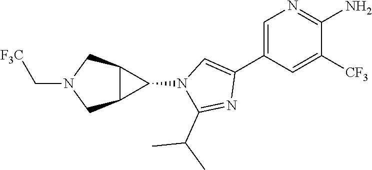 [SMILES: CC(C)c1nc(-c2cnc(N)c(C(F)(F)F)c2)cn1[C@H]1[C@@H]2CN(CC(F)(F)F)C[C@@H]21] |
| 19a | VII | 5-(2-isopropyl-1-((1R,3s,5S,6r)-3- morpholinobicyclo[3.1.0]hexan-6- yl)-1H-imidazol-4-yl)-3-(trifluoro- methoxy)pyridin-2-amine | 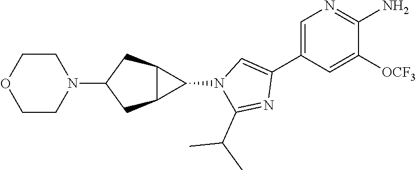 |
| 19b | VII | 5-(2-isopropyl-1-((1R,3r,5S,6r)-3- morpholinobicyclo[3.1.0]hexan-6- yl)-1H-imidazol-4-yl)-3-(trifluoro- methoxy)pyridin-2-amine | 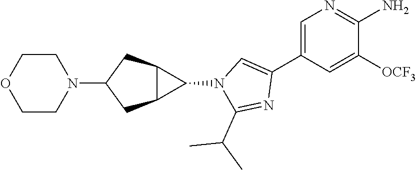 |
| 20a | VII | 5-(1-(3-(1,4-oxazepan-4-yl)- bicyclo[3.1.0]hexan-6-yl)-2- isopropyl-1H-imidazol-4-yl)-3- (trifluoromethoxy)pyridin-2-amine | 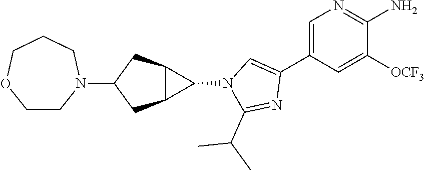 |
| 20b | VII | 5-(1-(3-(1,4-oxazepan-4-yl)- bicyclo[3.1.0]hexan-6-yl)-2- isopropyl-1H-imidazol-4-yl)-3- (trifluoromethoxy)pyridin-2-amine | 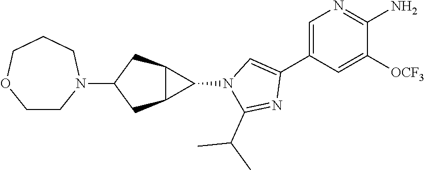 |
| 21 | I | 5-(2-isopropyl-1-methyl-1H- imidazol-4-yl)-3-(trifluoromethyl)- pyridin-2-amine | 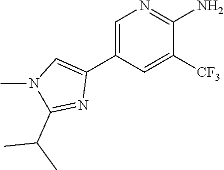 [SMILES: CC(C)c1nc(-c2cnc(N)c(C(F)(F)F)c2)cn1C] |
| 22 | VIII | 1-(4-(6-amino-5-(trifluoromethyl)- pyridin-3-yl)-1-cyclobutyl-1H- imidazol-2-yl)propan-1-ol | 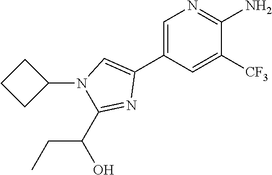 [SMILES: CCC(O)c1nc(-c2cnc(N)c(C(F)(F)F)c2)cn1C1CCC1] |
| 23 | II | 5-(2-isopropyl-1-(2-oxaspiro[3.3]- heptan-6-yl)-1H-imidazol-4-yl)-3- (trifluoromethyl)pyridin-2-amine | 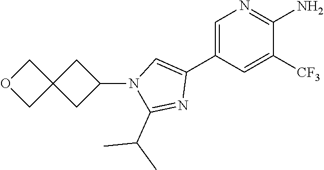 [SMILES: CC(C)c1nc(-c2cnc(N)c(C(F)(F)F)c2)cn1C1CC2(COC2)C1] |
| 24 | II | 5-(1-((1s,3s)-3-fluorocyclobutyl)- 2-isopropyl-1H-imidazol-4-yl)-3- (trifluoromethyl)pyridin-2-amine | 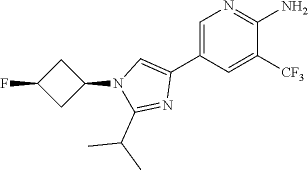 [SMILES: CC(C)c1nc(-c2cnc(N)c(C(F)(F)F)c2)cn1[C@H]1C[C@@H](F)C1] |
| 25 | II | 5-(1-((1r,3r)-3-fluorocyclobutyl)- 2-isopropyl-1H-imidazol-4-yl)-3- (trifluoromethyl)pyridin-2-amine | 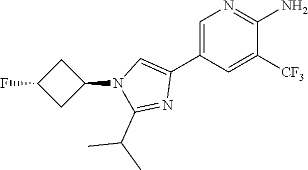 [SMILES: CC(C)c1nc(-c2cnc(N)c(C(F)(F)F)c2)cn1[C@H]1C[C@H](F)C1] |
| 26 | II | 5-(1-(3,3-difluorocyclobutyl)-2- isopropyl-1H-imidazol-4-yl)-3- (trifluoromethyl)pyridin-2-amine | 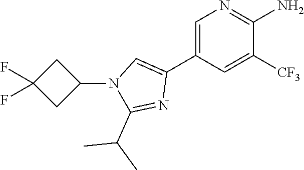 [SMILES: CC(C)c1nc(-c2cnc(N)c(C(F)(F)F)c2)cn1C1CC(F)(F)C1] |
| 27 | II | 5-(2-isopropyl-1-(1-(oxetan-3-yl)- azetidin-3-yl)-1H-imidazol-4-yl)- 3-(trifluoromethyl)pyridin-2- amine | 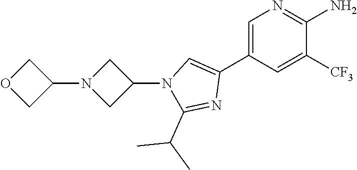 [SMILES: CC(C)c1nc(-c2cnc(N)c(C(F)(F)F)c2)cn1C1CN(C2COC2)C1] |
| 28 | II | 5-(1-cyclopentyl-2-isopropyl-1H- imidazol-4-yl)-3-(trifluoromethyl)- pyridin-2-amine | 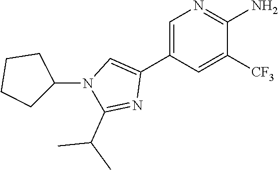 [SMILES: CC(C)c1nc(-c2cnc(N)c(C(F)(F)F)c2)cn1C1CCCC1] |
| 29 | II | 5-(2-isopropyl-1-(oxetan-3-yl)- 1H-imidazol-4-yl)-3-(trifluoro- methyl)pyridin-2-amine | 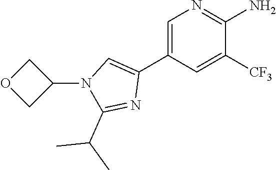 [SMILES: CC(C)c1nc(-c2cnc(N)c(C(F)(F)F)c2)cn1C1COC1] |
| 30 | II | 5-(2-isopropyl-1-(pyridin-2- ylmethyl)-1H-imidazol-4-yl)-3- (trifluoromethyl)pyridin-2-amine | 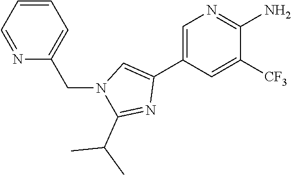 [SMILES: CC(C)c1nc(-c2cnc(N)c(C(F)(F)F)c2)cn1Cc1ccccn1] |
| 31 | II | 5-(2-isopropyl-1-(2-(oxetan-3-yl)- 2-azaspiro[3.3]heptan-6-yl)-1H- imidazol-4-yl)-3-(trifluoromethyl)- pyridin-2-amine | 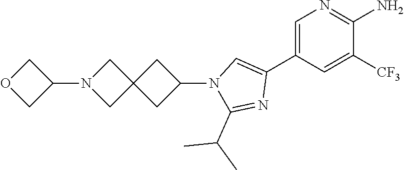 [SMILES: CC(C)c1nc(-c2cnc(N)c(C(F)(F)F)c2)cn1C1CC2(C1)CN(C1COC1)C2] |
| 32 | II | 5-(2-isopropyl-1-(pyridin-3- ylmethyl)-1H-imidazol-4-yl)-3- (trifluoromethyl)pyridin-2-amine | 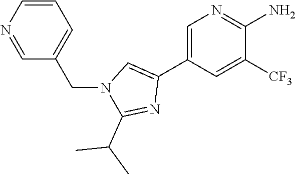 [SMILES: CC(C)c1nc(-c2cnc(N)c(C(F)(F)F)c2)cn1Cc1cccnc1] |
| 33 | II | 4-(4-(6-amino-5-(trifluoromethyl)- pyridin-3-yl)-2-isopropyl-1H- imidazol-1-yl)cyclohexan-1-ol | 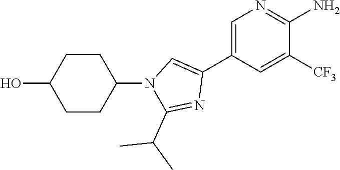 [SMILES: CC(C)c1nc(-c2cnc(N)c(C(F)(F)F)c2)cn1C1CCC(O)CC1] |
| 34 | II | 5-(2-isopropyl-1-((tetrahydro-2H- pyran-4-yl)methyl)-1H-imidazol- 4-yl)-3-(trifluoromethyl)pyridin-2- amine | 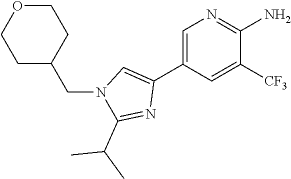 [SMILES: CC(C)c1nc(-c2cnc(N)c(C(F)(F)F)c2)cn1CC1CCOCC1] |
| 35 | II | 5-(2-isopropyl-1-(2-(tetrahydro- 2H-pyran-4-yl)ethyl)-1H- imidazol-4-yl)-3-(trifluoromethyl)- pyridin-2-amine | 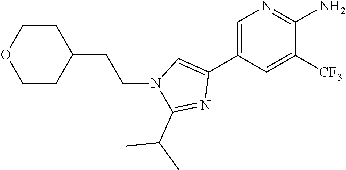 [SMILES: CC(C)c1nc(-c2cnc(N)c(C(F)(F)F)c2)cn1CCC1CCOCC1] |
| 36 | II | 5-(2-isopropyl-1-(1-(oxetan-3-yl)- pyrrolidin-3-yl)-1H-imidazol-4- yl)-3-(trifluoromethyl)pyridin-2- amine | 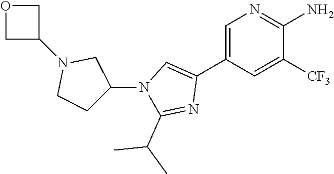 [SMILES: CC(C)c1nc(-c2cnc(N)c(C(F)(F)F)c2)cn1C1CCN(C2COC2)C1] |
| 37 | II | 5-(2-isopropyl-1-(3-morpholino- cyclobutyl)-1H-imidazol-4-yl)-3- (trifluoromethyl)pyridin-2-amine | 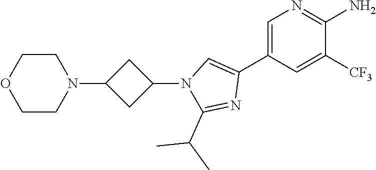 [SMILES: CC(C)c1nc(-c2cnc(N)c(C(F)(F)F)c2)cn1C1CC(N2CCOCC2)C1] |
| 38 | II | 5-(2-isopropyl-1-(6-morpholino- spiro[3.3]heptan-2-yl)-1H- imidazol-4-yl)-3-(trifluoromethyl)- pyridin-2-amine | 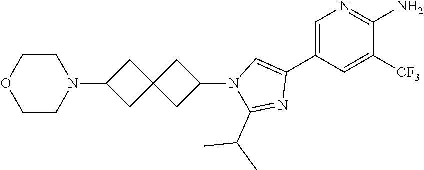 [SMILES: CC(C)c1nc(-c2cnc(N)c(C(F)(F)F)c2)cn1C1CC2(CC(N3CCOCC3)C2)C1] |
| 39 | II | 5-(2-isopropyl-1-(6-morpholino- spiro[3.3]heptan-2-yl)-1H- imidazol-4-yl)-3-(trifluoro- methoxy)pyridin-2-amine | 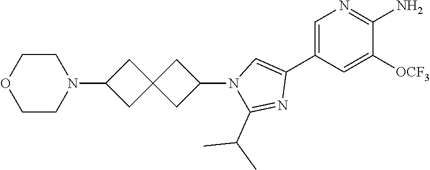 [SMILES: CC(C)c1nc(-c2cnc(N)c(OC(F)(F)F)c2)cn1C1CC2(CC(N3CCOCC3)C2)C1] |
| 40 | II | 5-(2-cyclopropyl-1-(6- morpholinospiro[3.3]heptan-2-yl)- 1H-imidazol-4-yl)-3-(trifluoro- methyl)pyridin-2-amine | 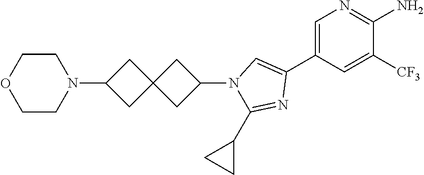 [SMILES: Nc1ncc(-c2cn(C3CC4(CC(N5CCOCC5)C4)C3)c(C3CC3)n2)cc1C(F)(F)F] |
| 41 | II | 5-(2-cyclopropyl-1-(6- morpholinospiro[3.3]heptan-2-yl)- 1H-imidazol-4-yl)-3- (trifluoromethoxy)pyridin-2-amine | 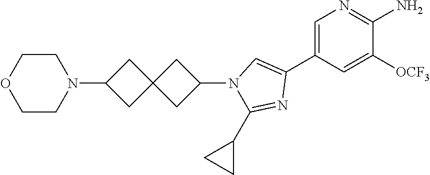 [SMILES: Nc1ncc(-c2cn(C3CC4(CC(N5CCOCC5)C4)C3)c(C3CC3)n2)cc1OC(F)(F)F] |
| 42 | II | 5-(2-isopropyl-1-((1r,3r)-3- morpholinocyclobutyl)-1H- imidazol-4-yl)-3-(trifluoromethyl)- pyridin-2-amine | 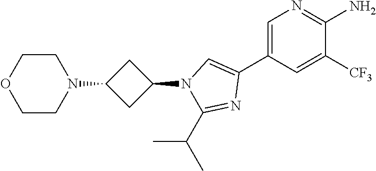 [SMILES: CC(C)c1nc(-c2cnc(N)c(C(F)(F)F)c2)cn1[C@H]1C[C@H](N2CCOCC2)C1] |
| 43 | II | 5-(2-isopropyl-1-((1r,3r)-3- morpholinocyclobutyl)-1H- imidazol-4-yl)-3-(trifluoro- methoxy)pyridin-2-amine | 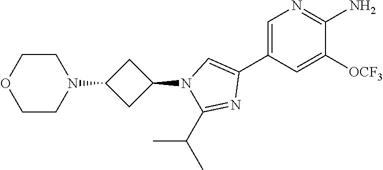 [SMILES: CC(C)c1nc(-c2cnc(N)c(OC(F)(F)F)c2)cn1[C@H]1C[C@H](N2CCOCC2)C1] |
| 44 | II | 5-(2-isopropyl-1-((1s,3s)-3- morpholinocyclobutyl)-1H- imidazol-4-yl)-3-(trifluoromethyl)- pyridin-2-amine | 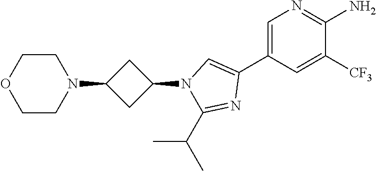 [SMILES: CC(C)c1nc(-c2cnc(N)c(C(F)(F)F)c2)cn1[C@H]1C[C@@H](N2CCOCC2)C1] |
| 45 | II | 5-(1-((1s,3s)-3-(1,4-oxazepan-4- yl)cyclobutyl)-2-isopropyl-1H- imidazol-4-yl)-3-(trifluoromethyl)- pyridin-2-amine | 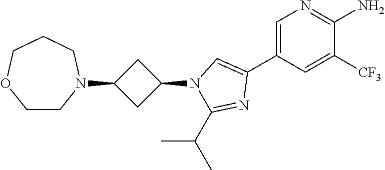 [SMILES: CC(C)c1nc(-c2cnc(N)c(C(F)(F)F)c2)cn1[C@H]1C[C@@H](N2CCCOCC2)C1] |
| 46 | II | 5-(1-cyclopropyl-2-(2,2,2-tri- fluoroethyl)-1H-imidazol-4-yl)-3- (trifluoromethyl)pyridin-2-amine | 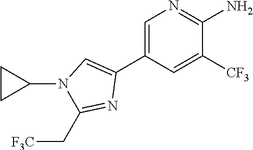 [SMILES: Nc1ncc(-c2cn(C3CC3)c(CC(F)(F)F)n2)cc1C(F)(F)F] |
| 47 | II | 5-(1-cyclopropyl-2-(cyclopropyl- methyl)-1H-imidazol-4-yl)-3- (trifluoromethyl)pyridin-2-amine | 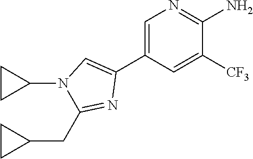 [SMILES: Nc1ncc(-c2cn(C3CC3)c(CC3CC3)n2)cc1C(F)(F)F] |
| 48a | III | 6-(4-(6-amino-5-(trifluoromethyl)- pyridin-3-yl)-2-isopropyl-1H- imidazol-1-yl)bicyclo[3.1.0]- hexan-3-ol | 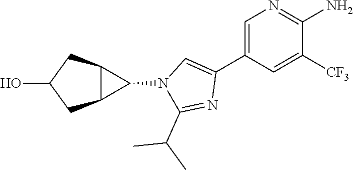 |
| 48b | III | 6-(4-(6-amino-5-(trifluoromethyl)- pyridin-3-yl)-2-isopropyl-1H- imidazol-1-yl)bicyclo[3.1.0]- hexan-3-ol | 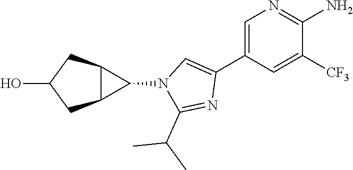 |
| 49 | V | 5-(8-cyclopropyl-5,6-dihydro-8H- imidazo[2,1-c][1,4]oxazin-2-yl)-3- (trifluoromethyl)pyridin-2-amine | 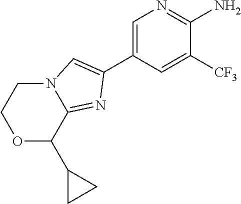 [SMILES: Nc1ncc(-c2cn3c(n2)C(C2CC2)OCC3)cc1C(F)(F)F] |
| 50 | VI | 5-(1-((1R,5S,6s)-3-azabicyclo- [3.1.0]hexan-6-yl)-2-isopropyl- 1H-imidazol-4-yl)-3-(difluoro- methoxy)pyridin-2-amine | 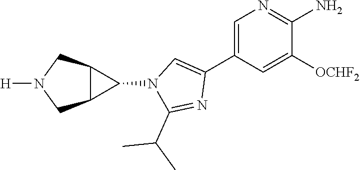 [SMILES: CC(C)c1nc(-c2cnc(N)c(OC(F)F)c2)cn1[C@H]1[C@@H]2CNC[C@@H]21] |
| 51 | VI | 5-(2-cyclobutyl-1-((1R,5S,6s)-3- (oxetan-3-yl)-3-azabicyclo[3.1.0]- hexan-6-yl)-1H-imidazol-4-yl)-3- (trifluoromethyl)pyridin-2-amine | 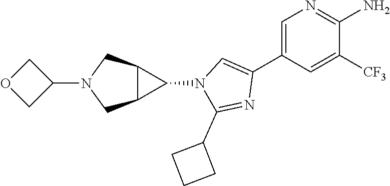 [SMILES: Nc1ncc(-c2cn([C@H]3[C@@H]4CN(C5COC5)C[C@@H]43)c(C3CCC3)n2)cc1C(F)(F)F] |
| 52 | VI | 5-(1-((1R,5S,6s)-3-(oxetan-3-yl)- 3-azabicyclo[3.1.0]hexan-6-yl)-2- (2,2,2-trifluoroethyl)-1H-imidazol- 4-yl)-3-(trifluoromethyl)pyridin-2- amine | 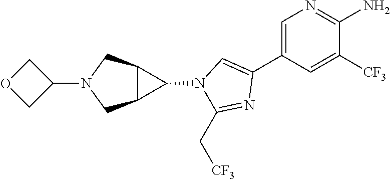 [SMILES: Nc1ncc(-c2cn([C@H]3[C@@H]4CN(C5COC5)C[C@@H]43)c(CC(F)(F)F)n2)cc1C(F)(F)F] |
| 53 | VI | 5-(1-((1R,5S,6s)-3-azabicyclo- [3.1.0]hexan-6-yl)-2-cyclopropyl- 1H-imidazol-4-yl)-3-(trifluoro- methoxy)pyridin-2-amine | 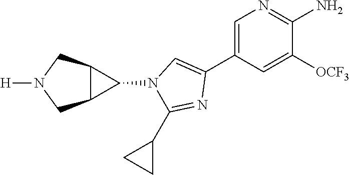 [SMILES: Nc1ncc(-c2cn([C@H]3[C@@H]4CNC[C@@H]43)c(C3CC3)n2)cc1OC(F)(F)F] |
| 54 | VI | 1-((1R,5S)-6-(4-(6-amino-5-(tri- fluoromethoxy)pyridin-3-yl)-2- cyclopropyl-1H-imidazol-1-yl)-3- azabicyclo[3.1.0]hexan-3-yl)- ethan-1-one | 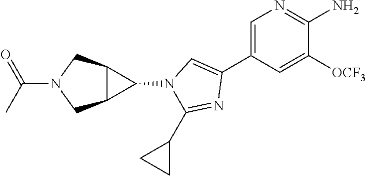 [SMILES: CC(=O)N1C[C@@H]2[C@H](C1)[C@H]2n1cc(-c2cnc(N)c(OC(F)(F)F)c2)nc1C1CC1] |
| 55 | VI | 3-(difluoromethoxy)-5-(2- isopropyl-1-((1R,5S,6s)-3-(2- methoxyethyl)-3-azabicyclo- [3.1.0]hexan-6-yl)-1H-imidazol-4- yl)pyridin-2-amine | 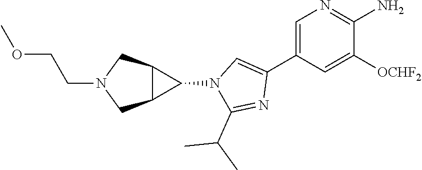 [SMILES: COCCN1C[C@@H]2[C@H](C1)[C@H]2n1cc(-c2cnc(N)c(OC(F)F)c2)nc1C(C)C] |
| 56 | VI | 5-(2-cyclobutyl-1-((1R,5S,6s)-3- (oxetan-3-yl)-3-azabicyclo[3.1.0]- hexan-6-yl)-1H-imidazol-4-yl)-3- (trifluoromethoxy)pyridin-2-amine | 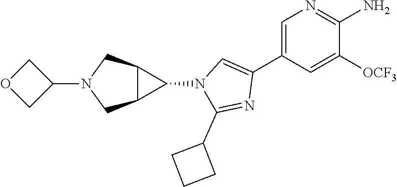 [SMILES: Nc1ncc(-c2cn([C@H]3[C@@H]4CN(C5COC5)C[C@@H]43)c(C3CCC3)n2)cc1OC(F)(F)F] |
| 57 | VI | 5-(2-cyclobutyl-1-((1R,5S,6s)-3- (oxetan-3-yl)-3-azabicyclo[3.1.0]- hexan-6-yl)-1H-imidazol-4-yl)-3- (difluoromethoxy)pyridin-2-amine | 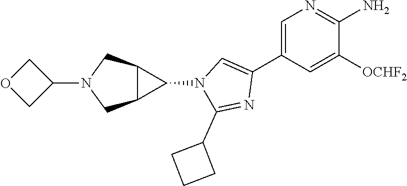 [SMILES: Nc1ncc(-c2cn([C@H]3[C@@H]4CN(C5COC5)C[C@@H]43)c(C3CCC3)n2)cc1OC(F)F] |
| 58 | VI | 5-(1-((1R,5S,6s)-3-azabicyclo- [3.1.0]hexan-6-yl)-2-isopropyl- 1H-imidazol-4-yl)-3-(trifluoro- methoxy)pyridin-2-amine | 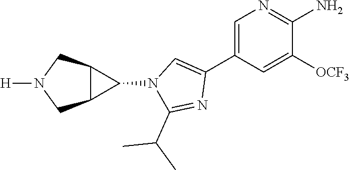 [SMILES: CC(C)c1nc(-c2cnc(N)c(OC(F)(F)F)c2)cn1[C@H]1[C@@H]2CNC[C@@H]21] |
| 59 | VI | 5-(1-((1R,5S,6s)-3-azabicyclo- [3.1.0]hexan-6-yl)-2-(cyclopropyl- methyl)-1H-imidazol-4-yl)-3- (trifluoromethyl)pyridin-2-amine | 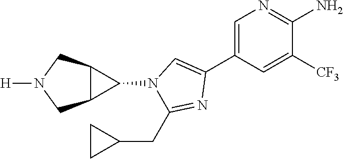 [SMILES: Nc1ncc(-c2cn([C@H]3[C@@H]4CNC[C@@H]43)c(CC3CC3)n2)cc1C(F)(F)F] |
| 60 | VI | 3-(difluoromethoxy)-5-(2- isopropyl-1-((1R,5S,6s)-3- (tetrahydro-2H-pyran-4-yl)-3- azabicyclo[3.1.0]hexan-6-yl)-1H- imidazol-4-yl)pyridin-2-amine | 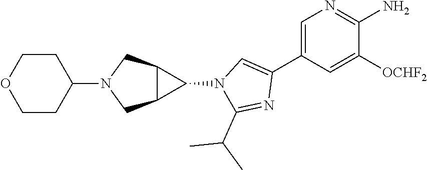 [SMILES: CC(C)c1nc(-c2cnc(N)c(OC(F)F)c2)cn1[C@H]1[C@@H]2CN(C3CCOCC3)C[C@@H]21] |
| 61 | VI | 5-(2-(cyclopropylmethyl)-1- ((1R,5S,6s)-3-(2,2-difluoroethyl)- 3-azabicyclo[3.1.0]hexan-6-yl)- 1H-imidazol-4-yl)-3-(trifluoro- methyl)pyridin-2-amine | 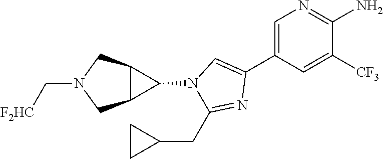 [SMILES: Nc1ncc(-c2cn([C@H]3[C@@H]4CN(CC(F)F)C[C@@H]43)c(CC3CC3)n2)cc1C(F)(F)F] |
| 62 | VI | 5-(2-cyclopentyl-1-((1R,5S,6s)-3- (oxetan-3-yl)-3-azabicyclo[3.1.0]- hexan-6-yl)-1H-imidazol-4-yl)-3- (trifluoromethyl)pyridin-2-amine | 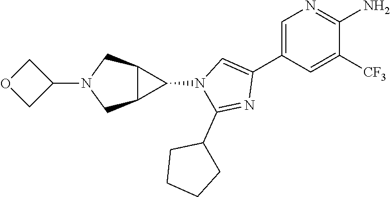 [SMILES: Nc1ncc(-c2cn([C@H]3[C@@H]4CN(C5COC5)C[C@@H]43)c(C3CCCC3)n2)cc1C(F)(F)F] |
| 63 | VI | 5-(2-cyclopentyl-1-((1R,5S,6s)-3- (oxetan-3-yl)-3-azabicyclo[3.1.0]- hexan-6-yl)-1H-imidazol-4-yl)-3- (trifluoromethoxy)pyridin-2-amine | 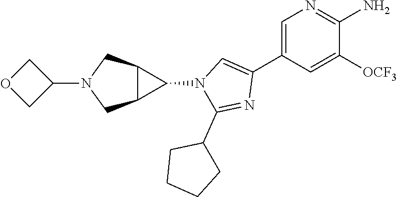 [SMILES: Nc1ncc(-c2cn([C@H]3[C@@H]4CN(C5COC5)C[C@@H]43)c(C3CCCC3)n2)cc1OC(F)(F)F] |
| 64 | VI | 5-(2-cyclopentyl-1-((1R,5S,6s)-3- (oxetan-3-yl)-3-azabicyclo[3.1.0]- hexan-6-yl)-1H-imidazol-4-yl)-3- (difluoromethoxy)pyridin-2-amine | 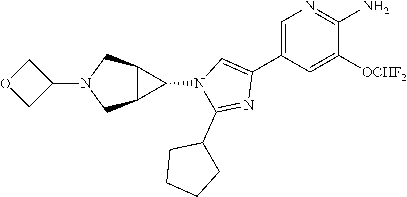 [SMILES: Nc1ncc(-c2cn([C@H]3[C@@H]4CN(C5COC5)C[C@@H]43)c(C3CCCC3)n2)cc1OC(F)F] |
| 65 | VI | 5-(2-cyclopropyl-1-((1R,5S,6s)-3- (oxetan-3-yl)-3-azabicyclo[3.1.0]- hexan-6-yl)-1H-imidazol-4-yl)-3- (trifluoromethoxy)pyridin-2-amine | 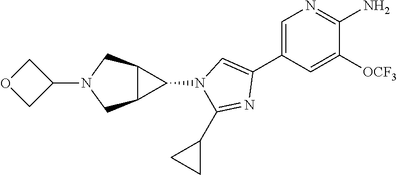 [SMILES: Nc1ncc(-c2cn([C@H]3[C@@H]4CN(C5COC5)C[C@@H]43)c(C3CC3)n2)cc1OC(F)(F)F] |
| 66 | VI | 5-(2-cyclopropyl-1-((1R,5S,6s)-3- (tetrahydro-2H-pyran-4-yl)-3- azabicyclo[3.1.0]hexan-6-yl)-1H- imidazol-4-yl)-3-(trifluoro- methoxy)pyridin-2-amine | 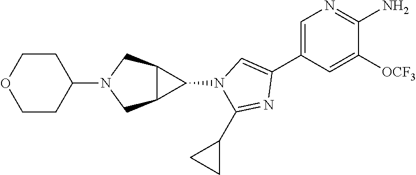 [SMILES: Nc1ncc(-c2cn([C@H]3[C@@H]4CN(C5CCOCC5)C[C@@H]43)c(C3CC3)n2)cc1OC(F)(F)F] |
| 67 | VI | 5-(2-cyclopropyl-1-((1R,5S,6s)-3- (2,2-difluoroethyl)-3-azabicyclo- [3.1.0]hexan-6-yl)-1H-imidazol-4- yl)-3-(trifluoromethoxy)pyridin-2- amine | 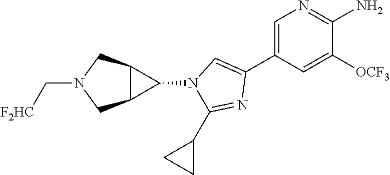 [SMILES: Nc1ncc(-c2cn([C@H]3[C@@H]4CN(CC(F)F)C[C@@H]43)c(C3CC3)n2)cc1OC(F)(F)F] |
| 68 | VI | 5-(2-cyclopropyl-1-((1R,5S,6s)-3- (2-methoxyethyl)-3-azabicyclo- [3.1.0]hexan-6-yl)-1H-imidazol-4- yl)-3-(trifluoromethoxy)pyridin-2- amine | 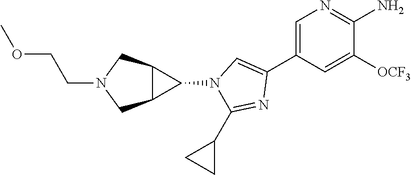 [SMILES: COCCN1C[C@@H]2[C@H](C1)[C@H]2n1cc(-c2cnc(N)c(OC(F)(F)F)c2)nc1C1CC1] |
| 69 | VI | 5-(2-cyclopropyl-1-((1R,5S,6s)-3- (2-fluoroethyl)-3-azabicyclo- [3.1.0]hexan-6-yl)-1H-imidazol-4- yl)-3-(trifluoromethoxy)pyridin-2- amine | 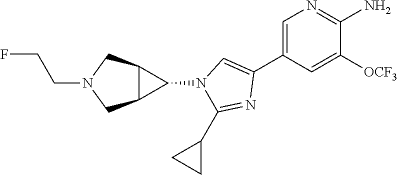 [SMILES: Nc1ncc(-c2cn([C@H]3[C@@H]4CN(CCF)C[C@@H]43)c(C3CC3)n2)cc1OC(F)(F)F] |
| 70 | VI | 5-(2-cyclopropyl-1-((1R,5S,6s)-3- (2,2,2-trifluoroethyl)-3-aza- bicyclo[3.1.0]hexan-6-yl)-1H- imidazol-4-yl)-3-(trifluoro- methoxy)pyridin-2-amine | 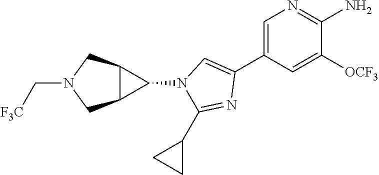 [SMILES: Nc1ncc(-c2cn([C@H]3[C@@H]4CN(CC(F)(F)F)C[C@@H]43)c(C3CC3)n2)cc1OC(F)(F)F] |
| 71 | VI | 5-(1-((1R,5S,6s)-3-azabicyclo- [3.1.0]hexan-6-yl)-2-cyclopropyl- 1H-imidazol-4-yl)-3-(trifluoro- methyl)pyridin-2-amine | 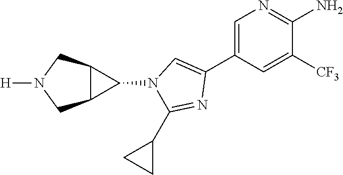 [SMILES: Nc1ncc(-c2cn([C@H]3[C@@H]4CNC[C@@H]43)c(C3CC3)n2)cc1C(F)(F)F] |
| 72 | VI | 1-((1R,5S,6s)-6-(4-(6-amino-5- (trifluoromethyl)pyridin-3-yl)-2- cyclopropyl-1H-imidazol-1-yl)-3- azabicyclo[3.1.0]hexan-3-yl)- ethan-1-one | 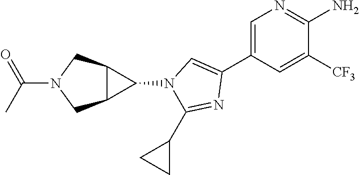 [SMILES: CC(=O)N1C[C@@H]2[C@H](C1)[C@H]2n1cc(-c2cnc(N)c(C(F)(F)F)c2)nc1C1CC1] |
| 73 | VI | 5-(2-cyclopropyl-1-((1R,5S,6s)-3- (oxetan-3-yl)-3-azabicyclo[3.1.0]- hexan-6-yl)-1H-imidazol-4-yl)-3- (trifluoromethyl)pyridin-2-amine | 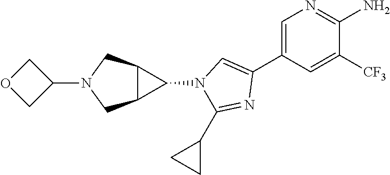 [SMILES: Nc1ncc(-c2cn([C@H]3[C@@H]4CN(C5COC5)C[C@@H]43)c(C3CC3)n2)cc1C(F)(F)F] |
| 74 | VI | 5-(2-cyclopropyl-1-((1R,5S,6s)-3- (tetrahydro-2H-pyran-4-yl)-3- azabicyclo[3.1.0]hexan-6-yl)-1H- imidazol-4-yl)-3-(trifluoro- methyl)pyridin-2-amine | 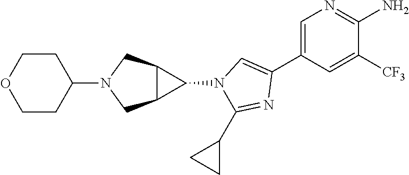 [SMILES: Nc1ncc(-c2cn([C@H]3[C@@H]4CN(C5CCOCC5)C[C@@H]43)c(C3CC3)n2)cc1C(F)(F)F] |
| 75 | VI | 5-(2-cyclopropyl-1-((1R,5S,6s)-3- (2,2-difluoroethyl)-3-azabicyclo- [3.1.0]hexan-6-yl)-1H-imidazol-4- yl)-3-(trifluoromethyl)pyridin-2- amine | 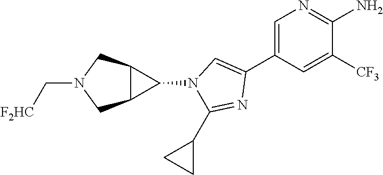 [SMILES: Nc1ncc(-c2cn([C@H]3[C@@H]4CN(CC(F)F)C[C@@H]43)c(C3CC3)n2)cc1C(F)(F)F] |
| 76 | VI | 5-(2-(cyclopropylmethyl)-1- ((1R,5S,6s)-3-(tetrahydro-2H- pyran-4-yl)-3-azabicyclo[3.1.0]- hexan-6-yl)-1H-imidazol-4-yl)-3- (trifluoromethyl)pyridin-2-amine | 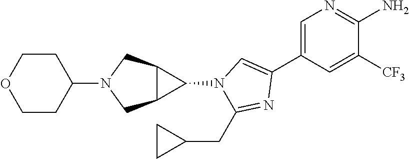 [SMILES: Nc1ncc(-c2cn([C@H]3[C@@H]4CN(C5CCOCC5)C[C@@H]43)c(CC3CC3)n2)cc1C(F)(F)F] |
| 77 | VI | 5-(2-(cyclopropylmethyl)-1- ((1R,5S,6s)-3-(2-fluoroethyl)-3- azabicyclo[3.1.0]hexan-6-yl)-1H- imidazol-4-yl)-3-(trifluoromethyl)- pyridin-2-amine | 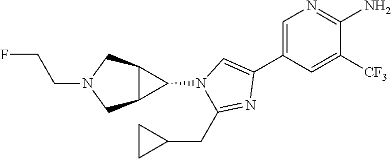 [SMILES: Nc1ncc(-c2cn([C@H]3[C@@H]4CN(CCF)C[C@@H]43)c(CC3CC3)n2)cc1C(F)(F)F] |
| 78 | VI | 5-(2-(cyclopropylmethyl)-1- ((1R,5S,6s)-3-(2-methoxyethyl)-3- azabicyclo[3.1.0]hexan-6-yl)-1H- imidazol-4-yl)-3- (trifluoromethyl)pyridin-2-amine | 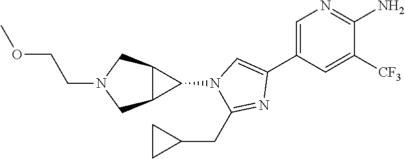 [SMILES: COCCN1C[C@@H]2[C@H](C1)[C@H]2n1cc(-c2cnc(N)c(C(F)(F)F)c2)nc1CC1CC1] |
| 79 | VI | 5-(2-(cyclopropylmethyl)-1- ((1R,5S,6s)-3-(2,2,2-trifluoro- ethyl)-3-azabicyclo[3.1.0]hexan-6- yl)-1H-imidazol-4-yl)-3- (trifluoromethyl)pyridin-2-amine | 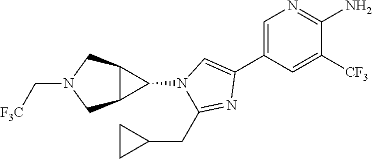 [SMILES: Nc1ncc(-c2cn([C@H]3[C@@H]4CN(CC(F)(F)F)C[C@@H]43)c(CC3CC3)n2)cc1C(F)(F)F] |
| 80 | VI | 5-(2-isopropyl-1-((1R,5S,6s)-3- (oxetan-3-yl)-3-azabicyclo[3.1.0]- hexan-6-yl)-1H-imidazol-4-yl)-3- (trifluoromethoxy)pyridin-2-amine | 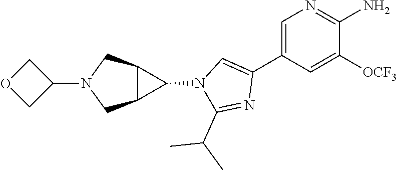 [SMILES: CC(C)c1nc(-c2cnc(N)c(OC(F)(F)F)c2)cn1[C@H]1[C@@H]2CN(C3COC3)C[C@@H]21] |
| 81 | VI | 5-(2-isopropyl-1-((1R,5S,6s)-3- (tetrahydro-2H-pyran-4-yl)-3- azabicyclo[3.1.0]hexan-6-yl)-1H- imidazol-4-yl)-3-(trifluoro- methoxy)pyridin-2-amine | 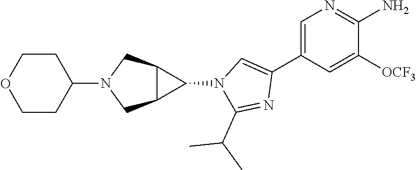 [SMILES: CC(C)c1nc(-c2cnc(N)c(OC(F)(F)F)c2)cn1[C@H]1[C@@H]2CN(C3CCOCC3)C[C@@H]21] |
| 82 | VI | 1-((1R,5S,6s)-6-(4-(6-amino-5- (trifluoromethyl)pyridin-3-yl)-2- (2,2,2-trifluoroethyl)-1H-imidazol- 1-yl)-3-azabicyclo[3.1.0]hexan-3- yl)ethan-1-one | 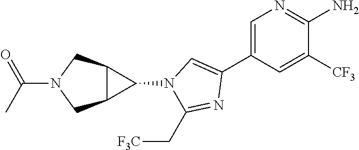 [SMILES: CC(=O)N1C[C@@H]2[C@H](C1)[C@H]2n1cc(-c2cnc(N)c(C(F)(F)F)c2)nc1CC(F)(F)F] |
| 83 | VI | 3-(difluoromethoxy)-5-(2-iso- propyl-1-((1R,5S,6s)-3-(oxetan-3- yl)-3-azabicyclo[3.1.0]hexan-6- yl)-1H-imidazol-4-yl)pyridin-2- amine | 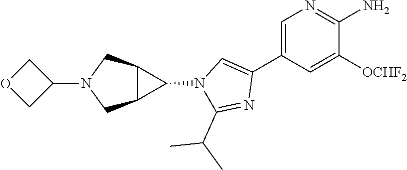 [SMILES: CC(C)c1nc(-c2cnc(N)c(OC(F)F)c2)cn1[C@H]1[C@@H]2CN(C3COC3)C[C@@H]21] |
| 84 | VI | 5-(1-((1R,5S,6s)-3-(2-fluoroethyl)- 3-azabicyclo[3.1.0]hexan-6-yl)-2- propyl-1H-imidazol-4-yl)-3- (trifluoromethyl)pyridin-2-amine | 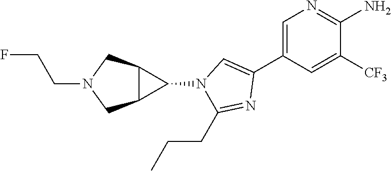 [SMILES: CCCc1nc(-c2cnc(N)c(C(F)(F)F)c2)cn1[C@H]1[C@@H]2CN(CCF)C[C@@H]21] |
| 85 | VI | 5-(1-((1R,5S,6s)-3-(2-fluoroethyl)- 3-azabicyclo[3.1.0]hexan-6-yl)-2- neopentyl-1H-imidazol-4-yl)-3- (trifluoromethyl)pyridin-2-amine | 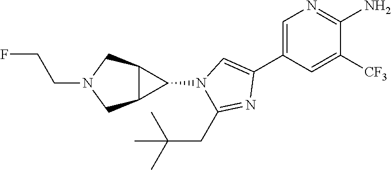 [SMILES: CC(C)(C)Cc1nc(-c2cnc(N)c(C(F)(F)F)c2)cn1[C@H]1[C@@H]2CN(CCF)C[C@@H]21] |
| 86 | VI | 5-(1-((1R,5S,6s)-3-azabicyclo- [3.1.0]hexan-6-yl)-2-(cyclopropyl- methyl)-1H-imidazol-4-yl)-3- (difluoromethoxy)pyridin-2-amine | 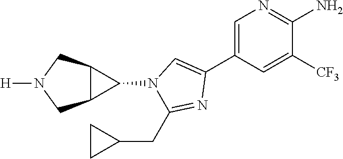 [SMILES: Nc1ncc(-c2cn([C@H]3[C@@H]4CNC[C@@H]43)c(CC3CC3)n2)cc1C(F)(F)F] |
| 87 | VI | 5-(2-(cyclopropylmethyl)-1- ((1R,5S,6s)-3-(oxetan-3-yl)-3- azabicyclo[3.1.0]hexan-6-yl)-1H- imidazol-4-yl)-3-(difluoro- methoxy)pyridin-2-amine | 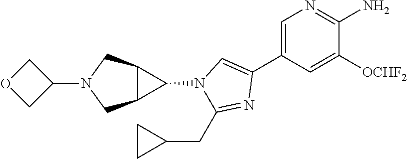 [SMILES: Nc1ncc(-c2cn([C@H]3[C@@H]4CN(C5COC5)C[C@@H]43)c(CC3CC3)n2)cc1OC(F)F] |
| 88 | VI | 5-(2-(cyclopropylmethyl)-1- ((1R,5S,6s)-3-(tetrahydro-2H- pyran-4-yl)-3-azabicyclo[3.1.0]- hexan-6-yl)-1H-imidazol-4-yl)-3- (difluoromethoxy)pyridin-2-amine | 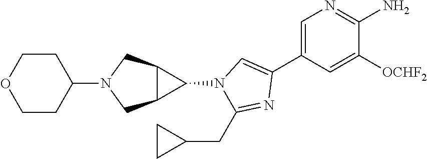 [SMILES: Nc1ncc(-c2cn([C@H]3[C@@H]4CN(C5CCOCC5)C[C@@H]43)c(CC3CC3)n2)cc1OC(F)F] |
| 89 | VI | 5-(2-(cyclopropylmethyl)-1- ((1R,5S,6s)-3-(2,2-difluoroethyl)- 3-azabicyclo[3.1.0]hexan-6-yl)- 1H-imidazol-4-yl)-3-(difluoro- methoxy)pyridin-2-amine | 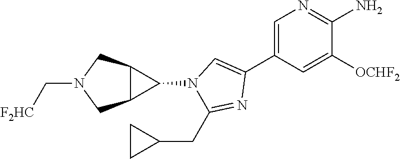 [SMILES: Nc1ncc(-c2cn([C@H]3[C@@H]4CN(CC(F)F)C[C@@H]43)c(CC3CC3)n2)cc1OC(F)F] |
| 90 | VI | 5-(1-((1R,5S,6s)-3-azabicyclo- [3.1.0]hexan-6-yl)-2-(cyclopropyl- methyl)-1H-imidazol-4-yl)-3- (trifluoromethoxy)pyridin-2-amine | 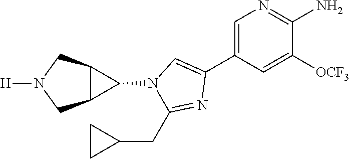 [SMILES: Nc1ncc(-c2cn([C@H]3[C@@H]4CNC[C@@H]43)c(CC3CC3)n2)cc1OC(F)(F)F] |
| 91 | VI | 1-((1R,5S,6s)-6-(4-(6-amino-5- (trifluoromethoxy)pyridin-3-yl)-2- (cyclopropylmethyl)-1H-imidazol- 1-yl)-3-azabicyclo[3.1.0]hexan-3- yl)ethan-1-one | 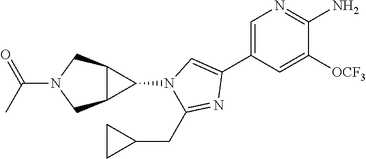 [SMILES: CC(=O)N1C[C@@H]2[C@H](C1)[C@H]2n1cc(-c2cnc(N)c(OC(F)(F)F)c2)nc1CC1CC1] |
| 92 | VI | 5-(2-(cyclopropylmethyl)-1- ((1R,5S,6s)-3-(oxetan-3-yl)-3- azabicyclo[3.1.0]hexan-6-yl)-1H- imidazol-4-yl)-3-(trifluoro- methoxy)pyridin-2-amine | 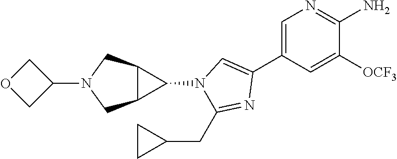 [SMILES: Nc1ncc(-c2cn([C@H]3[C@@H]4CN(C5COC5)C[C@@H]43)c(CC3CC3)n2)cc1OC(F)(F)F] |
| 93 | VI | 5-(2-cyclopropyl-1-((1R,5S,6s)-3- (2,2,2-trifluoroethyl)-3-aza- bicyclo[3.1.0]hexan-6-yl)-1H- imidazol-4-yl)-3-(trifluoromethyl)- pyridin-2-amine | 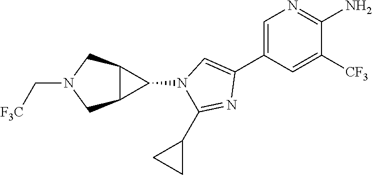 [SMILES: Nc1ncc(-c2cn([C@H]3[C@@H]4CN(CC(F)(F)F)C[C@@H]43)c(C3CC3)n2)cc1C(F)(F)F] |
| 94 | VI | 5-(2-cyclopropyl-1-((1R,5S,6s)-3- (2-methoxyethyl)-3-azabicyclo- [3.1.0]hexan-6-yl)-1H-imidazol-4- yl)-3-(trifluoromethyl)pyridin-2- amine | 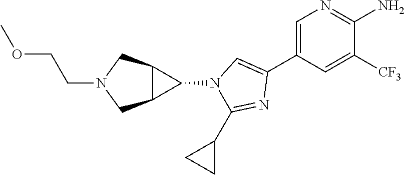 [SMILES: COCCN1C[C@@H]2[C@H](C1)[C@H]2n1cc(-c2cnc(N)c(C(F)(F)F)c2)nc1C1CC1] |
| 95 | VI | 5-(2-cyclopropyl-1-((1R,5S,6s)-3- (2-fluoroethyl)-3-azabicyclo- [3.1.0]hexan-6-yl)-1H-imidazol-4- yl)-3-(trifluoromethyl)pyridin-2- amine | 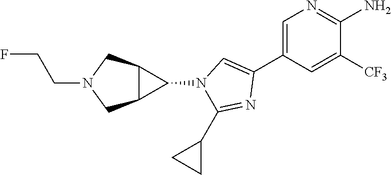 [SMILES: Nc1ncc(-c2cn([C@H]3[C@@H]4CN(CCF)C[C@@H]43)c(C3CC3)n2)cc1C(F)(F)F] |
| 96 | VI | 5-(1-((1R,5S,6s)-3-azabicyclo- [3.1.0]hexan-6-yl)-2-cyclopropyl- 1H-imidazol-4-yl)-3-(difluoro- methoxy)pyridin-2-amine | 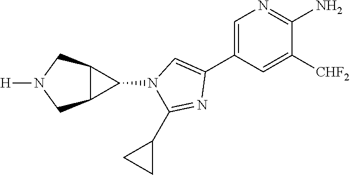 [SMILES: Nc1ncc(-c2cn([C@H]3[C@@H]4CNC[C@@H]43)c(C3CC3)n2)cc1C(F)F] |
| 97 | VI | 5-(2-(cyclopropylmethyl)-1- ((1R,5S,6s)-3-(tetrahydro-2H- pyran-4-yl)-3-azabicyclo[3.1.0]- hexan-6-yl)-1H-imidazol-4-yl)-3- (trifluoromethoxy)pyridin-2-amine | 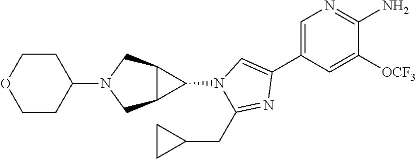 [SMILES: Nc1ncc(-c2cn([C@H]3[C@@H]4CN(C5CCOCC5)C[C@@H]43)c(CC3CC3)n2)cc1OC(F)(F)F] |
| 98 | VI | 5-(2-(cyclopropylmethyl)-1- ((1R,5S,6s)-3-(2,2-difluoroethyl)- 3-azabicyclo[3.1.0]hexan-6-yl)- 1H-imidazol-4-yl)-3- (trifluoromethoxy)pyridin-2-amine | 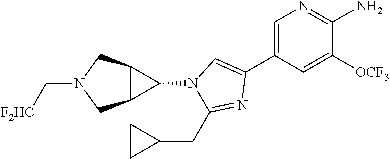 [SMILES: Nc1ncc(-c2cn([C@H]3[C@@H]4CN(CC(F)F)C[C@@H]43)c(CC3CC3)n2)cc1OC(F)(F)F] |
| 99 | VI | 5-(2-(cyclopropylmethyl)-1- ((1R,5S,6s)-3-(2,2,2-trifluoro- ethyl)-3-azabicyclo[3.1.0]hexan-6- yl)-1H-imidazol-4-yl)-3-(trifluoro- methoxy)pyridin-2-amine | 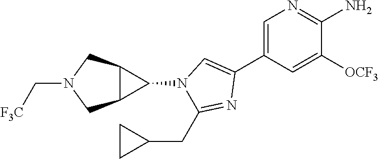 [SMILES: Nc1ncc(-c2cn([C@H]3[C@@H]4CN(CC(F)(F)F)C[C@@H]43)c(CC3CC3)n2)cc1OC(F)(F)F] |
| 100 | VI | 5-(2-(cyclopropylmethyl)-1- ((1R,5S,6s)-3-(2-methoxyethyl)-3- azabicyclo[3.1.0]hexan-6-yl)-1H- imidazol-4-yl)-3-(trifluoro- methoxy)pyridin-2-amine | 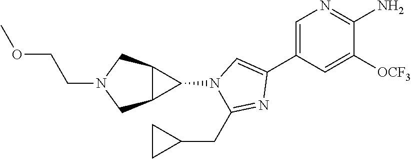 [SMILES: COCCN1C[C@@H]2[C@H](C1)[C@H]2n1cc(-c2cnc(N)c(OC(F)(F)F)c2)nc1CC1CC1] |
| 101 | VI | 5-(2-(cyclopropylmethyl)-1- ((1R,5S,6s)-3-(2-fluoroethyl)-3- azabicyclo[3.1.0]hexan-6-yl)-1H- imidazol-4-yl)-3-(trifluoro- methoxy)pyridin-2-amine | 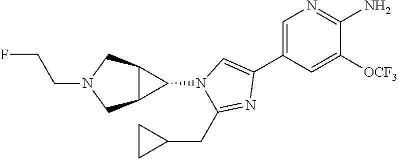 [SMILES: Nc1ncc(-c2cn([C@H]3[C@@H]4CN(CCF)C[C@@H]43)c(CC3CC3)n2)cc1OC(F)(F)F] |
| 102 | VI | 5-(2-(cyclopropylmethyl)-1- ((1R,5S,6s)-3-(2,2,2-trifluoro- ethyl)-3-azabicyclo[3.1.0]hexan-6- yl)-1H-imidazol-4-yl)-3-(difluoro- methoxy)pyridin-2-amine | 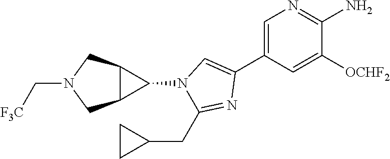 [SMILES: Nc1ncc(-c2cn([C@H]3[C@@H]4CN(CC(F)(F)F)C[C@@H]43)c(CC3CC3)n2)cc1OC(F)F] |
| 103 | VI | 5-(2-cyclopropyl-1-((1R,5S,6s)-3- (2,2-difluoroethyl)-3-azabicyclo- [3.1.0]hexan-6-yl)-1H-imidazol-4- yl)-3-(difluoromethoxy)pyridin-2- amine | 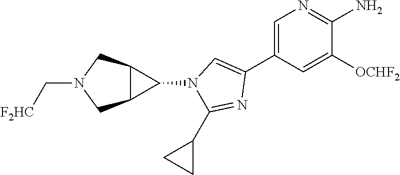 [SMILES: Nc1ncc(-c2cn([C@H]3[C@@H]4CN(CC(F)F)C[C@@H]43)c(C3CC3)n2)cc1OC(F)F] |
| 104 | VI | 5-(2-cyclopropyl-1-((1R,5S,6s)-3- (oxetan-3-yl)-3-azabicyclo[3.1.0]- hexan-6-yl)-1H-imidazol-4-yl)-3- (difluoromethoxy)pyridin-2-amine | 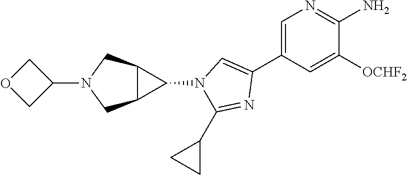 [SMILES: Nc1ncc(-c2cn([C@H]3[C@@H]4CN(C5COC5)C[C@@H]43)c(C3CC3)n2)cc1OC(F)F] |
| 105 | VI | 5-(2-cyclopropyl-1-((1R,5S,6s)-3- (tetrahydro-2H-pyran-4-yl)-3- azabicyclo[3.1.0]hexan-6-yl)-1H- imidazol-4-yl)-3-(difluoro- methoxy)pyridin-2-amine | 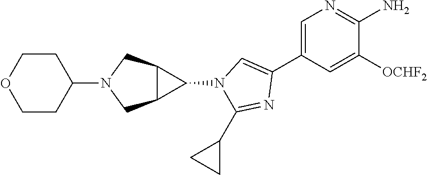 [SMILES: Nc1ncc(-c2cn([C@H]3[C@@H]4CN(C5CCOCC5)C[C@@H]43)c(C3CC3)n2)cc1OC(F)F] |
| 106 | VI | 5-(2-cyclopropyl-1-((1R,5S,6s)-3- (2-fluoroethyl)-3-azabicyclo- [3.1.0]hexan-6-yl)-1H-imidazol-4- yl)-3-(difluoromethoxy)pyridin-2- amine | 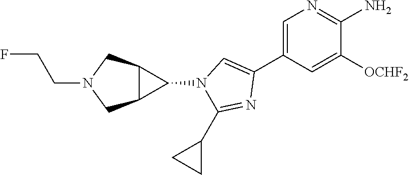 [SMILES: Nc1ncc(-c2cn([C@H]3[C@@H]4CN(CCF)C[C@@H]43)c(C3CC3)n2)cc1OC(F)F] |
| 107 | VI | 5-(2-cyclopropyl-1-((1R,5S,6s)-3- (2-methoxyethyl)-3-azabicyclo- [3.1.0]hexan-6-yl)-1H-imidazol-4- yl)-3-(difluoromethoxy)pyridin-2- amine | 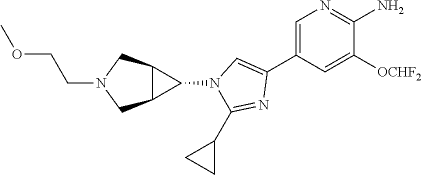 [SMILES: COCCN1C[C@@H]2[C@H](C1)[C@H]2n1cc(-c2cnc(N)c(OC(F)F)c2)nc1C1CC1] |
| 108 | VI | 5-(2-cyclopropyl-1-((1R,5S,6s)-3- (2,2,2-trifluoroethyl)-3-aza- bicyclo[3.1.0]hexan-6-yl)-1H- imidazol-4-yl)-3-(difluoro- methoxy)pyridin-2-amine | 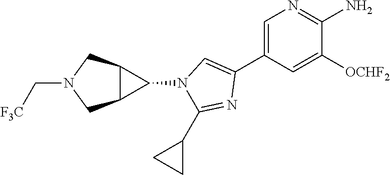 [SMILES: Nc1ncc(-c2cn([C@H]3[C@@H]4CN(CC(F)(F)F)C[C@@H]43)c(C3CC3)n2)cc1OC(F)F] |
| 109 | VI | 5-(1-((1R,5S,6s)-3-(2,2-difluoro- ethyl)-3-azabicyclo[3.1.0]hexan-6- yl)-2-isopropyl-1H-imidazol-4-yl)- 3-(trifluoromethoxy)pyridin-2- amine | 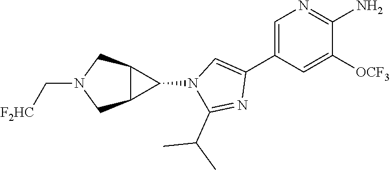 [SMILES: CC(C)c1nc(-c2cnc(N)c(OC(F)(F)F)c2)cn1[C@H]1[C@@H]2CN(CC(F)F)C[C@@H]21] |
| 110 | VI | 5-(2-isopropyl-1-((1R,5S,6s)-3- (2,2,2-trifluoroethyl)-3-aza- bicyclo[3.1.0]hexan-6-yl)-1H- imidazol-4-yl)-3-(trifluoro- methoxy)pyridin-2-amine | 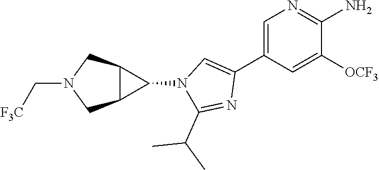 [SMILES: CC(C)c1nc(-c2cnc(N)c(OC(F)(F)F)c2)cn1[C@H]1[C@@H]2CN(CC(F)(F)F)C[C@@H]21] |
| 111 | VI | 5-(1-((1R,5S,6s)-3-(2-fluoroethyl)- 3-azabicyclo[3.1.0]hexan-6-yl)-2- isopropyl-1H-imidazol-4-yl)-3- (trifluoromethoxy)pyridin-2-amine | 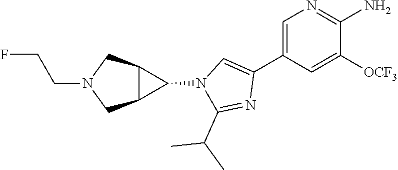 [SMILES: CC(C)c1nc(-c2cnc(N)c(OC(F)(F)F)c2)cn1[C@H]1[C@@H]2CN(CCF)C[C@@H]21] |
| 112 | VI | 5-(1-((1R,5S,6s)-3-(2,2-difluoro- ethyl)-3-azabicyclo[3.1.0]hexan-6- yl)-2-isopropyl-1H-imidazol-4-yl)- 3-(difluoromethoxy)pyridin-2- amine | 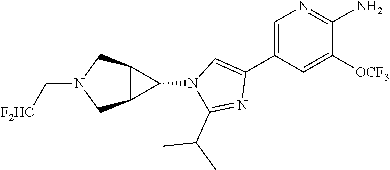 [SMILES: CC(C)c1nc(-c2cnc(N)c(OC(F)(F)F)c2)cn1[C@H]1[C@@H]2CN(CC(F)F)C[C@@H]21] |
| 113 | VI | 3-(difluoromethoxy)-5-(2-iso- propyl-1-((1R,5S,6s)-3-(2- methoxyethyl)-3-azabicyclo- [3.1.0]hexan-6-yl)-1H-imidazol- 4-yl)pyridin-2-amine | 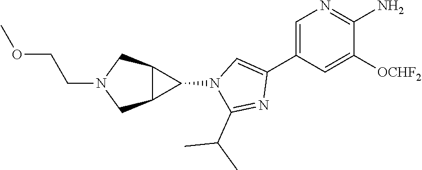 [SMILES: COCCN1C[C@@H]2[C@H](C1)[C@H]2n1cc(-c2cnc(N)c(OC(F)F)c2)nc1C(C)C] |
| 114 | VI | 1-((1R,5S,6s)-6-(4-(6-amino-5- (trifluoromethyl)pyridin-3-yl)-2- (cyclopropylmethyl)-1H-imidazol- 1-yl)-3-azabicyclo[3.1.0]hexan-3- yl)ethan-1-one | 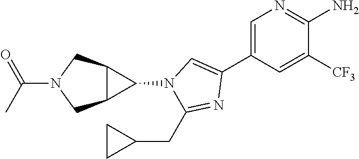 [SMILES: CC(=O)N1C[C@@H]2[C@H](C1)[C@H]2n1cc(-c2cnc(N)c(C(F)(F)F)c2)nc1CC1CC1] |
| 115 | VI | 1-((1R,5S,6s)-6-(4-(6-amino-5- (difluoromethoxy)pyridin-3-yl)-2- (cyclopropylmethyl)-1H-imidazol- 1-yl)-3-azabicyclo[3.1.0]hexan-3- yl)ethan-1-one | 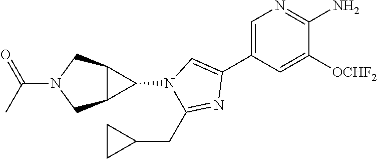 [SMILES: CC(=O)N1C[C@@H]2[C@H](C1)[C@H]2n1cc(-c2cnc(N)c(OC(F)F)c2)nc1CC1CC1] |
| 116 | VI | 5-(2-(cyclopropylmethyl)-1- ((1R,5S,6s)-3-(2-fluoroethyl)-3- azabicyclo[3.1.0]hexan-6-yl)-1H- imidazol-4-yl)-3-(difluoro- methoxy)pyridin-2-amine | 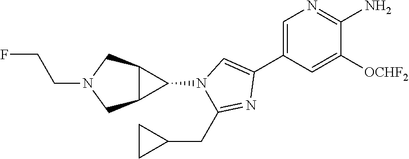 [SMILES: Nc1ncc(-c2cn([C@H]3[C@@H]4CN(CCF)C[C@@H]43)c(CC3CC3)n2)cc1OC(F)F] |
| 117 | VI | 5-(2-(cyclopropylmethyl)-1- ((1R,5S,6s)-3-(2-methoxyethyl)-3- azabicyclo[3.1.0]hexan-6-yl)-1H- imidazol-4-yl)-3-(difluoro- methoxy)pyridin-2-amine | 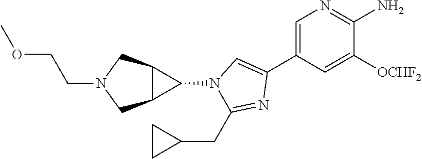 [SMILES: COCCN1C[C@@H]2[C@H](C1)[C@H]2n1cc(-c2cnc(N)c(OC(F)F)c2)nc1CC1CC1] |
| 118 | VI | 1-((1R,5S,6s)-6-(4-(6-amino-5- (difluoromethoxy)pyridin-3-yl)-2- cyclopropyl-1H-imidazol-1-yl)-3- azabicyclo[3.1.0]hexan-3- yl)ethan-1-one | 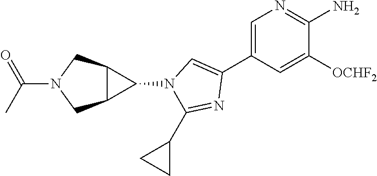 [SMILES: CC(=O)N1C[C@@H]2[C@H](C1)[C@H]2n1cc(-c2cnc(N)c(OC(F)F)c2)nc1C1CC1] |
| 119 | VI | 5-(1-((1R,5S,6s)-3-(tetrahydro- 2H-pyran-4-yl)-3-azabicyclo- [3.1.0]hexan-6-yl)-2-(2,2,2-tri- fluoroethyl)-1H-imidazol-4-yl)-3- (trifluoromethyl)pyridin-2-amine | 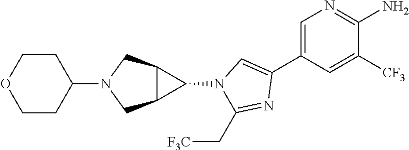 [SMILES: Nc1ncc(-c2cn([C@H]3[C@@H]4CN(C5CCOCC5)C[C@@H]43)c(CC(F)(F)F)n2)cc1C(F)(F)F] |
| 120 | VI | 5-(1-((1R,5S,6s)-3-azabicyclo- [3.1.0]hexan-6-yl)-2-(2,2,2- trifluoroethyl)-1H-imidazol-4-yl)- 3-(trifluoromethyl)pyridin-2- amine | 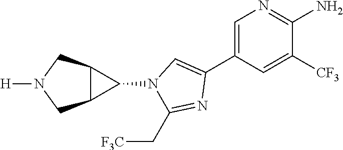 [SMILES: Nc1ncc(-c2cn([C@H]3[C@@H]4CNC[C@@H]43)c(CC(F)(F)F)n2)cc1C(F)(F)F] |
| 121 | VI | 5-(1-((1R,5S,6s)-3-(2-methoxy- ethyl)-3-azabicyclo[3.1.0]hexan-6- yl)-2-(2,2,2-trifluoroethyl)-1H- imidazol-4-yl)-3-(trifluoromethyl)- pyridin-2-amine | 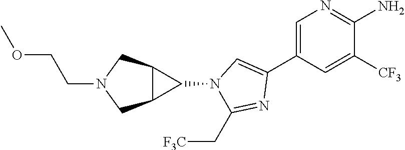 |
| 122 | VI | 5-(1-((1R,5S,6s)-3-(2,2-difluoro- ethyl)-3-azabicyclo[3.1.0]hexan-6- yl)-2-(2,2,2-trifluoroethyl)-1H- imidazol-4-yl)-3-(trifluoro- methyl)pyridin-2-amine | 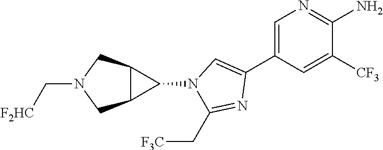 [SMILES: Nc1ncc(-c2cn([C@H]3[C@@H]4CN(CC(F)F)C[C@@H]43)c(CC(F)(F)F)n2)cc1C(F)(F)F] |
| 123 | VI | 5-(1-((1R,5S,6s)-3-(2-fluoroethyl)- 3-azabicyclo[3.1.0]hexan-6-yl)-2- (2,2,2-trifluoroethyl)-1H-imidazol- 4-yl)-3-(trifluoromethyl)pyridin-2- amine | 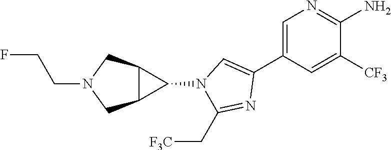 [SMILES: Nc1ncc(-c2cn([C@H]3[C@@H]4CN(CCF)C[C@@H]43)c(CC(F)(F)F)n2)cc1C(F)(F)F] |
| 124 | VI | 5-(2-(2,2,2-trifluoroethyl)-1- ((1R,5S,6s)-3-(2,2,2-trifluoro- ethyl)-3-azabicyclo[3.1.0]hexan-6- yl)-1H-imidazol-4-yl)-3- (trifluoromethyl)pyridin-2-amine | 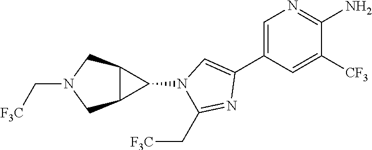 [SMILES: Nc1ncc(-c2cn([C@H]3[C@@H]4CN(CC(F)(F)F)C[C@@H]43)c(CC(F)(F)F)n2)cc1C(F)(F)F] |
| 125 | VI | 5-(1-((1R,5S,6s)-3-(2-fluoroethyl)- 3-azabicyclo[3.1.0]hexan-6-yl)-2- (pentan-3-yl)-1H-imidazol-4-yl)- 3-(trifluoromethyl)pyridin-2- amine | 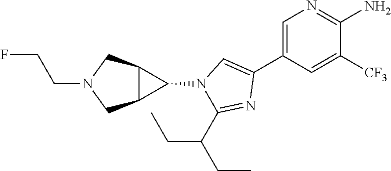 [SMILES: CCC(CC)c1nc(-c2cnc(N)c(C(F)(F)F)c2)cn1[C@H]1[C@@H]2CN(CCF)C[C@@H]21] |
| 126 | VI | 5-(1-((1R,5S,6s)-3-(2-fluoroethyl)- 3-azabicyclo[3.1.0]hexan-6-yl)-2- isobutyl-1H-imidazol-4-yl)-3- (trifluoromethyl)pyridin-2-amine | 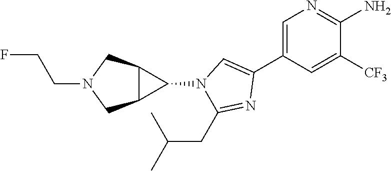 [SMILES: CC(C)Cc1nc(-c2cnc(N)c(C(F)(F)F)c2)cn1[C@H]1[C@@H]2CN(CCF)C[C@@H]21] |
| 127a | VII | 5-(2-isopropyl-1-((1R,3s,5S,6r)-3- morpholinobicyclo[3.1.0]hexan-6- yl)-1H-imidazol-4-yl)-3-(trifluoro- methyl)pyridin-2-amine | 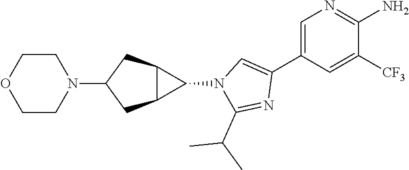 |
| 127b | VII | 5-(2-isopropyl-1-((1R,3r,5S,6r)-3- morpholinobicyclo[3.1.0]hexan-6- yl)-1H-imidazol-4-yl)-3-(trifluoro- methyl)pyridin-2-amine | 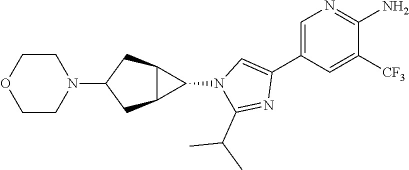 |
| 128a | VII | 5-(2-cyclopropyl-1-(3- morpholinobicyclo[3.1.0]hexan-6- yl)-1H-imidazol-4-yl)-3- (trifluoromethyl)pyridin-2-amine | 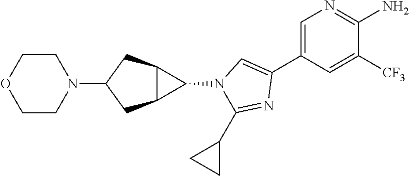 |
| 128b | VII | 5-(2-cyclopropyl-1-(3- morpholinobicyclo[3.1.0]hexan-6- yl)-1H-imidazol-4-yl)-3- (trifluoromethyl)pyridin-2-amine | 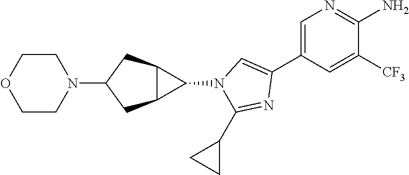 |
| 129a | VII | 5-(2-cyclopropyl-1-(3-(4-methyl- piperazin-1-yl)bicyclo[3.1.0]- hexan-6-yl)-1H-imidazol-4-yl)-3- (trifluoromethyl)pyridin-2-amine | 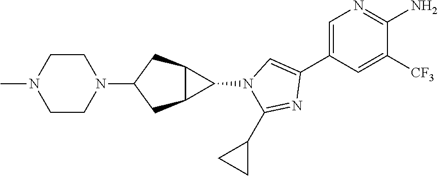 |
| 129b | VII | 5-(2-cyclopropyl-1-(3-(4-methyl- piperazin-1-yl)bicyclo[3.1.0]- hexan-6-yl)-1H-imidazol-4-yl)-3- (trifluoromethyl)pyridin-2-amine | 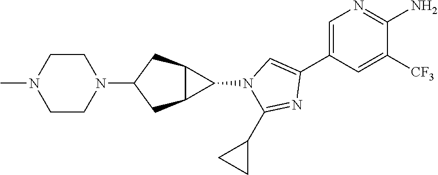 |
| 130 | VII | 5-(1-(3-(1,4-oxazepan-4-yl)- bicyclo[3.1.0]hexan-6-yl)-2- isopropyl-1H-imidazol-4-yl)-3- (trifluoromethyl)pyridin-2-amine | 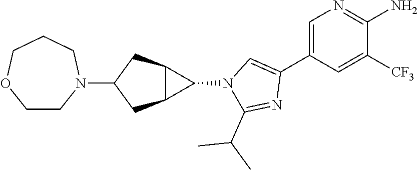 |
| 131 | VII | 5-(2-isopropyl-1-(3-((S)-3- methylmorpholino)bicyclo[3.1.0] hexan-6-yl)-1H-imidazol-4-yl)-3- (trifluoromethyl)pyridin-2-amine | 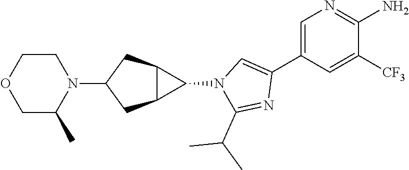 |
| 132 | VII | 5-(2-isopropyl-1-(3-(4-methyl- piperazin-1-yl)bicyclo[3.1.0]- hexan-6-yl)-1H-imidazol-4-yl)-3- (trifluoromethyl)pyridin-2-amine | 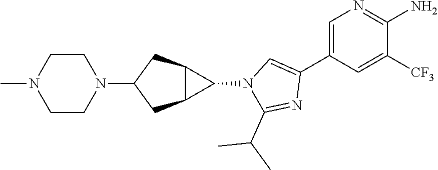 |
| 133a | VII | 5-(2-isopropyl-1-(3-(4-methyl- piperazin-1-yl)bicyclo[3.1.0]- hexan-6-yl)-1H-imidazol-4-yl)-3- (trifluoromethoxy)pyridin-2-amine | 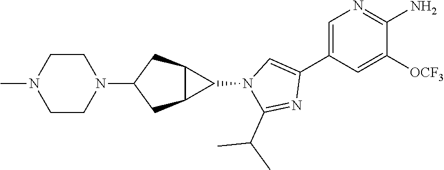 |
| 133b | VII | 5-(2-isopropyl-1-(3-(4-methyl- piperazin-1-yl)bicyclo[3.1.0]- hexan-6-yl)-1H-imidazol-4-yl)-3- (trifluoromethoxy)pyridin-2-amine | 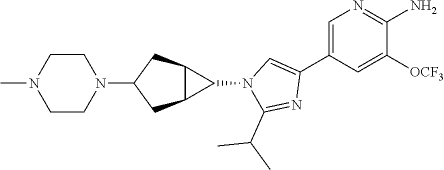 |
| 134 | VII | 5-(1-((1S,5S)-3-((1S,4S)-2-oxa-5- azabicyclo[2.2.1]heptan-5- yl)bicyclo[3.1.0]hexan-6-yl)-2- isopropyl-1H-imidazol-4-yl)-3- (trifluoromethoxy)pyridin-2-amine | 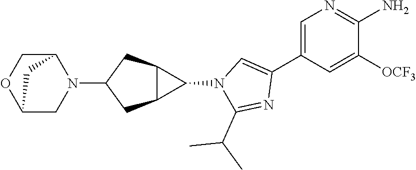 |
| 135 | VII | 5-(2-cyclopropyl-1-(3-(4-methyl- piperazin-1-yl)bicyclo[3.1.0]- hexan-6-yl)-1H-imidazol-4-yl)-3- (trifluoromethoxy)pyridin-2-amine | 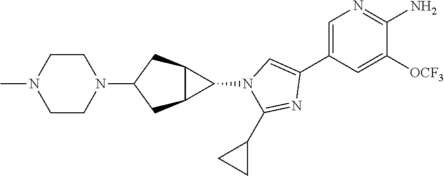 |
| 136 | VII | 5-(1-(3-(1,4-oxazepan-4-yl)- bicyclo[3.1.0]hexan-6-yl)-2- cyclopropyl-1H-imidazol-4-yl)-3- (trifluoromethoxy)pyridin-2-amine | 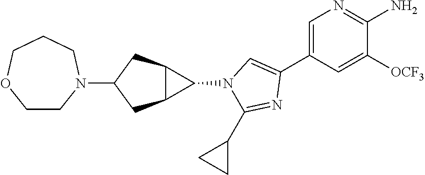 |
| 137 | VII | 5-(2-cyclopropyl-1-(3-(4-methyl- piperazin-1-yl)bicyclo[3.1.0]- hexan-6-yl)-1H-imidazol-4-yl)-3- (difluoromethoxy)pyridin-2-amine | 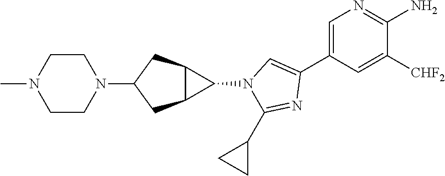 |
| 138a | VII | 5-(2-(cyclopropylmethyl)-1-(3- morpholinobicyclo[3.1.0]hexan-6- yl)-1H-imidazol-4-yl)-3-(trifluoro- methyl)pyridin-2-amine | 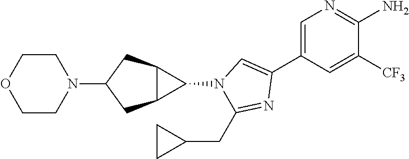 |
| 138b | VII | 5-(2-(cyclopropylmethyl)-1-(3- morpholinobicyclo[3.1.0]hexan-6- yl)-1H-imidazol-4-yl)-3-(trifluoro- methyl)pyridin-2-amine | 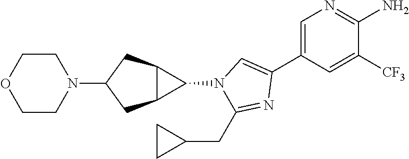 |
| 139a | VII | 5-(2-cyclopropyl-1-(3- morpholinobicyclo[3.1.0]hexan-6- yl)-1H-imidazol-4-yl)-3- (trifluoromethoxy)pyridin-2-amine | 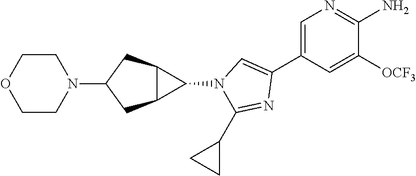 |
| 139b | VII | 5-(2-cyclopropyl-1-(3- morpholinobicyclo[3.1.0]hexan-6- yl)-1H-imidazol-4-yl)-3- (trifluoromethoxy)pyridin-2-amine | 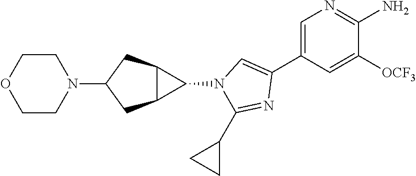 |
| 140a | VII | 5-(2-cyclopropyl-1-(3-((S)-3- methylmorpholino)bicyclo[3.1.0] hexan-6-yl)-1H-imidazol-4-yl)-3- (trifluoromethyl)pyridin-2-amine | 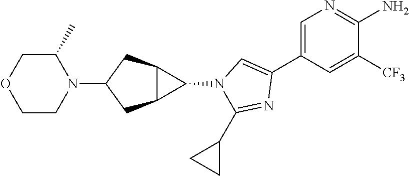 |
| 140b | VII | 5-(2-cyclopropyl-1-(3-((S)-3- methylmorpholino)bicyclo[3.1.0] hexan-6-yl)-1H-imidazol-4-yl)-3- (trifluoromethyl)pyridin-2-amine | 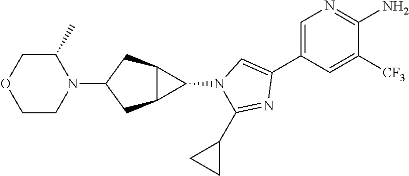 |
| 141a | VII | 5-(1-(3-(1,4-oxazepan-4-yl)- bicyclo[3.1.0]hexan-6-yl)-2- cyclopropyl-1H-imidazol-4-yl)-3- (trifluoromethyl)pyridin-2-amine | 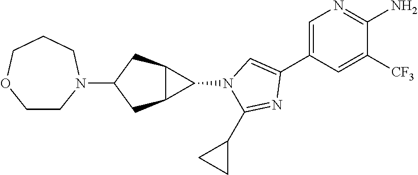 |
| 141b | VII | 5-(1-(3-(1,4-oxazepan-4-yl)- bicyclo[3.1.0]hexan-6-yl)-2- cyclopropyl-1H-imidazol-4-yl)-3- (trifluoromethyl)pyridin-2-amine | 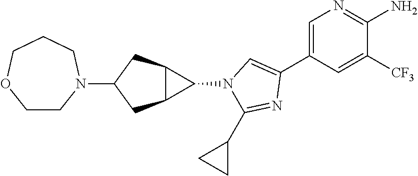 |
| 142 | VII | 5-(1-(3-(1,4-oxazepan-4-yl)- bicyclo[3.1.0]hexan-6-yl)-2- isopropyl-1H-imidazol-4-yl)-3- (trifluoromethyl)pyridin-2-amine | 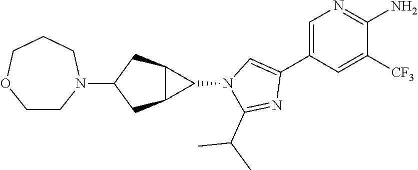 |
| 143 | VII | 5-(2-isopropyl-1-(3-((S)-3- methylmorpholino)bicyclo[3.1.0] hexan-6-yl)-1H-imidazol-4-yl)-3- (trifluoromethyl)pyridin-2-amine | 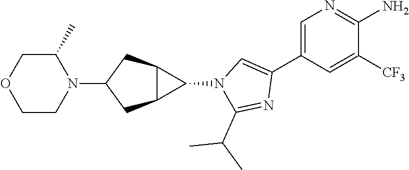 |
| 144 | VII | 5-(2-isopropyl-1-(3-(4-methyl- piperazin-1-yl)bicyclo[3.1.0]- hexan-6-yl)-1H-imidazol-4-yl)-3- (trifluoromethyl)pyridin-2-amine | 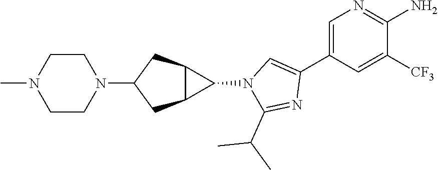 |
| 145a | VII | 3-(difluoromethoxy)-5-(2-iso- propyl-1-(3-(4-methylpiperazin-1- yl)bicyclo[3.1.0]hexan-6-yl)-1H- imidazol-4-yl)pyridin-2-amine | 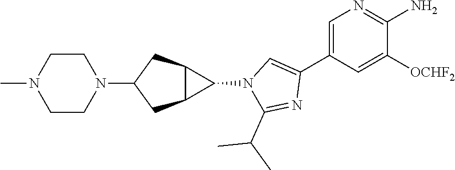 |
| 145b | VII | 3-(difluoromethoxy)-5-(2-iso- propyl-1-(3-(4-methylpiperazin-1- yl)bicyclo[3.1.0]hexan-6-yl)-1H- imidazol-4-yl)pyridin-2-amine | 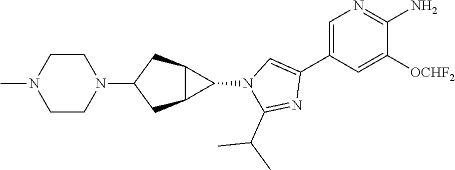 |
| 146a | VII | 5-(2-isobutyl-1-(3-(4-methyl- piperazin-1-yl)bicyclo[3.1.0]- hexan-6-yl)-1H-imidazol-4-yl)-3- (trifluoromethyl)pyridin-2-amine | 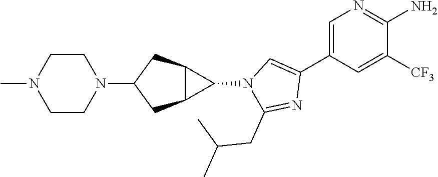 |
| 146b | VII | 5-(2-isobutyl-1-(3-(4-methyl- piperazin-1-yl)bicyclo[3.1.0]- hexan-6-yl)-1H-imidazol-4-yl)-3- (trifluoromethyl)pyridin-2-amine | 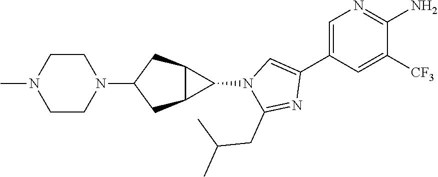 |
| 147 | VII | 5-(2-cyclopropyl-1-(3-(3- methylmorpholino)bicyclo[3.1.0] hexan-6-yl)-1H-imidazol-4-yl)-3- (trifluoromethoxy)pyridin-2-amine | 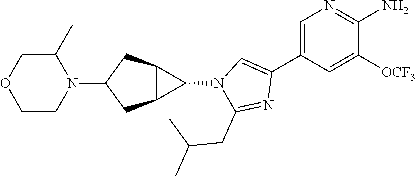 |
| 148 | VII | 5-(2-cyclopropyl-1-(3-(3- methylmorpholino)bicyclo[3.1.0] hexan-6-yl)-1H-imidazol-4-yl)-3- (trifluoromethoxy)pyridin-2-amine | 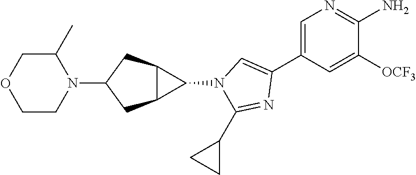 |
| 149 | VII | 5-(1-(3-(1,4-oxazepan-4-yl)- bicyclo[3.1.0]hexan-6-yl)-2- cyclopropyl-1H-imidazol-4-yl)-3- (trifluoromethoxy)pyridin-2-amine | 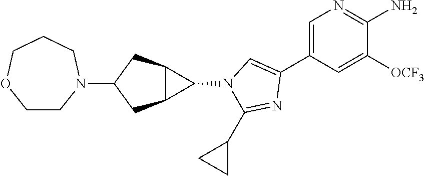 |
| 150 | VII | 5-(2-cyclopropyl-1-(3-(4-methyl- piperazin-1-yl)bicyclo[3.1.0]- hexan-6-yl)-1H-imidazol-4-yl)-3- (difluoromethoxy)pyridin-2-amine | 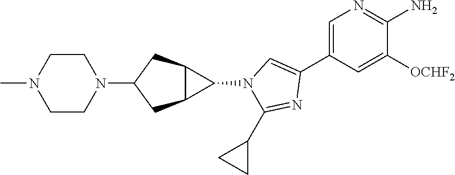 |
| 151a | VII | 5-(2-(cyclopropylmethyl)-1-(3-(4- methylpiperazin-1-yl)bicyclo- [3.1.0]hexan-6-yl)-1H-imidazol-4- yl)-3-(trifluoromethoxy)pyridin-2- amine | 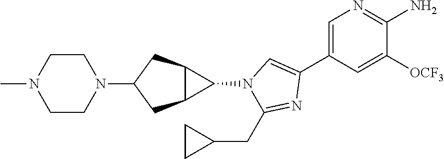 |
| 151b | VII | 5-(2-(cyclopropylmethyl)-1-(3-(4- methylpiperazin-1-yl)bicyclo- [3.1.0]hexan-6-yl)-1H-imidazol-4- yl)-3-(trifluoromethoxy)pyridin-2- amine | 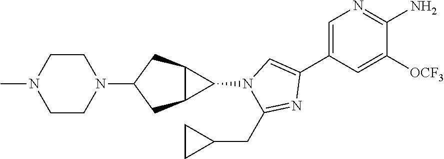 |
| 152a | VII | 5-(2-(cyclopropylmethyl)-1-(3-(4- (2-fluoroethyl)piperazin-1-yl)- bicyclo[3.1.0]hexan-6-yl)-1H- imidazol-4-yl)-3-(trifluoro- methoxy)pyridin-2-amine | 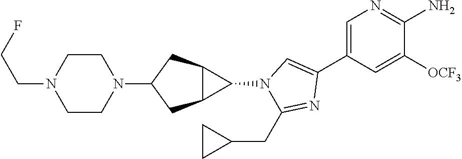 |
| 152b | VII | 5-(2-(cyclopropylmethyl)-1-(3-(4- (2-fluoroethyl)piperazin-1-yl)- bicyclo[3.1.0]hexan-6-yl)-1H- imidazol-4-yl)-3-(trifluoro- methoxy)pyridin-2-amine | 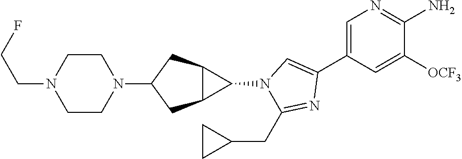 |
| 153a | VII | 5-(2-cyclopropyl-1-(3-(4-(2- fluoroethyl)piperazin-1-yl)- bicyclo[3.1.0]hexan-6-yl)-1H- imidazol-4-yl)-3-(trifluoromethyl)- pyridin-2-amine | 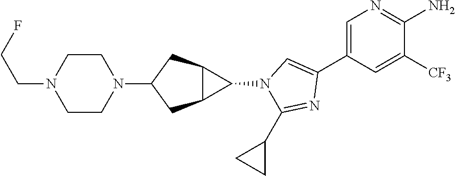 |
| 153b | VII | 5-(2-cyclopropyl-1-(3-(4-(2- fluoroethyl)piperazin-1-yl)- bicyclo[3.1.0]hexan-6-yl)-1H- imidazol-4-yl)-3-(trifluoromethyl)- pyridin-2-amine | 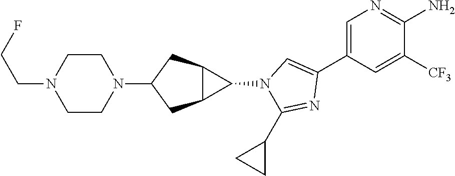 |
| 154a | VII | 5-(2-cyclopropyl-1-(3-(4-(2- fluoroethyl)piperazin-1-yl)- bicyclo[3.1.0]hexan-6-yl)-1H- imidazol-4-yl)-3-(trifluoro- methoxy)pyridin-2-amine | 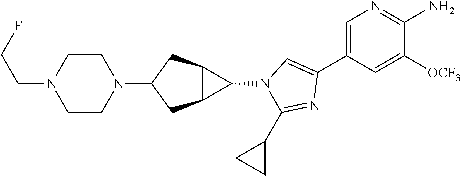 |
| 154b | VII | 5-(2-cyclopropyl-1-(3-(4-(2- fluoroethyl)piperazin-1-yl)- bicyclo[3.1.0]hexan-6-yl)-1H- imidazol-4-yl)-3-(trifluoro- methoxy)pyridin-2-amine | 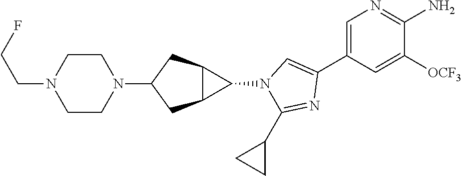 |
| 155a | VII | 5-(1-(3-(4-(2-fluoroethyl)- piperazin-1-yl)bicyclo[3.1.0]- hexan-6-yl)-2-isopropyl-1H- imidazol-4-yl)-3-(trifluoromethyl)- pyridin-2-amine | 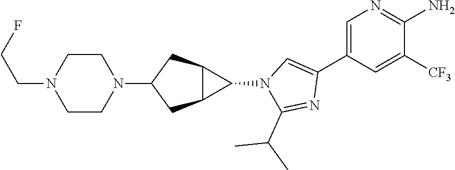 |
| 155b | VII | 5-(1-(3-(4-(2-fluoroethyl)- piperazin-1-yl)bicyclo[3.1.0]- hexan-6-yl)-2-isopropyl-1H- imidazol-4-yl)-3-(trifluoromethyl)- pyridin-2-amine | 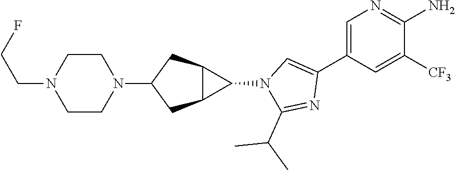 |
| 156a | VII | 5-(1-(3-(4-(2-fluoroethyl)- piperazin-1-yl)bicyclo[3.1.0]- hexan-6-yl)-2-isopropyl-1H- imidazol-4-yl)-3-(trifluoro- methoxy)pyridin-2-amine | 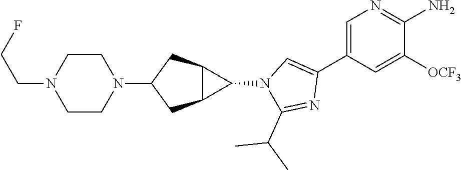 |
| 156b | VII | 5-(1-(3-(4-(2-fluoroethyl)- piperazin-1-yl)bicyclo[3.1.0]- hexan-6-yl)-2-isopropyl-1H- imidazol-4-yl)-3-(trifluoro- methoxy)pyridin-2-amine | 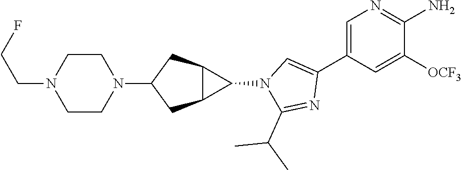 |
| 157a | VII | 5-(2-isopropyl-1-(3-((S)-3- methylmorpholino)bicyclo[3.1.0] hexan-6-yl)-1H-imidazol-4-yl)-3- (trifluoromethoxy)pyridin-2-amine | 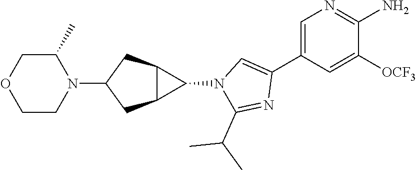 |
| 157b | VII | 5-(2-isopropyl-1-(3-((S)-3- methylmorpholino)bicyclo[3.1.0] hexan-6-yl)-1H-imidazol-4-yl)-3- (trifluoromethoxy)pyridin-2-amine | 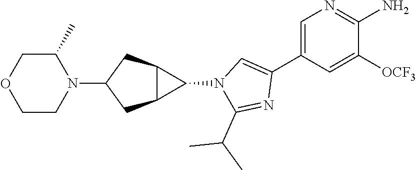 |
| 158 | VII | 5-(2-isobutyl-1-(3-(4-methyl- piperazin-1-yl)bicyclo[3.1.0]- hexan-6-yl)-1H-imidazol-4-yl)-3- (trifluoromethoxy)pyridin-2-amine | 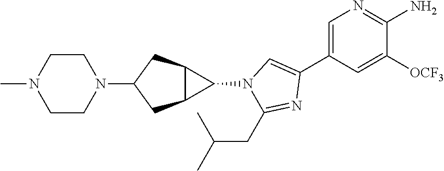 |
| 159a | VII | 5-(1-(3-(4-(2-fluoroethyl)- piperazin-1-yl)bicyclo[3.1.0]- hexan-6-yl)-2-isobutyl-1H- imidazol-4-yl)-3-(trifluoro- methoxy)pyridin-2-amine | 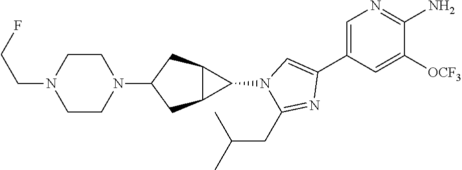 |
| 159b | VII | 5-(1-(3-(4-(2-fluoroethyl)- piperazin-1-yl)bicyclo[3.1.0]- hexan-6-yl)-2-isobutyl-1H- imidazol-4-yl)-3-(trifluoro- methoxy)pyridin-2-amine | 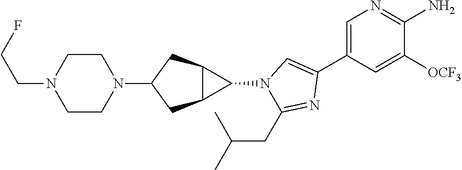 |
| 160a | VII | 5-(1-(3-(4-(2-fluoroethyl)- piperazin-1-yl)bicyclo[3.1.0]- hexan-6-yl)-2-isobutyl-1H- imidazol-4-yl)-3-(trifluoro- methyl)pyridin-2-amine | 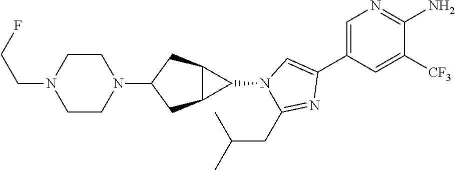 |
| 160b | VII | 5-(1-(3-(4-(2-fluoroethyl)- piperazin-1-yl)bicyclo[3.1.0]- hexan-6-yl)-2-isobutyl-1H- imidazol-4-yl)-3-(trifluoro- methyl)pyridin-2-amine | 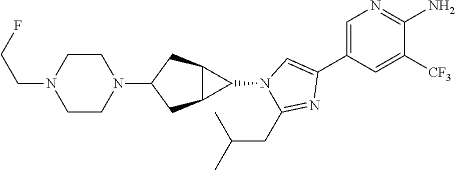 |
| 161a | VII | 5-(2-(cyclopropylmethyl)-1-(3-(4- methylpiperazin-1-yl)bicyclo- [3.1.0]hexan-6-yl)-1H-imidazol-4- yl)-3-(trifluoromethyl)pyridin-2- amine | 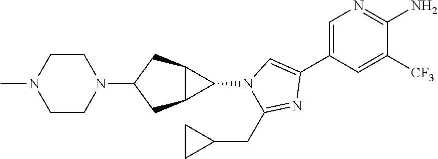 |
| 161b | VII | 5-(2-(cyclopropylmethyl)-1-(3-(4- methylpiperazin-1-yl)bicyclo- [3.1.0]hexan-6-yl)-1H-imidazol-4- yl)-3-(trifluoromethyl)pyridin-2- amine | 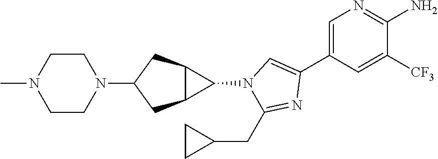 |
| 162a | VII | 5-(2-(cyclopropylmethyl)-1-(3-(4- (2-fluoroethyl)piperazin-1- yl)bicyclo[3.1.0]hexan-6-yl)-1H- imidazol-4-yl)-3-(trifluoromethyl)- pyridin-2-amine | 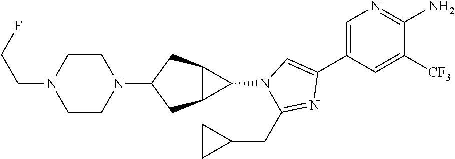 |
| 162b | VII | 5-(2-(cyclopropylmethyl)-1-(3-(4- (2-fluoroethyl)piperazin-1- yl)bicyclo[3.1.0]hexan-6-yl)-1H- imidazol-4-yl)-3-(trifluoromethyl)- pyridin-2-amine | 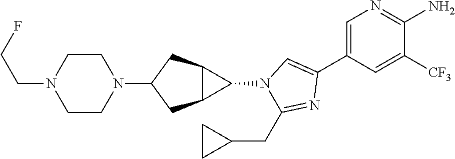 |

TABLE 2
Spectral Data

| | MW | MW |
|---|---|---|
| Ex | calc | obs |
| 1 | 419 | 420 |
| 2 | 419 | 420 |
| 3 | 435 | 436 |
| 4 | 379 | 380 |
| 5 | 298 | 299 |
| 6 | 310 | 311 |
| 7 | 324 | 325 |
| 8 | 310 | 311 |
| 9 | 308 | 309 |
| 10 | 284 | 285 |
| 11 | 351 | 352 |
| 12 | 407 | 408 |
| 13 | 435 | 436 |
| 14 | 393 | 394 |
| 15 | 409 | 410 |
| 16 | 397 | 398 |
| 17 | 415 | 416 |
| 18 | 433 | 434 |
| 19a | 451 | 452 |
| 19b | 451 | 452 |
| 20a | 465 | 466 |
| 20b | 465 | 466 |
| 21 | 284 | 285 |
| 22 | 340 | 341 |
| 23 | 366 | 367 |
| 24 | 342 | 343 |
| 25 | 342 | 343 |
| 26 | 360 | 361 |
| 27 | 381 | 218 |
| 28 | 338 | 339 |
| 29 | 326 | 328 |
| 30 | 361 | 362 |
| 31 | 421 | 422 |
| 32 | 361 | 362 |
| 33 | 368 | 369 |
| 34 | 368 | 369 |
| 35 | 382 | 383 |
| 36 | 395 | 396 |
| 37 | 409 | 410 |
| 38 | 449 | 450 |
| 39 | 465 | 466 |
| 40 | 447 | 448 |
| 41 | 463 | 464 |
| 42 | 409 | 410 |
| 43 | 425 | 426 |
| 44 | 409 | 410 |
| 45 | 423 | 424 |
| 46 | 350 | 351 |
| 47 | 322 | 323 |
| 48a | 366 | 367 |
| 48b | 366 | 367 |
| 49 | 324 | 325 |
| 50 | 349 | 350 |
| 51 | 419 | 420 |
| 52 | 447 | 448 |
| 53 | 365 | 366 |
| 54 | 407 | 408 |
| 55 | 407 | 408 |
| 56 | 435 | 436 |
| 57 | 417 | 418 |
| 58 | 367 | 368 |
| 59 | 363 | 364 |
| 60 | 433 | 434 |
| 61 | 427 | 428 |
| 62 | 433 | 434 |
| 63 | 449 | 450 |
| 64 | 431 | 432 |
| 65 | 421 | 422 |
| 66 | 449 | 450 |
| 67 | 429 | 430 |
| 68 | 423 | 424 |
| 69 | 411 | 412 |
| 70 | 447 | 448 |
| 71 | 349 | 350 |
| 72 | 391 | 392 |
| 73 | 405 | 406 |
| 74 | 433 | 434 |
| 75 | 413 | 414 |
| 76 | 447 | 448 |
| 77 | 409 | 410 |
| 78 | 421 | 422 |
| 79 | 445 | 446 |
| 80 | 423 | 424 |
| 81 | 451 | 452 |
| 82 | 433 | 434 |
| 83 | 405 | 406 |
| 84 | 397 | 398 |
| 85 | 425 | 426 |
| 86 | 361 | 362 |
| 87 | 417 | 418 |
| 88 | 445 | 446 |
| 89 | 425 | 426 |
| 90 | 379 | 380 |
| 91 | 421 | 422 |
| 92 | 435 | 436 |
| 93 | 431 | 432 |
| 94 | 407 | 408 |
| 95 | 395 | 396 |
| 96 | 347 | 348 |
| 97 | 463 | 464 |
| 98 | 443 | 444 |
| 99 | 461 | 462 |
| 100 | 437 | 438 |
| 101 | 425 | 426 |
| 102 | 443 | 444 |
| 103 | 411 | 412 |
| 104 | 403 | 404 |
| 105 | 431 | 432 |
| 106 | 393 | 394 |
| 107 | 405 | 406 |
| 108 | 429 | 430 |
| 109 | 431 | 432 |
| 110 | 449 | 450 |
| 111 | 413 | 414 |
| 112 | 413 | 414 |
| 113 | 407 | 408 |
| 114 | 405 | 406 |
| 115 | 403 | 404 |
| 116 | 407 | 408 |
| 117 | 419 | 420 |
| 118 | 389 | 390 |
| 119 | 475 | 476 |
| 120 | 391 | 392 |
| 121 | 449 | 450 |
| 122 | 455 | 456 |
| 123 | 437 | 438 |
| 124 | 473 | 474 |
| 125 | 425 | 426 |
| 126 | 411 | 412 |
| 127a | 435 | 436 |
| 127b | 435 | 436 |
| 128a | 433 | 434 |
| 128b | 433 | 434 |
| 129a | 446 | 447 |
| 129b | 446 | 447 |
| 130 | 449 | 450 |
| 131 | 449 | 450 |
| 132 | 448 | 449 |
| 133a | 464 | 465 |
| 133b | 464 | 465 |
| 134 | 463 | 464 |
| 135 | 462 | 463 |
| 136 | 463 | 464 |
| 137 | 444 | 445 |
| 138a | 447 | 448 |
| 138b | 447 | 448 |
| 139a | 449 | 450 |
| 139b | 449 | 450 |
| 140a | 447 | 448 |
| 140b | 447 | 448 |
| 141a | 447 | 448 |
| 141b | 447 | 448 |
| 142 | 449 | 450 |
| 143 | 449 | 450 |
| 144 | 448 | 449 |
| 145a | 446 | 447 |
| 145b | 446 | 447 |
| 146a | 462 | 463 |
| 146b | 462 | 463 |
| 147 | 463 | 464 |
| 148 | 463 | 464 |
| 149 | 463 | 464 |
| 150 | 444 | 445 |
| 151a | 476 | 477 |
| 151b | 476 | 477 |
| 152a | 508 | 509 |
| 152b | 508 | 509 |
| 153a | 478 | 479 |
| 153b | 478 | 479 |
| 154a | 494 | 495 |
| 154b | 494 | 495 |
| 155a | 480 | 481 |
| 155b | 480 | 481 |
| 156a | 496 | 497 |
| 156b | 496 | 497 |
| 157a | 465 | 466 |
| 157b | 465 | 466 |
| 158 | 478 | 479 |
| 159a | 510 | 511 |
| 159b | 510 | 511 |
| 160a | 494 | 495 |
| 160b | 494 | 495 |
| 161a | 460 | 461 |
| 161b | 460 | 461 |
| 162a | 492 | 493 |
| 162b | 492 | 493 |

### Biological Activity Assays

Compounds described herein have been shown to bind DLK in vitro, and to inhibit phosphorylation of a downstream molecular target in a cellular assay.

### DLK K_{d} Determinations

The DLK dissociation constants (K_{d}) have been determined in the KINOMEscan KdELECT Service at DiscoveRx.

A fusion protein of full length of human DLK (amino acids 1-859) and the DNA binding domain of NFkB was expressed in transiently transfected HEK293 cells. From these HEK 293 cells, extracts were prepared in M-PER extraction buffer (Pierce) in the presence of Protease Inhibitor Cocktail Complete (Roche) and Phosphatase Inhibitor Cocktail Set II (Merck) per manufacturers' instructions. The DLK fusion protein was labeled with a chimeric double-stranded DNA tag containing the NFkB binding site (5′-GGGAATTCCC-3′) fused to an amplicon for qPCR readout, which was added directly to the expression extract (the final concentration of DNA-tag in the binding reaction is 0.1 nM).

Streptavidin-coated magnetic beads (Dynal M280) were treated with a biotinylated small molecule ligand for 30 minutes at room temperature to generate affinity resins the binding assays. The liganded beads were blocked with excess biotin and washed with blocking buffer (SeaBlock (Pierce), 1% BSA, 0.05% Tween 20, 1 mM DTT) to remove unbound ligand and to reduce nonspecific binding.

The binding reaction was assembled by combining 16 μl of DNA-tagged kinase extract, 3.8 μl liganded affinity beads, and 0.18 μl test compound (PBS/0.05% Tween 20/10 mM DTT/0.1% BSA/2 μg/ml sonicated salmon sperm DNA)]. Extracts were used directly in binding assays without any enzyme purification steps at a ≧10,000-fold overall stock dilution (final DNA-tagged enzyme concentration <0.1 nM). Extracts were loaded with DNA-tag and diluted into the binding reaction in a two step process. First extracts were diluted 1:100 in 1× binding buffer (PBS/0.05% Tween 20/10 mM DTT/0.1% BSA/2 μg/ml sonicated salmon sperm DNA) containing 10 nM DNA-tag. This dilution was allowed to equilibrate at room temperature for 15 minutes and then subsequently diluted 1:100 in 1× binding buffer. Test compounds were prepared as 111× stocks in 100% DMSO. K_{d}s were determined using an 11-point 3-fold compound dilution series with three DMSO control points. All compounds for K_{d} measurements are distributed by acoustic transfer (non-contact dispensing) in 100% DMSO. The compounds were then diluted directly into the assays such that the final concentration of DMSO was 0.9%. All reactions performed in polypropylene 384-well plates. Each was a final volume of 0.02 mL. Assays were incubated with shaking for 1 hour at room temperature. Then the beads were pelleted and washed with wash buffer (1×PBS, 0.05% Tween 20) to remove displaced kinase and test compound. The washed based were re-suspended in elution buffer (1×PBS, 0.05% Tween 20, 0.5 μM non-biotinylated affinity ligand) and incubated at room temperature with shaking for 30 minutes. The kinase concentration in the eluates was measured by qPCR. qPCR reactions were assembled by adding 2.5 μL of kinase eluate to 7.5 μL of qPCR master mix containing 0.15 μM amplicon primers and 0.15 μM amplicon probe. The qPCR protocol consisted of a 10 minute hot start at 95° C., followed by 35 cycles of 95° C. for 15 seconds, 60° C. for 1 minute.

Test Compound Handling.

Test compounds were prepared as 111× stocks in 100% DMSO. K_{d}s were determined using an 11-point 3-fold compound dilution series with three DMSO control points. All compounds for K_{d} measurements are distributed by acoustic transfer (non-contact dispensing) in 100% DMSO. The compounds were then diluted directly into the assays such that the final concentration of DMSO was 0.9%. The K_{d}s were determined using a compound top concentration of 30,000 nM. K_{d} measurements were performed in duplicate.

Binding Constant (K_{d}) Calculation.

Binding constants (K_{d}s) were calculated with a standard dose-response curve using the Hill equation:

$$Response = Background + \frac{( Signal - Background )}{( 1 + ( \frac{{Kd}^{Hill Slope}}{{Dose}^{Hill Slope}} )}$$

The Hill Slope was set to −1. Curves were fitted using a non-linear least square fit with the Levenberg-Marquardt algorithm (Levenberg, K., A method for the solution of certain non-linear problems in least squares, Q. Appl. Math. 2, 164-168 (1944)). See also Fabian, M. A. et al. A small molecule-kinase interaction map for clinical kinase inhibitors. Nat. Biotechnol. 23, 329-336 (2005); Wodicka, L. M. et al. Activation state-dependent binding of small molecule kinase inhibitors: structural insights from biochemistry. Chem Biol. 17, 1241-9 (2010).

Compounds with lower dissociation constants bind with more affinity to the target. Compounds disclosed herein, particularly (but not exclusively) those with lower dissociation constants, can be expected to inhibit target activity and to be useful in the treatment of DLK-mediated disease.

### Phospho-cJun Cellular Assay

HEK293 cells stably transfected with a Dox-inducible human DLK were plated into a 384-well plate in 20 μl (40,000 cells/well) of DMEM medium (without phenol red) containing 10% fetal bovine serum, 1.5 μg/ml doxycycline and 1 μg/ml puromycin. The cells as negative control were grown in the absence of doxycycline. The plate was incubated at 37° C., 5% CO₂ for 20 h, before DMSO (control) or compounds diluted in medium were added. The cells were incubated at 37° C. for an additional 5 h, followed by lysis and the addition detection antibodies from p-cJun (Ser63) cellular assay kit (Cisbio) per manufacturer protocol. The standard dose response curves were fitted by Genedata Screener software using the variable-slope model: Signal=Signal_{negative control}+(Signal_{DMSO control}−Signal_{negative control})/(1+(IC₅₀/Dose)̂Hill slope). Only signal and dose in the equation were treated as known values.

TABLE 3
DLK K_{d} Determinations

| Ex | Kd, nm |
|---|---|
| 1 | 140 |
| 2 | 46 |
| 3 | 51 |
| 4 | 390 |
| 5 | 3300 |
| 6 | 130 |
| 7 | 66 |
| 8 | 130 |
| 9 | 240 |
| 10 | 840 |
| 11 | 120 |
| 12 | 47 |
| 13 | 78 |
| 14 | 170 |
| 15 | 210 |
| 16 | 68 |
| 17 | 170 |
| 18 | 720 |
| 19a | 76 |
| 19b | 38 |
| 20a | 15 |
| 20b | 44 |
| 21 | 640 |
| 22 | 18 |
| 23 | 220 |
| 24 | 240 |
| 25 | 200 |
| 26 | 400 |
| 27 | 270 |
| 28 | 220 |
| 29 | 1000 |
| 30 | 1500 |
| 31 | 82 |
| 32 | 940 |
| 33 | 130 |
| 34 | 870 |
| 35 | 160 |
| 36 | 350 |
| 37 | 92 |
| 38 | 25 |
| 39 | 30 |
| 40 | 42 |
| 41 | 38 |
| 42 | 180 |
| 43 | 120 |
| 44 | ND? |
| 45 | 340 |
| 46 | 280 |
| 47 | 120 |
| 48a | 84 |
| 48b | 59 |
| 49 | 1200 |
| 50 | 170 |
| 51 | 130 |
| 52 | 100 |
| 53 | 57 |
| 54 | 110 |
| 55 | 240 |
| 56 | 280 |
| 57 | 180 |
| 58 | 94 |
| 59 | 50 |
| 60 | 180 |
| 61 | 140 |
| 62 | 85 |
| 63 | 140 |
| 64 | 120 |
| 65 | 32 |
| 66 | 59 |
| 67 | 130 |
| 68 | 77 |
| 69 | 45 |
| 70 | 510 |
| 71 | 87 |
| 72 | 69 |
| 73 | 56 |
| 74 | 54 |
| 75 | 56 |
| 76 | 51 |
| 77 | 17 |
| 78 | 64 |
| 79 | 180 |
| 80 | 42 |
| 81 | 99 |
| 82 | 97 |
| 83 | 160 |
| 84 | 160 |
| 85 | 60 |
| 86 | 110 |
| 87 | 110 |
| 88 | 71 |
| 89 | 290 |
| 90 | 40 |
| 91 | 77 |
| 92 | 55 |
| 93 | 270 |
| 94 | 61 |
| 95 | 12.3 |
| 96 | 53 |
| 97 | 57 |
| 98 | 93 |
| 99 | 340 |
| 100 | 66 |
| 101 | 81 |
| 102 | 280 |
| 103 | 92 |
| 104 | 66 |
| 105 | 79 |
| 106 | 55 |
| 107 | 74 |
| 108 | 140 |
| 109 | 66 |
| 110 | 280 |
| 111 | 100 |
| 112 | 160 |
| 113 | 95 |
| 114 | 66 |
| 115 | 200 |
| 116 | 53 |
| 117 | 48 |
| 118 | 62 |
| 119 | 71 |
| 120 | 40 |
| 121 | 49 |
| 122 | 120 |
| 123 | 65 |
| 124 | 240 |
| 125 | 120 |
| 126 | 34 |
| 127a | 52 |
| 127b | 61 |
| 128a | 40 |
| 128b | 22 |
| 129a | 12.75 |
| 129b | 16 |
| 130 | 25 |
| 131 | 34 |
| 132 | 22 |
| 133a | 20 |
| 133b | 21 |
| 134 | 33 |
| 135 | 17 |
| 136 | 20 |
| 137 | 22 |
| 138a | 23 |
| 138b | 34 |
| 139a | 37 |
| 139b | N.D. |
| 140a | 32 |
| 140b | 37.5 |
| 141a | 26 |
| 141b | 16 |
| 142 | 9.4 |
| 143 | 19 |
| 144 | 38 |
| 145a | 39 |
| 145b | 50 |
| 146a | 6.5 |
| 146b | 4.5 |
| 147 | 23 |
| 148 | 22 |
| 149 | 37 |
| 150 | 54 |
| 151a | 26 |
| 151b | 20 |
| 152a | 37 |
| 152b | 13 |
| 153a | 22 |
| 153b | 9 |
| 154a | 28 |
| 154b | 17 |
| 155a | 23 |
| 155b | 4.5 |
| 156a | 30 |
| 156b | 11 |
| 157a | 57 |
| 157b | 22 |
| 158 | 12 |
| 159a | 33 |
| 159b | 49 |
| 160a | 13 |
| 160b | 8.7 |
| 161a | 19 |
| 161b | N.D. |
| 162a | N.D. |
| 162b | 5.6 |

TABLE 4
Phospho-cJun Cellular Assay

| | IC50, |
|---|---|
| Ex | nm |
| 2 | 1725 |
| 4 | 14767 |
| 6 | 3615 |
| 7 | 1074.9 |
| 19a | 666 |
| 19b | 493 |
| 20a | 965 |
| 20b | 1055 |
| 22 | 550 |
| 31 | 2153 |
| 38 | 1140 |
| 39 | 771 |
| 40 | 587.5 |
| 41 | 483.5 |
| 42 | 2233 |
| 43 | 804 |
| 44 | 7689 |
| 45 | 6186 |
| 48a | 1588 |
| 48b | 1237 |
| 53 | 802 |
| 65 | 1045 |
| 66 | 827 |
| 68 | 745 |
| 69 | 2441 |
| 72 | 2690 |
| 73 | 1196 |
| 74 | 1349 |
| 76 | 760 |
| 77 | 1491 |
| 78 | 781 |
| 80 | 1674 |
| 81 | 1507 |
| 83 | 3551 |
| 84 | 1609 |
| 85 | 594 |
| 88 | 994 |
| 90 | 1124 |
| 92 | 1819 |
| 94 | 1957 |
| 95 | 1201 |
| 97 | 1901 |
| 105 | 1900 |
| 116 | 1435 |
| 117 | 987 |
| 119 | 1852 |
| 121 | 1595 |
| 125 | 1293 |
| 126 | 823 |
| 127a | 939 |
| 127b | 633 |
| 128a | 748 |
| 128b | 755 |
| 129a | 503 |
| 129b | 633 |
| 130 | 1760 |
| 131 | 1393 |
| 132 | 909 |
| 133a | 896 |
| 133b | 645 |
| 134 | 1042 |
| 135 | 346 |
| 136 | 662 |
| 137 | 710 |
| 138a | 1006 |
| 138b | 1055 |
| 139a | 544 |
| 139b | |
| 140a | 834 |
| 140b | 636 |
| 141a | 655 |
| 141b | 587 |
| 142 | 934 |
| 143 | 1510 |
| 144 | 859 |
| 145a | 1202 |
| 145b | 889 |
| 146a | 376 |
| 146b | 346 |
| 147 | 893 |
| 148 | 710 |
| 149 | 574 |
| 150 | 1090 |
| 151a | 994 |
| 151b | 826 |
| 152a | 1354 |
| 152b | 1153 |
| 153a | 891 |
| 153b | 809 |
| 154a | 761 |
| 154b | 597 |
| 155a | 1187 |
| 155b | 1158 |
| 156a | 747 |
| 156b | 874 |
| 157a | 895 |
| 157b | 827 |
| 158 | 500 |
| 159a | 589 |
| 159b | 1551 |
| 160a | 738 |
| 160b | 775 |
| 161a | 454 |
| 161b | 629 |
| 162a | 1042 |
| 162b | 769 |

All references, patents or applications, U.S. or foreign, cited in the application are hereby incorporated by reference as if written herein in their entireties. Where any inconsistencies arise, material literally disclosed herein controls.

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A compound of structural Formula I: 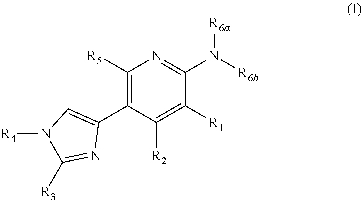
or a salt or ester thereof, wherein:
R₁ is selected from H, halo, alkyl, cycloalkyl, haloalkyl, halocycloalkyl, alkoxy, cycloalkoxy, haloalkoxy, and halocycloalkoxy;
R₂ is selected from H, halo, C₁₋₄ alkyl, and C₁₋₄ alkoxy;
R₃ and R₄ are independently selected from H, alkyl, heteroalkyl, cycloalkyl, heterocycloalkyl, and haloalkyl, any of which is optionally substituted with one to three R₇ groups; or R₃ and R₄ together, in combination with the intervening atoms, form a ring containing atoms selected from C, N, and O, said ring being optionally substituted with one to three R₇ groups;
R₅ is selected from H, halo, C₁₋₄ alkyl, and C₁₋₄ alkoxy;
R₆ₐ and R_{6b} are independently selected from H and C₁₋₄ alkyl;
R₇ is selected from acyl, alkoxy, alkyl, amino, cyano, halo, haloalkyl, haloalkoxy, hydroxyl, sulfonylalkyl, sulfonamidoalkyl, carboxyl, cycloalkyl, heterocycloalkyl, aryl, arylalkyl, and heteroaryl, any of which is optionally substituted with one to three R₈ groups; and
R₈ is selected from C₁₋₄ alkyl, C₁₋₄ alkoxy, halo, hydroxy, oxo, hydroxyalkyl, amino, carboxyl, cyano, C₃₋₆ cycloalkyl, heterocycloalkyl, C₁₋₄ haloalkyl, C₁₋₄ haloalkoxy, aryl, and heteroaryl; or two R₈, in combination with the intervening atoms, form a 4-7 membered ring consisting of atoms selected from C, N, and O, said ring being optionally substituted with one to three groups selected from amino, halo, and hydroxy.

2. The compound of claim 1, or a salt or ester thereof, wherein:
R₃ and R₄ are independently selected from H, alkyl, heteroalkyl, cycloalkyl, heterocycloalkyl, and haloalkyl, any of which is optionally substituted with one to three R₇ groups;

3. The compound of claim 2, wherein R₁ is trifluoromethyl.

4. The compound of claim 3, wherein R₂ and R₅ are H.

5. The compound of claim 4, wherein R₆ₐ and R_{6b} are H.

6. The compound of claim 5, wherein R₃ is selected from bicyclo[3.1.0]hexan-6-yl and 3-azabicyclo[3.1.0]hexan-6-yl, and is optionally substituted with one or more R₇ groups.

7. The compound of claim 6, wherein R₄ is selected from bicyclo[3.1.0]hexan-6-yl and 3-azabicyclo[3.1.0]hexan-6-yl, and is optionally substituted with one or more R₇ groups.

8. The compound of claim 2, wherein the compound has the structural formula III: 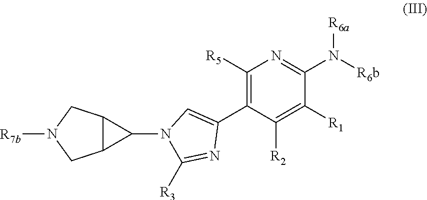
or a salt or ester thereof, wherein:
R₁ is selected from H, halo, alkyl, cycloalkyl, haloalkyl, halocycloalkyl, alkoxy, cycloalkoxy, haloalkoxy, and halocycloalkoxy;
R₂ is selected from H, halo, C₁₋₄ alkyl, and C₁₋₄ alkoxy;
R₃ is selected from H, alkyl, heteroalkyl, cycloalkyl, heterocycloalkyl, and haloalkyl, any of which is optionally substituted with one to three R₇ₐ groups;
R₅ is selected from H, halo, C₁₋₄ alkyl, and C₁₋₄ alkoxy;
R₆ₐ and R_{6b} are independently selected from H and C₁₋₄ alkyl;
R₇ₐ is selected from acyl, alkoxy, alkyl, amino, cyano, halo, haloalkyl, haloalkoxy, hydroxyl, sulfonylalkyl, sulfonamidoalkyl, carboxyl, cycloalkyl, heterocycloalkyl, aryl, arylalkyl, and heteroaryl, any of which is optionally substituted with one to three R₈ groups; and
R_{7b} is selected from H, acyl, alkyl, sulfonylalkyl, sulfonamidoalkyl, carboxyl, cycloalkyl, heterocycloalkyl, aryl, arylalkyl, and heteroaryl, any of which is optionally substituted with one to three R₈ groups; and
R₈ is selected from C₁₋₄alkyl, C₁₋₄ alkoxy, halo, hydroxy, oxo, hydroxyalkyl, amino, carboxyl, cyano, C₃₋₆ cycloalkyl, heterocycloalkyl, C₁₋₄ haloalkyl, C₁₋₄ haloalkoxy, aryl, and heteroaryl; or two R₈, in combination with the intervening atoms, form a 4-7 membered ring consisting of atoms selected from C, N, and O, said ring being optionally substituted with one to three groups selected from amino, halo, and hydroxy.

9. The compound of claim 8, wherein the 7-azabicyclo[3.1.0]heptane ring has exo stereochemistry.

10. The compound of claim 9, wherein R₁ is trifluoromethyl.

11. The compound of claim 10, wherein R₂ and R₅ are H.

12. The compound of claim 11, wherein R₆ₐ and R_{6b} are H.

13. The compound of claim 1, or a salt or ester thereof, wherein R₃ and R₄ together, in combination with the intervening atoms, form a ring, which is optionally substituted with one to three R₇ groups.

14. The compound of claim 1, having structural formula IV: 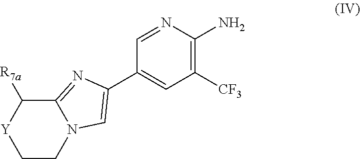
or a salt or ester thereof, wherein:
Y is selected from O, N(R_{7b}), and CH(R_{7b});
R₇ₐ is selected from H, acyl, alkoxy, alkyl, amino, cyano, halo, haloalkyl, haloalkoxy, hydroxyl, sulfonylalkyl, sulfonamidoalkyl, carboxyl, cycloalkyl, heterocycloalkyl, aryl, arylalkyl, and heteroaryl, any of which is optionally substituted with one to three R₈ groups; and
R_{7b} is selected from H, acyl, alkyl, sulfonylalkyl, sulfonamidoalkyl, carboxyl, cycloalkyl, heterocycloalkyl, aryl, arylalkyl, and heteroaryl, any of which is optionally substituted with one to three R₈ groups; and
R₈ is selected from C₁₋₄ alkyl, C₁₋₄ alkoxy, halo, hydroxy, oxo, alkoxy, hydroxyalkyl, amino, carboxyl, cyano, C₃₋₆ cycloalkyl, heterocycloalkyl, C₁₋₄ haloalkyl, C₁₋₄ haloalkoxy, aryl, and heteroaryl; or two R₈, in combination with the intervening atoms, form a 4-7 membered ring consisting of atoms selected from C, N, and O, said ring being optionally substituted with one to three groups selected from amino, halo, and hydroxy.

15. The compound of claim 2, wherein the compound has the structural formula V: 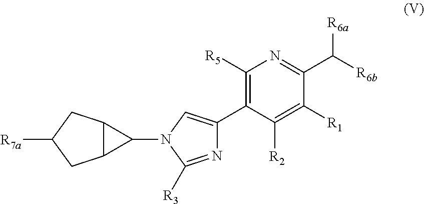
or a salt or ester thereof, wherein:
R₁ is selected from H, halo, alkyl, cycloalkyl, haloalkyl, halocycloalkyl, alkoxy, cycloalkoxy, haloalkoxy, and halocycloalkoxy;
R₂ is selected from H, halo, C₁₋₄ alkyl, and C₁₋₄ alkoxy;
R₃ is selected from H, alkyl, heteroalkyl, cycloalkyl, heterocycloalkyl, and haloalkyl, any of which is optionally substituted with one to three R_{7b} groups;
R₅ is selected from H, halo, C₁₋₄ alkyl, and C₁₋₄ alkoxy;
R₆ₐ and R_{6b} are independently selected from H and C₁₋₄ alkyl;
R₇ₐ and R_{7b} are independently selected from acyl, alkoxy, alkyl, amino, halo, hydroxyl, sulfonylalkyl, sulfonamidoalkyl, carboxyl, cycloalkyl, heterocycloalkyl, aryl, arylalkyl, and heteroaryl, any of which is optionally substituted with one to three R₈ groups; and
R₈ is selected from C₁₋₄ alkyl, C₁₋₄ alkoxy, halo, hydroxy, oxo, hydroxyalkyl, amino, carboxyl, cyano, C₃₋₆ cycloalkyl, heterocycloalkyl, C₁₋₄ haloalkyl, C₁₋₄ haloalkoxy, aryl, and heteroaryl; or two R₈, in combination with the intervening atoms, form a 4-7 membered ring consisting of atoms selected from C, N, and O, said ring being optionally substituted with one to three groups selected from amino, halo, and hydroxy.

16. The compound of claim 15, wherein the bicyclo[3.1.0]heptane ring has exo stereochemistry.

17. The compound of claim 16, wherein R₇ₐ is selected from alkyl, cycloalkyl, and heterocycloalkyl, and is optionally substituted with one to three R₈ groups.

18. The compound of claim 17, wherein R₇ₐ is selected from piperazin-1-yl, morpholin-1-yl, 1,4-diazepan-1-yl, and 1,4-oxazepan-4-yl, and is optionally substituted with one or two R₈ groups.

19. The compound of claim 18, wherein R₁ is trifluoromethyl.

20. The compound of claim 19, wherein R₂ and R₅ are H.

21. The compound of claim 20, wherein R₆ₐ and R_{6b} are H.

22. The compound of claim 21, wherein R₈ is selected from C₁₋₄ alkyl, C₁₋₄ alkoxy, halo, hydroxy, oxo, hydroxyalkyl, amino, carboxyl, cyano, C₃₋₆ cycloalkyl, heterocycloalkyl, C₁₋₄ haloalkyl, C₁₋₄ haloalkoxy, aryl, and heteroaryl.

23. The compound of claim 22, wherein R₈ is selected from C₁₋₄ alkyl, C₁₋₄ alkoxy, halo, hydroxy, oxo, hydroxyalkyl, C₃₋₆ cycloalkyl, heterocycloalkyl, C₁₋₄ haloalkyl, and C₁₋₄ haloalkoxy.

24. The compound of claim 23, wherein R₈ is selected from C₁₋₄ alkyl and C₁₋₄ haloalkyl.

25. The compound of claim 24, wherein R₇ₐ is selected from:

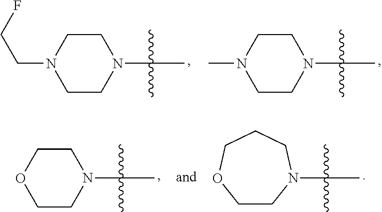

26. The compound of claim 1, wherein the compound is chosen from: 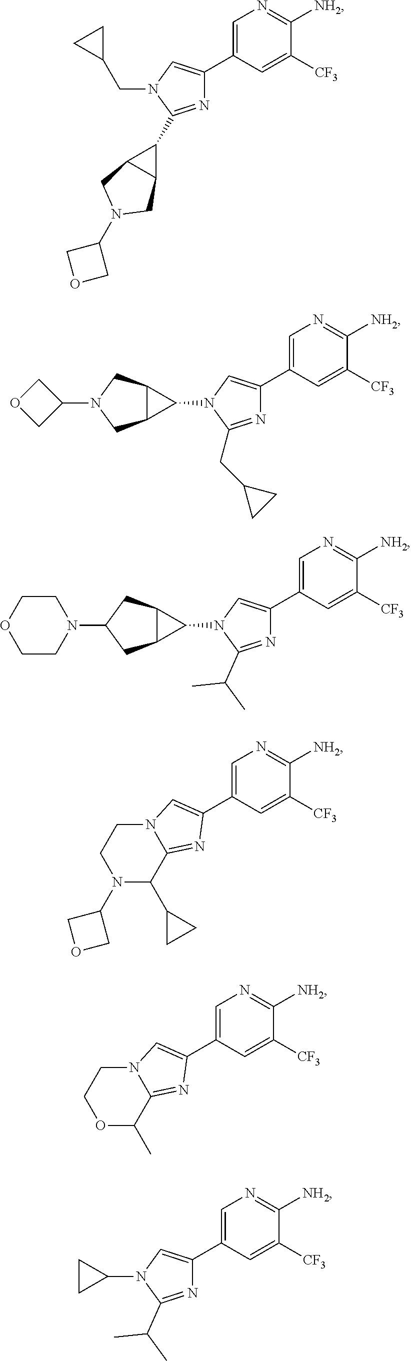 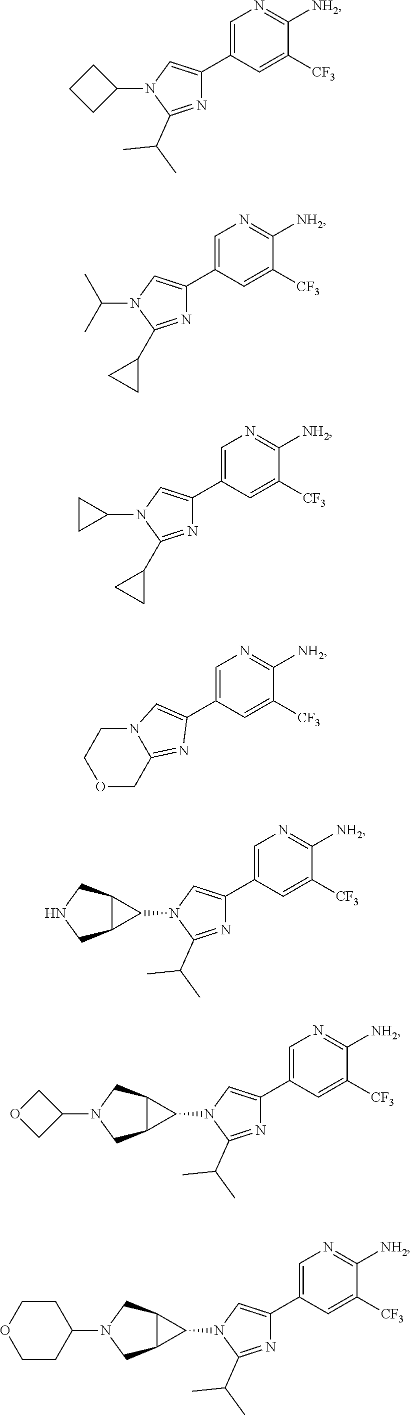 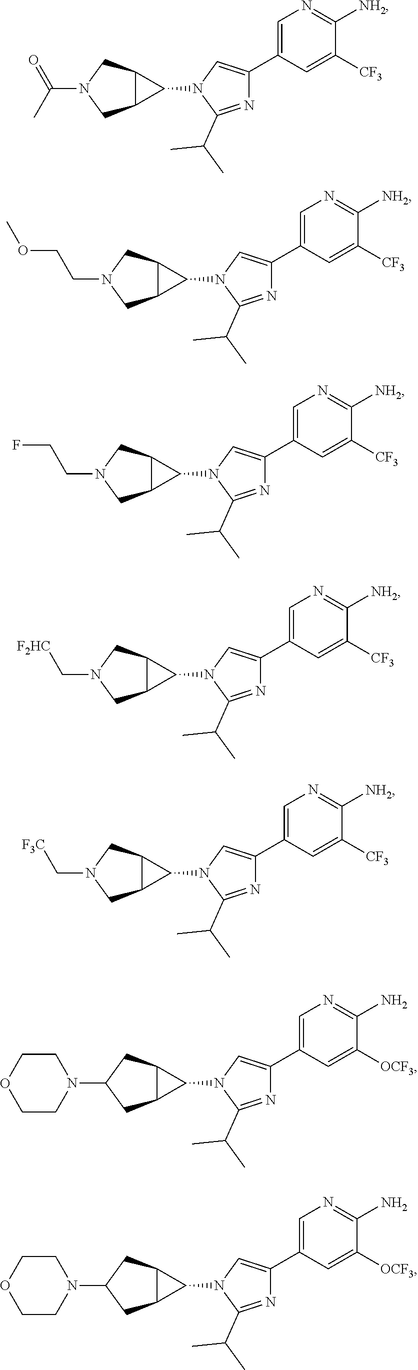 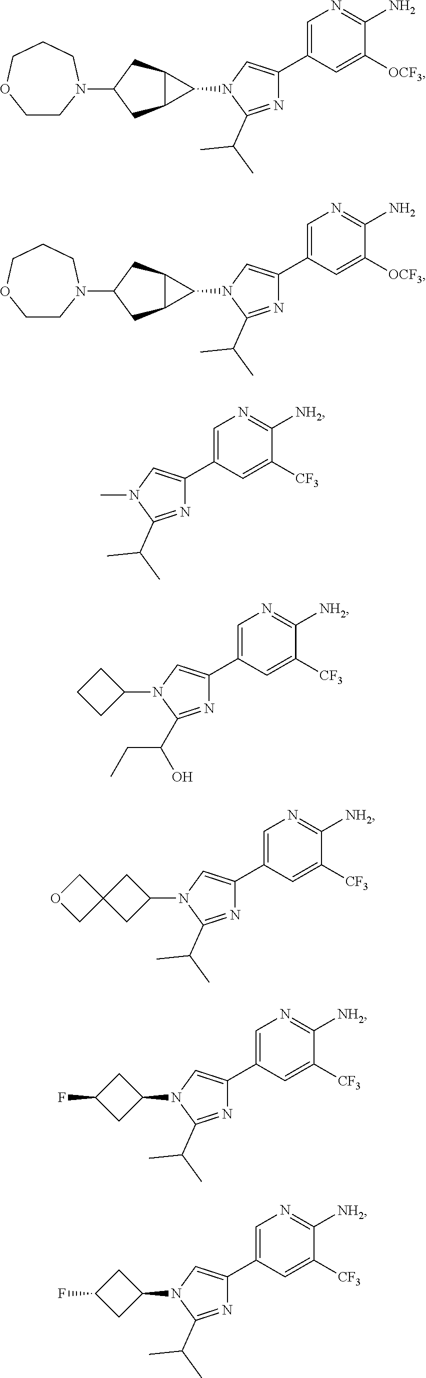 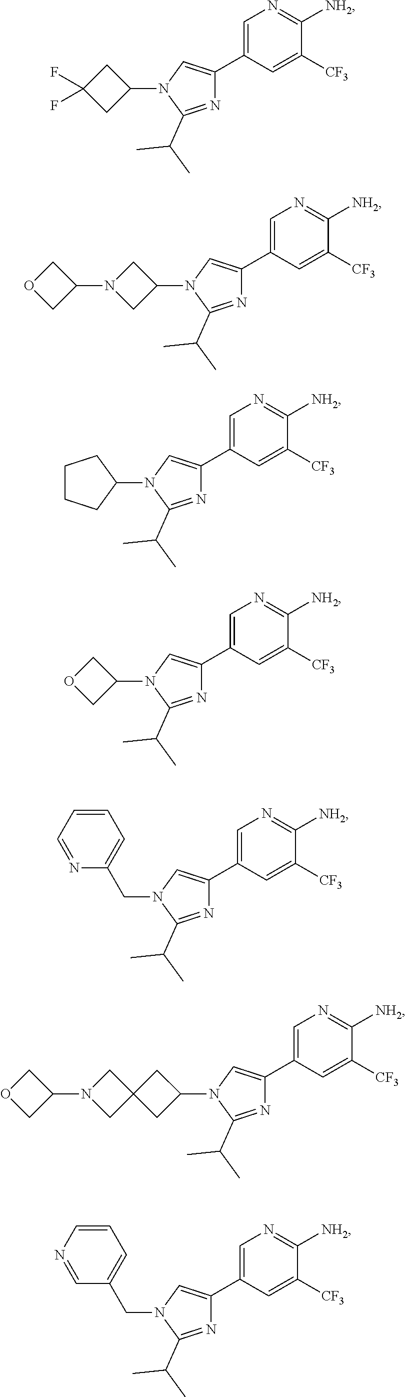 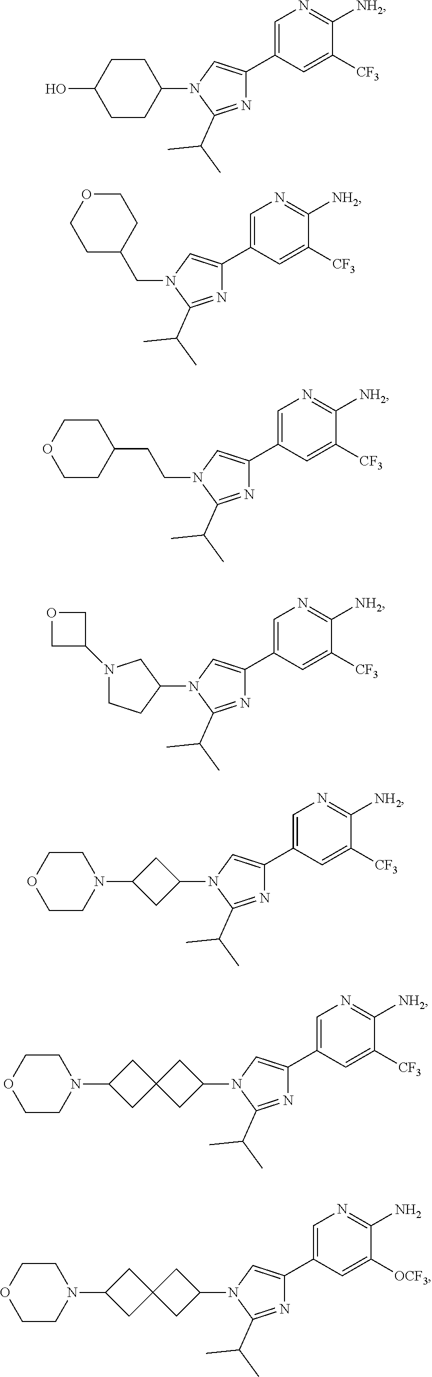 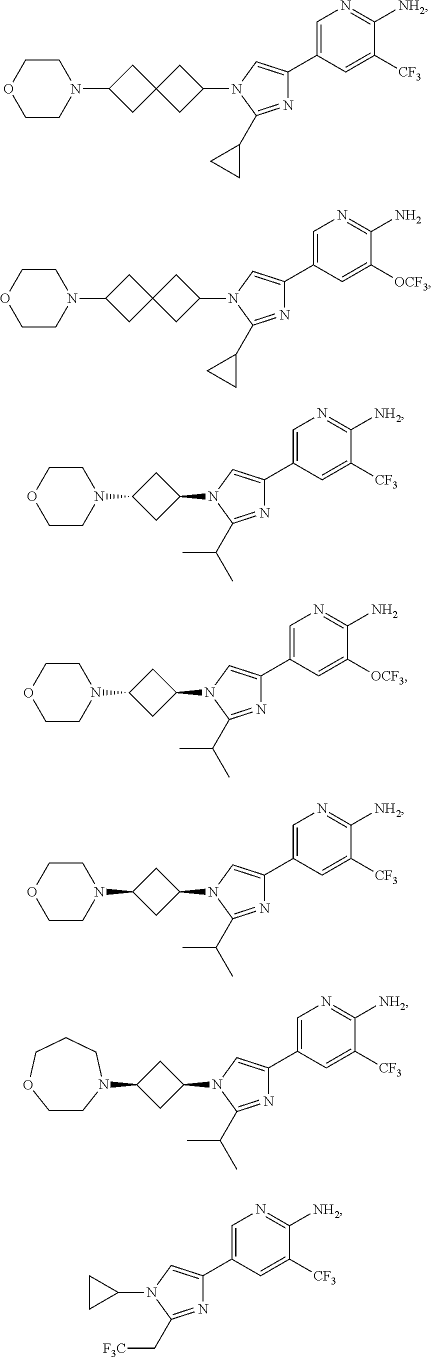 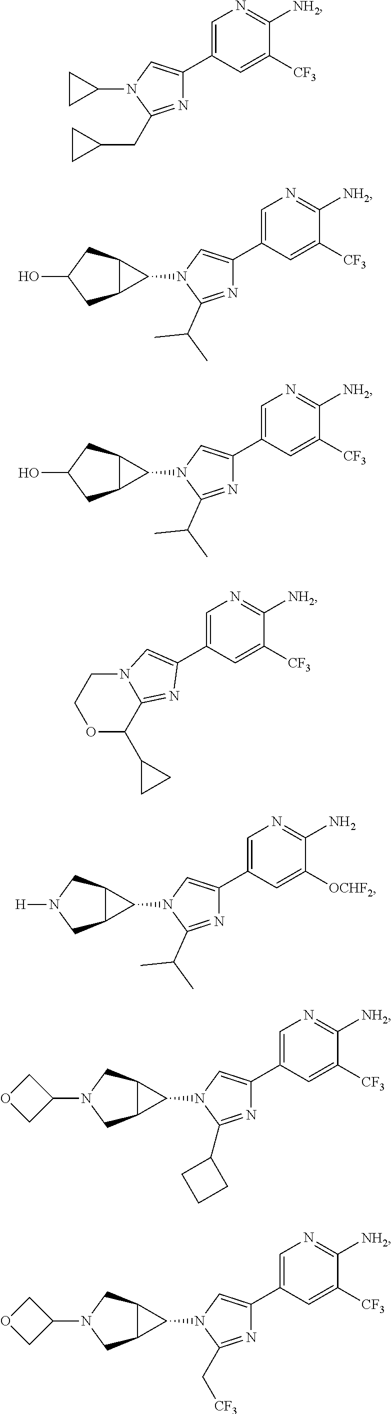 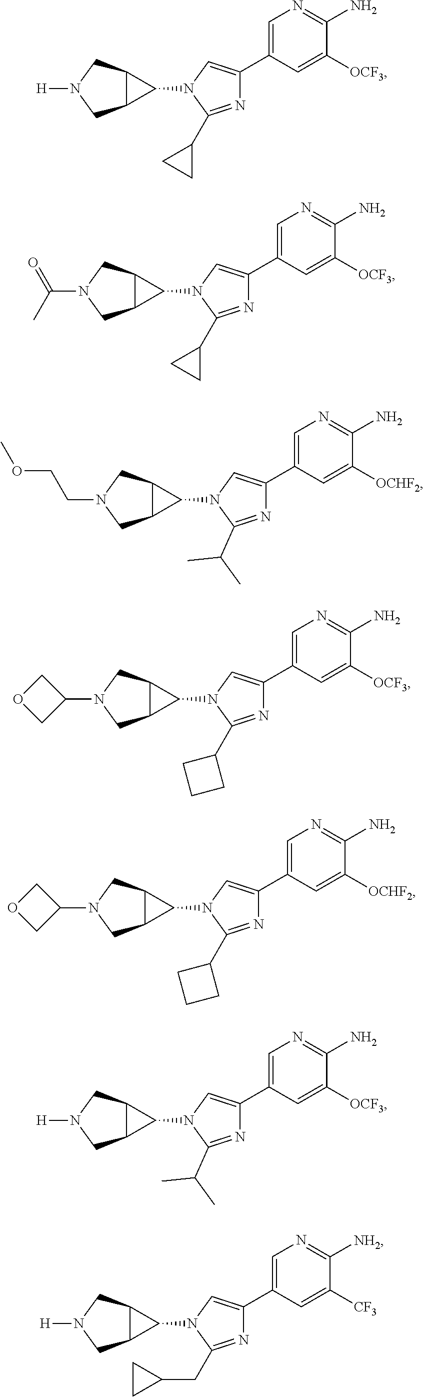 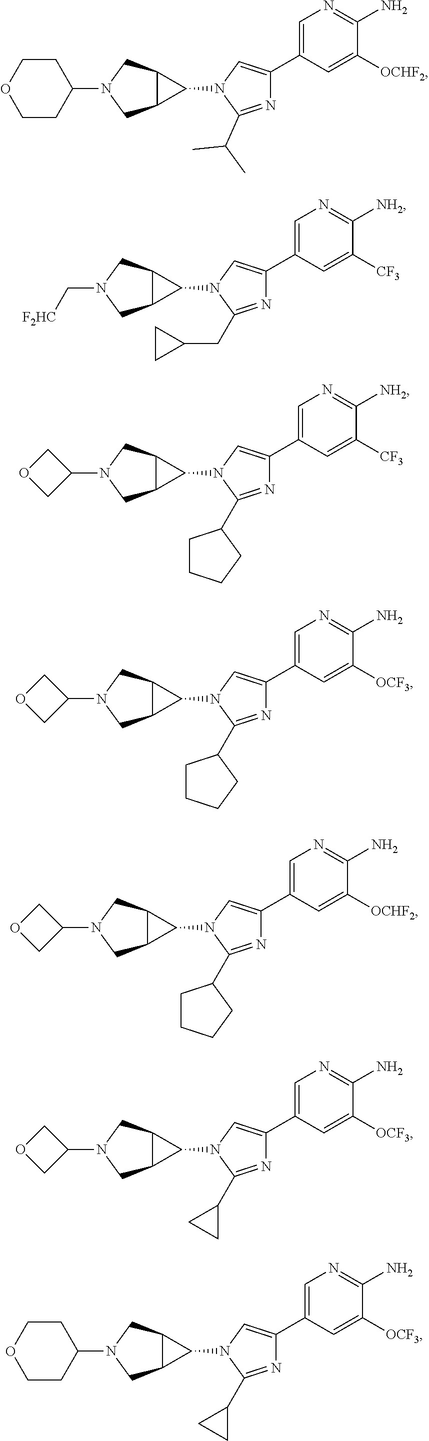 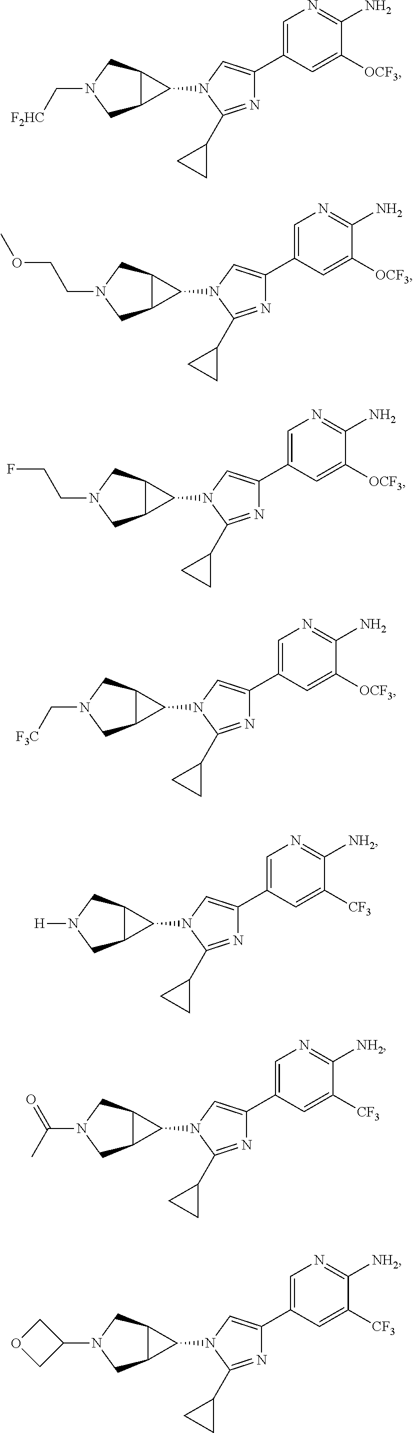 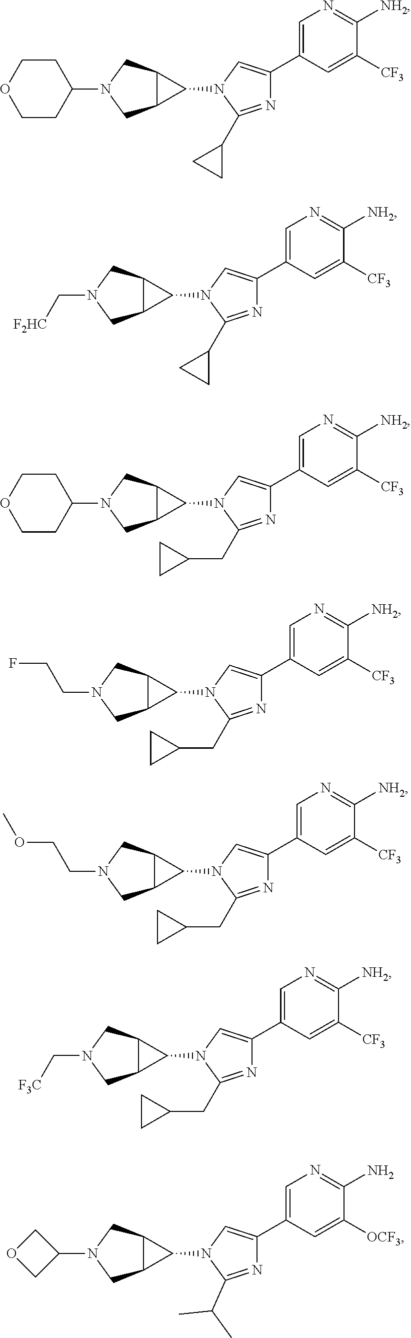 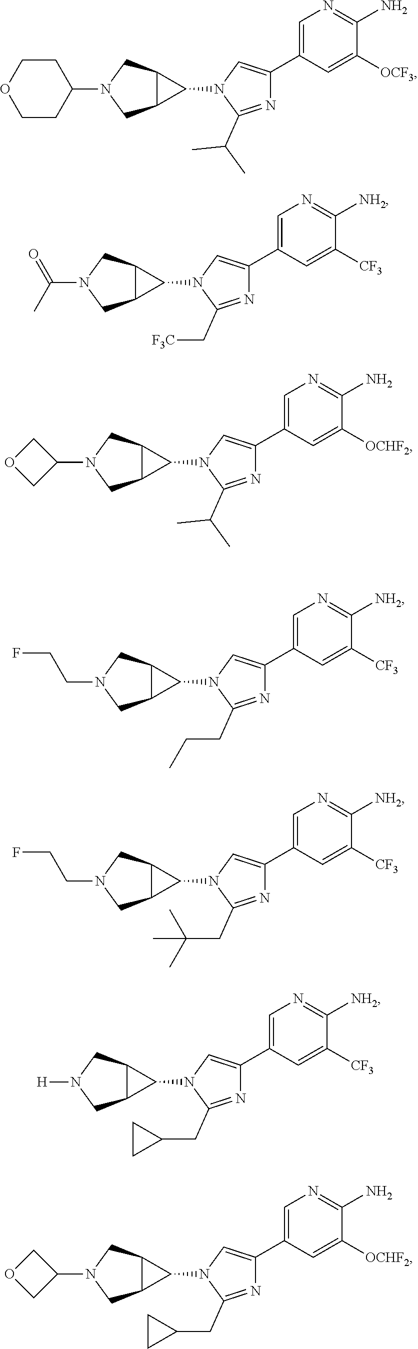 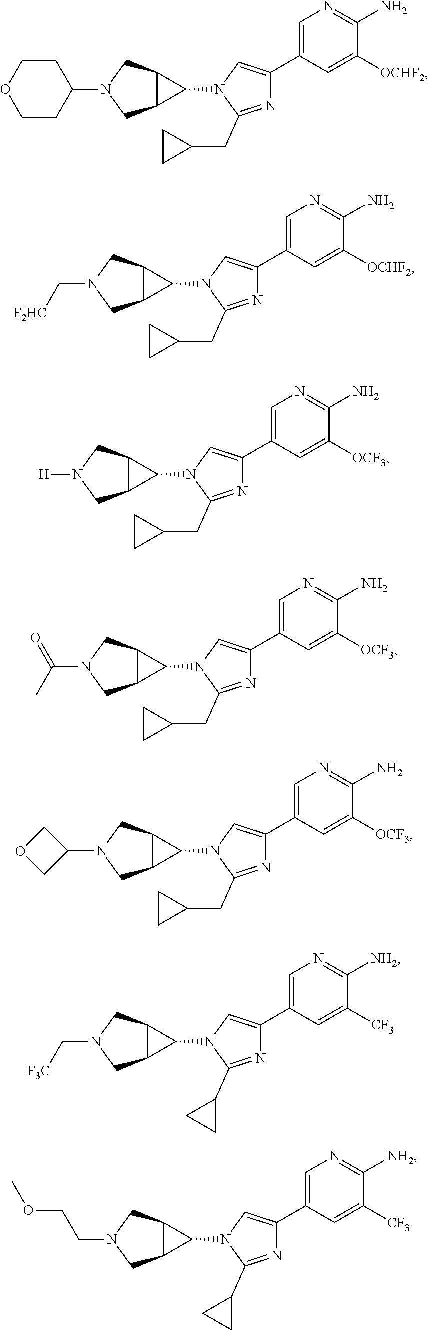 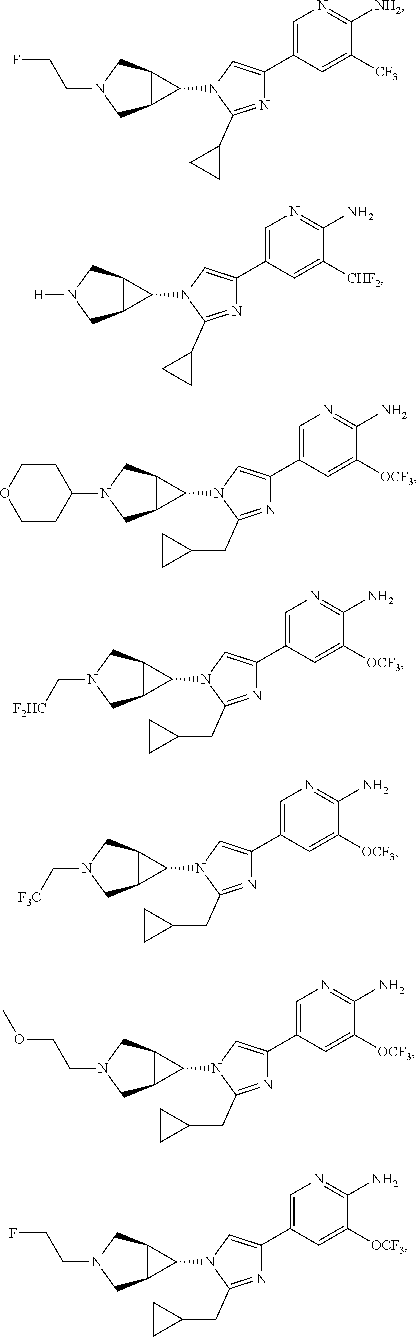 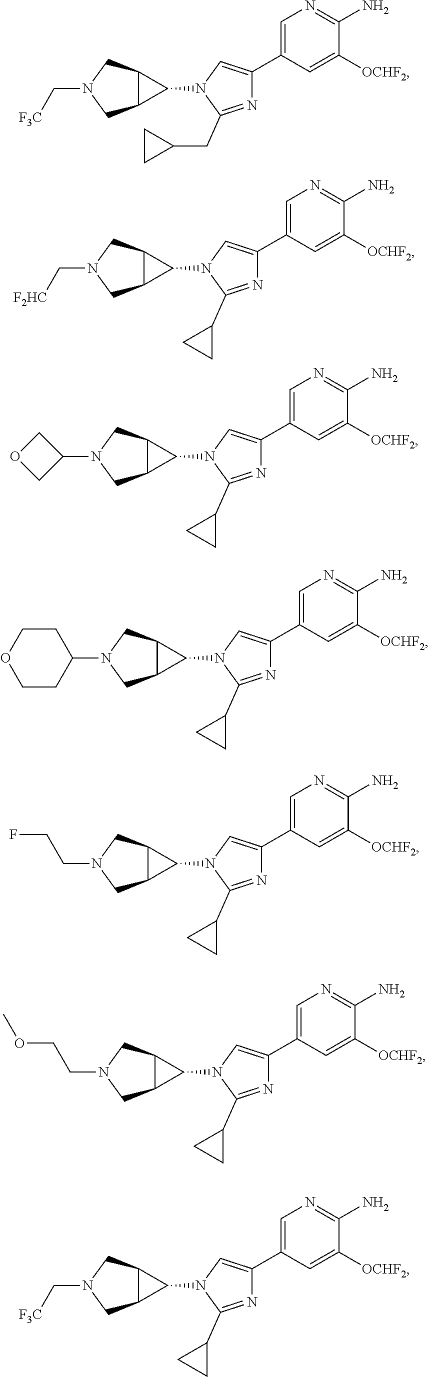 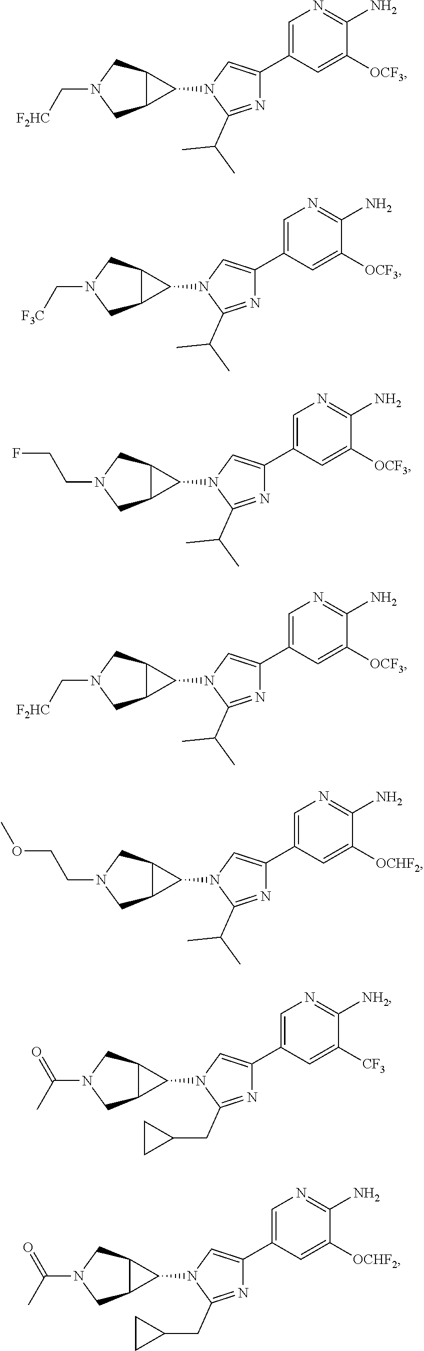 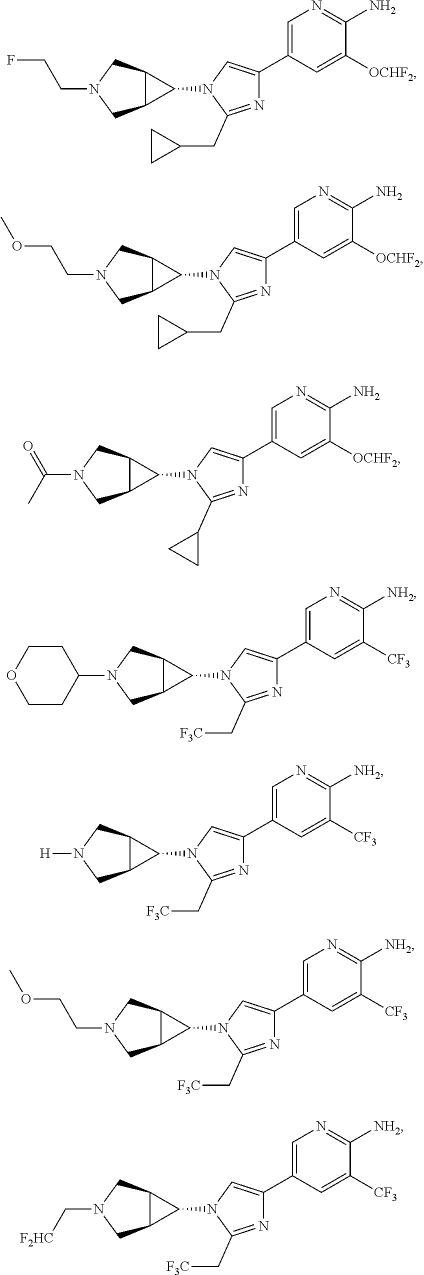 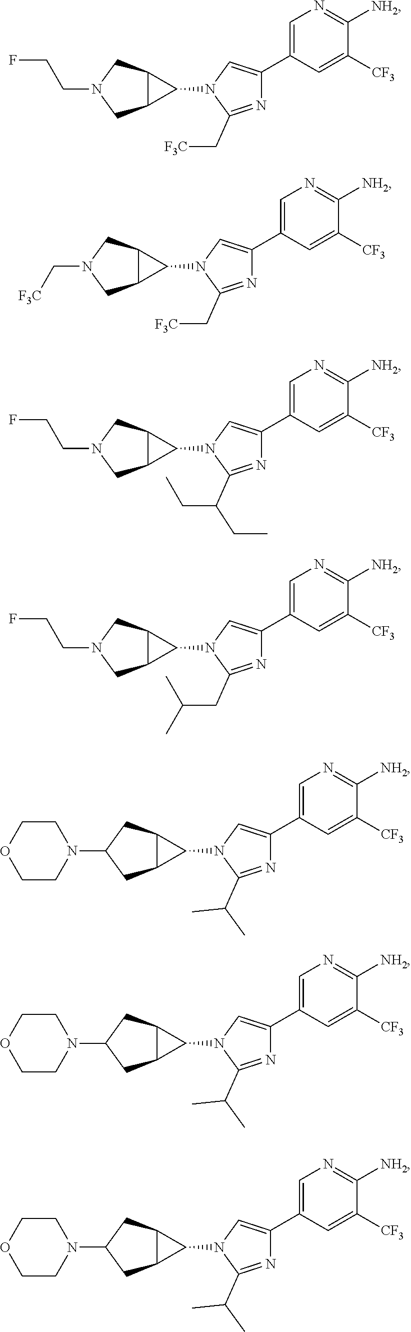 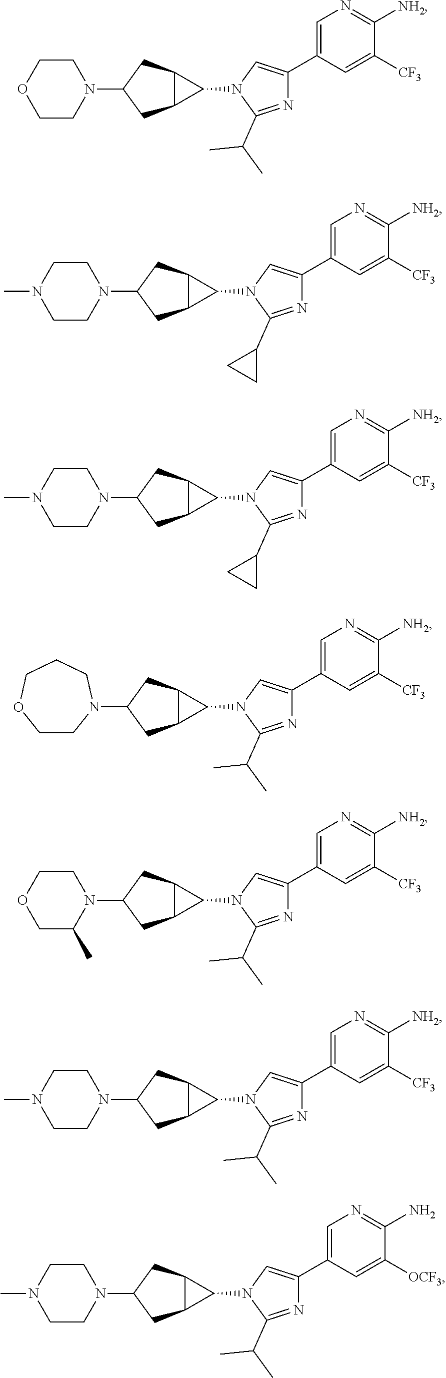 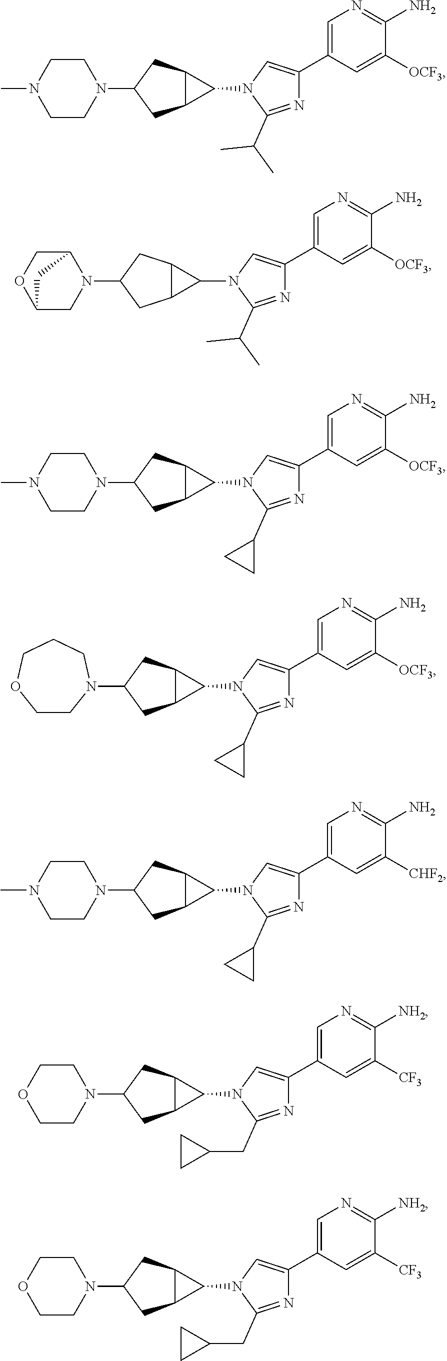 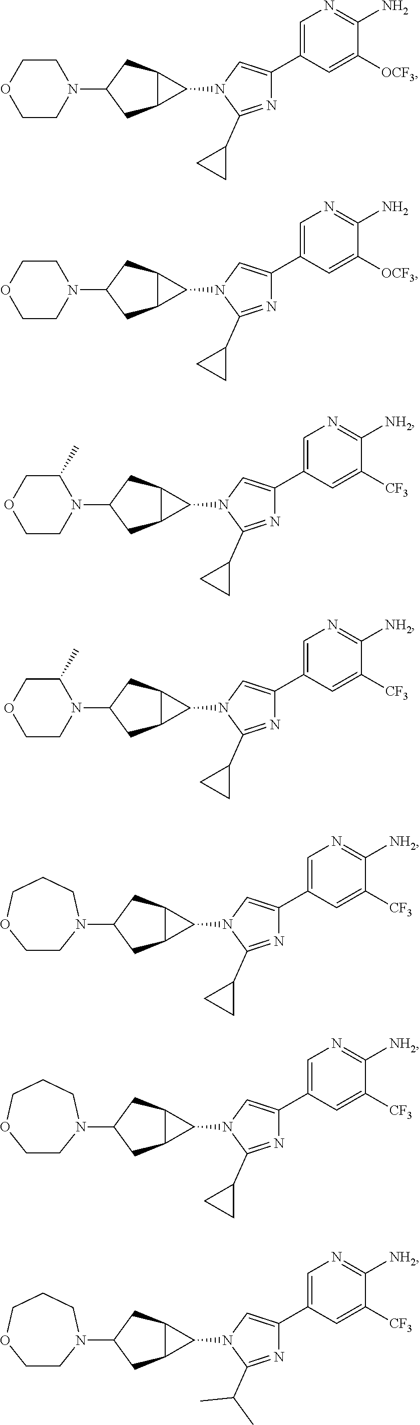 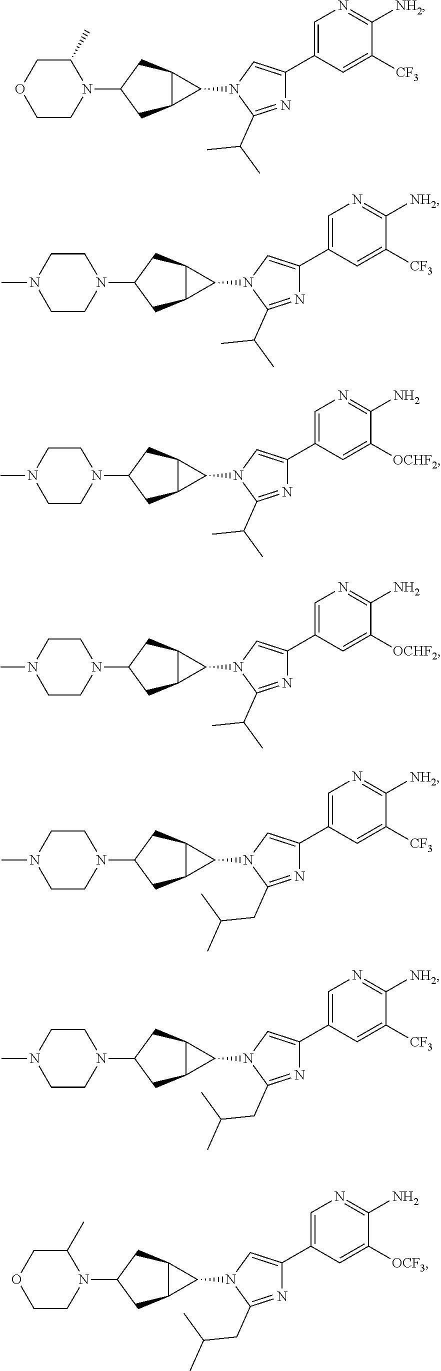 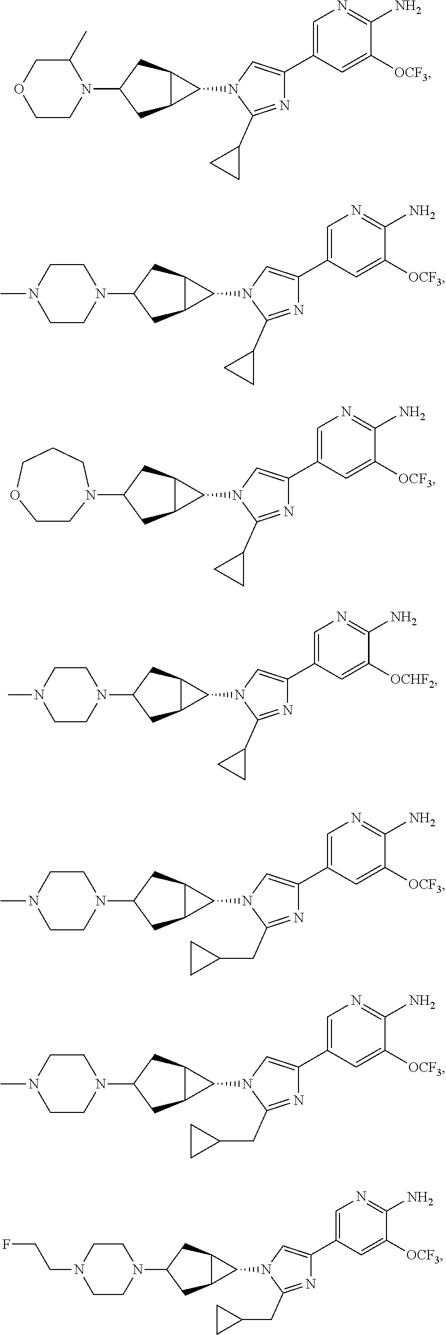 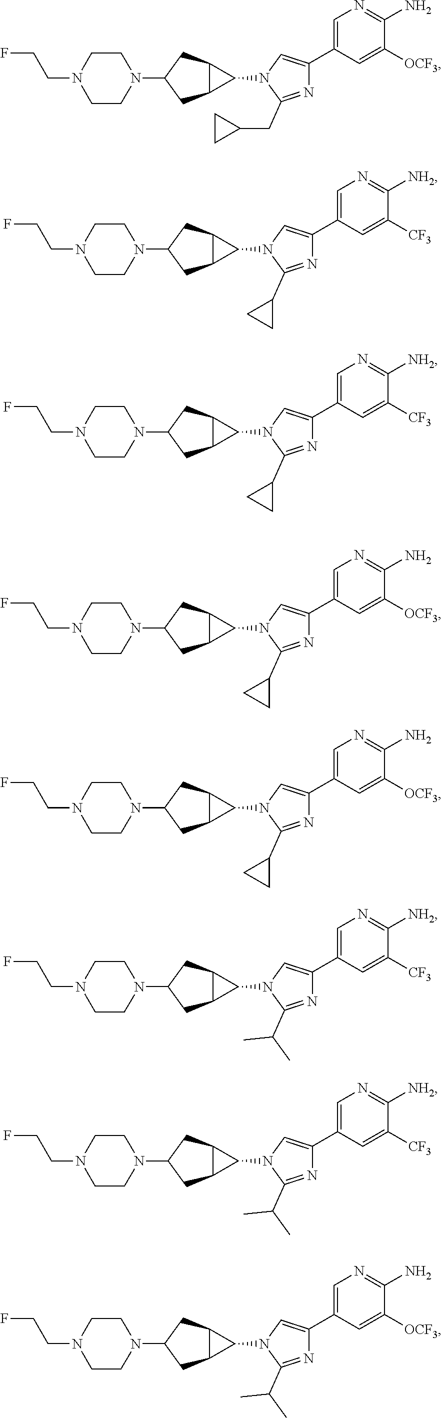 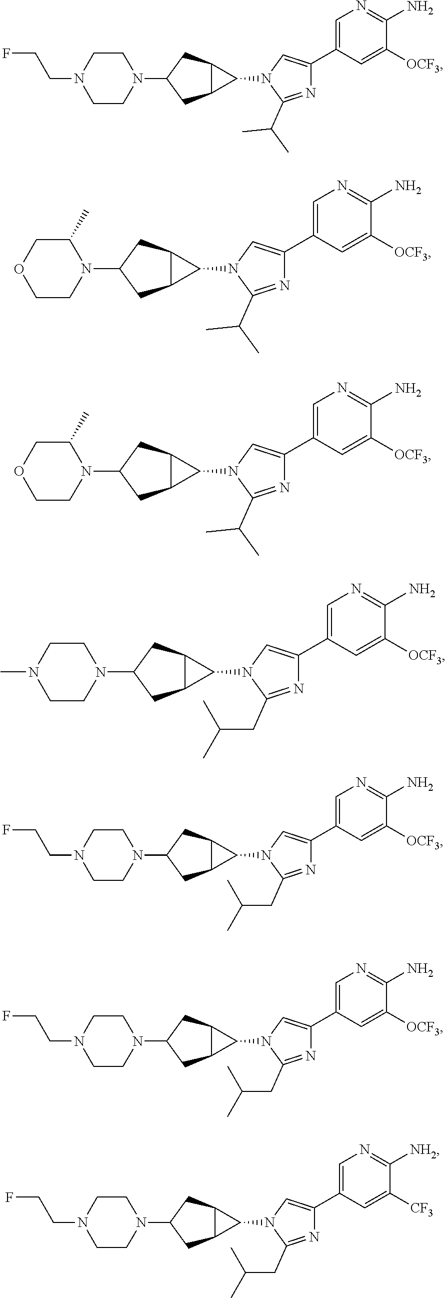 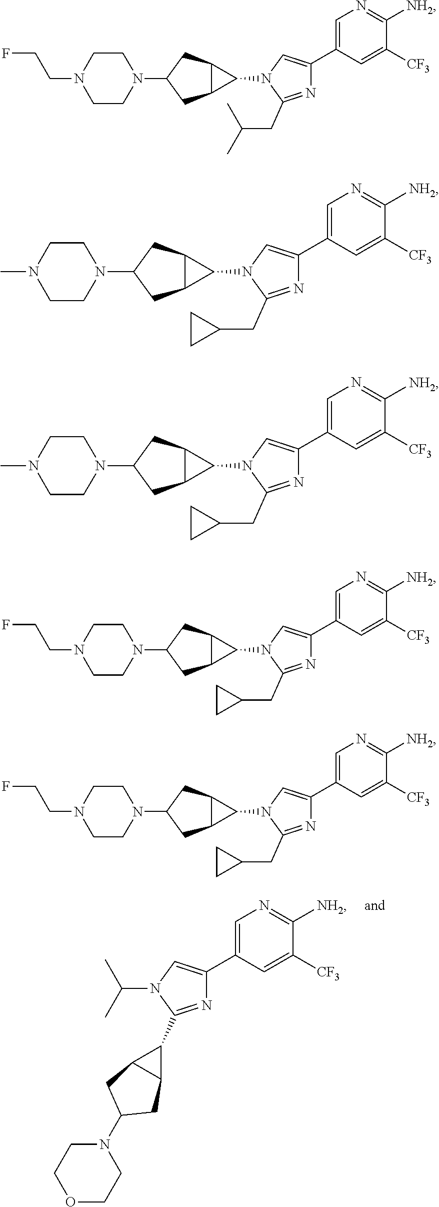 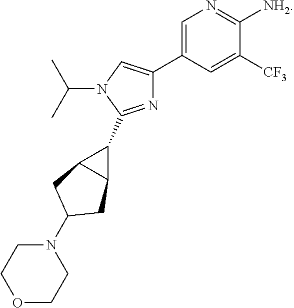
or a salt or ester thereof.

27. The compound of claim 1, wherein the compound has the structural formula chosen from: 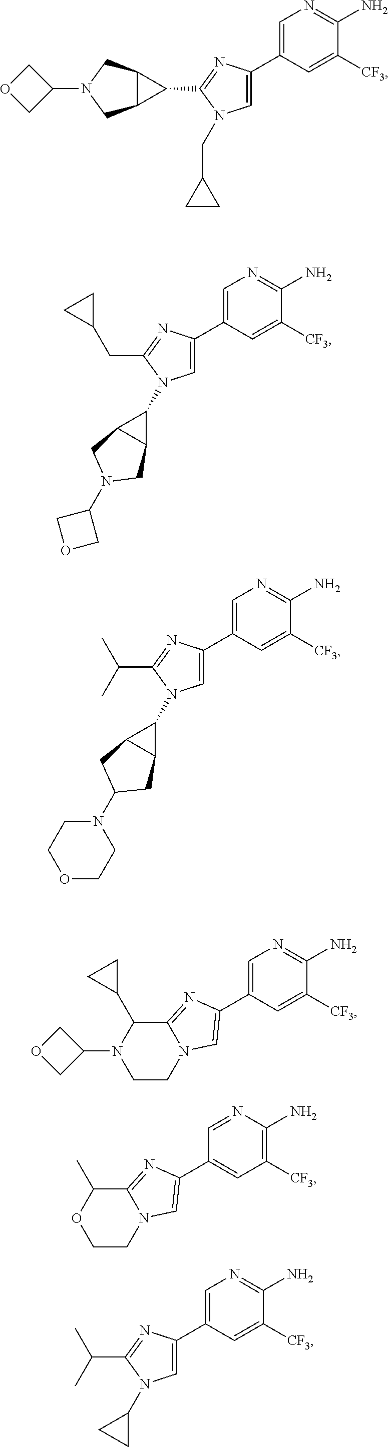 [SMILES: Nc1ncc(-c2cn(CC3CC3)c([C@H]3[C@@H]4CN(C5COC5)C[C@@H]43)n2)cc1C(F)(F)F ; Nc1ncc(-c2cn([C@H]3[C@@H]4CN(C5COC5)C[C@@H]43)c(CC3CC3)n2)cc1C(F)(F)F ; CC(C)c1nc(-c2cnc(N)c(C(F)(F)F)c2)cn1[C@H]1[C@@H]2CC(N3CCOCC3)C[C@@H]21 ; Nc1ncc(-c2cn3c(n2)C(C2CC2)N(C2COC2)CC3)cc1C(F)(F)F ; CC1OCCn2cc(-c3cnc(N)c(C(F)(F)F)c3)nc21 ; CC(C)c1nc(-c2cnc(N)c(C(F)(F)F)c2)cn1C1CC1] 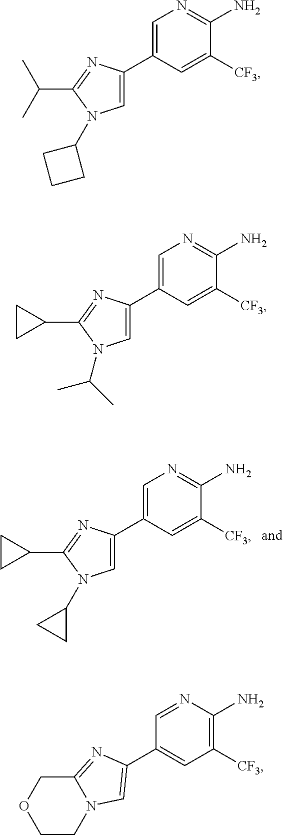 [SMILES: CC(C)c1nc(-c2cnc(N)c(C(F)(F)F)c2)cn1C1CCC1 ; CC(C)n1cc(-c2cnc(N)c(C(F)(F)F)c2)nc1C1CC1 ; Nc1ncc(-c2cn(C3CC3)c(C3CC3)n2)cc1C(F)(F)F ; Nc1ncc(-c2cn3c(n2)COCC3)cc1C(F)(F)F]
or a salt or ester thereof.

28. A compound as recited in claim 1 for use as a medicament.

29. A compound as recited in claim 1 for use in the manufacture of a medicament for the prevention or treatment of a disease or condition ameliorated by the inhibition of DLK.

30. A compound as recited in claim 1 for use in the treatment of a disease mediated by DLK kinase.

31. The compound as recited in claim 30, wherein said disease results from traumatic injury to central nervous system or peripheral nervous system neurons.

32. The compound as recited in claim 31, wherein said traumatic injury is chosen from stroke, traumatic brain injury, and spinal cord injury.

33. The compound as recited in claim 30, wherein said disease results from a chronic neurodegenerative condition.

34. The compound as recited in claim 33, wherein said neurodegenerative condition is chosen from Alzheimer's disease, frontotemporal dementia, Parkinson's disease, Huntington's disease, amyotrophic lateral sclerosis, spinocerebellar ataxia, progressive supranuclear palsy, Lewy body disease, chemotherapy-induced peripheral neuropathy, diabetic neuropathy and Kennedy's disease.

35. The compound as recited in claim 30, wherein said disease results from a neuropathy resulting from neurological damage.

36. The compound as recited in claim 35, wherein said neurological damage is chosen from chemotherapy-induced peripheral neuropathy and diabetic neuropathy.

37. A compound as recited in claim 1 for the use in the treatment of a cognitive disorder.

38. The compound as recited in claim 37, wherein said cognitive disorder is caused by pharmacological intervention.

39. A pharmaceutical composition comprising a compound as recited in claim 1 together with a pharmaceutically acceptable carrier.

40. A method of inhibition of DLK comprising contacting DLK with a compound as recited in claim 1.

41. A method of treatment of a DLK-mediated disease comprising the administration of a therapeutically effective amount of a compound as recited in claim 1 to a patient in need thereof.

42. The method as recited in claim 41 wherein said disease is a neurological disease.

43. The method as recited in claim 42, wherein said neurological disease results from traumatic injury to central nervous system or peripheral nervous system neurons.

44. The method as recited in claim 43, wherein said traumatic injury is chosen from stroke, traumatic brain injury, and spinal cord injury.

45. The method as recited in claim 42, wherein said neurological disease results from a chronic neurodegenerative condition.

46. The method as recited in claim 45, wherein said chronic neurodegenerative condition is chosen from Alzheimer's disease, frontotemporal dementia, Parkinson's disease, Huntington's disease, amyotrophic lateral sclerosis, spinocerebellar ataxia, progressive supranuclear palsy, Lewy body disease, and Kennedy's disease.

47. The method as recited in claim 42, wherein said neurological disease results from a neuropathy resulting from neurological damage.

48. The method as recited in claim 47, wherein said neurological damage is chosen from chemotherapy-induced peripheral neuropathy and diabetic neuropathy.

49. The method as recited in claim 41 wherein said disease is a cognitive disorder.

50. The method as recited in claim 49 wherein said cognitive disorder is caused by pharmacological intervention

51. A method of treatment of a DLK-mediated disease comprising the administration of:
a. a therapeutically effective amount of a compound as recited in claim 1; and
b. another therapeutic agent.

52. The method as recited in claim 51, wherein said DLK-mediated disease is a cognitive disorder caused by pharmacological intervention.

53. The method as recited in claim 52, wherein said cognitive disorder is chemotherapy-induced cognitive disorder.

54. A method for achieving an effect in a patient comprising the administration of a therapeutically effective amount of a compound as recited in claim 1 to a patient, wherein the effect is chosen from decrease loss of neurons, reduction in cerebral atrophy, improved neurological function, improved cognition, and improved mental performance.
